SEQUENCE LISTING (28 sequences)

SEQ ID NO: 1 - protein, 182 aa, Homo sapiens
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPTQGASPSYAS

SEQ ID NO: 3 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPTQGAMPSYAS

SEQ ID NO: 4 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPTPPDVGSSDPLSMVGPSQGRSPSYAS

SEQ ID NO: 5 - protein, 182 aa, Artificial
  mutant
MHPIPTSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 6 - protein, 182 aa, Artificial
  mutant
MHPTPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 7 - protein, 182 aa, Artificial
  mutant
MHPIPDSSPTLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 8 - protein, 182 aa, Artificial
  mutant
MHPIPDSSPLLQFGGQVRQRYLYTDDAQQTEAHLEIREDGTVGGAADQSPTSLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 9 - protein, 182 aa, Artificial
  mutant
MHPIPDSSPLLQFGGQVRQRYLYTDDAQQTEAHLEIREDGTVGGAADQSPESLLQLKALKPTVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 10 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPTGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 11 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPTACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 12 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPTHLPGNKSPHRDPAPRGPARFLPLPGLPPALPEPPGILAPQPPDVGSSDPLSMVGPSQGRSPSYAS

SEQ ID NO: 13 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPTNKSPHRDPAPRGPARFLPLPGLPPALPEPPGILAPQPPDVGSSDPLSMVGPSQGRSPSYAS

SEQ ID NO: 14 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPTRDPAPRGPARFLPLPGLPPALPEPPGILAPQPPDVGSSDPLSMVGPSQGRSPSYAS

SEQ ID NO: 15 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPTPRGPARFLPLPGLPPALPEPPGILAPQPPDVGSSDPLSMVGPSQGRSPSYAS

SEQ ID NO: 16 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPTGPARFLPLPGLPPALPEPPGILAPQPPDVGSSDPLSMVGPSQGRSPSYAS

SEQ ID NO: 17 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPTRFLPLPGLPPALPEPPGILAPQPPDVGSSDPLSMVGPSQGRSPSYAS

SEQ ID NO: 18 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPTPGLPPALPEPPGILAPQPPDVGSSDPLSMVGPSQGRSPSYAS

SEQ ID NO: 19 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPTLPPALPEPPGILAPQPPDVGSSDPLSMVGPSQGRSPSYAS

SEQ ID NO: 20 - protein, 183 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPTPALPEPPGILAPQPPDVGSSDPLSMVGPSQGRSPSYAS

SEQ ID NO: 21 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPTLPEPPGILAPQPPDVGSSDPLSMVGPSQGRSPSYAS

SEQ ID NO: 22 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPTPPGILAPQPPDVGSSDPLSMVGPSQGRSPSYAS

SEQ ID NO: 23 - protein, 183 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPTPGILAPQPPDVGSSDPLSMVGPSQGRSPSYAS

SEQ ID NO: 24 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPTILAPQPPDVGSSDPLSMVGPSQGRSPSYAS

SEQ ID NO: 25 - protein, 183 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPTPDVGSSDPLSMVGPSQGRSPSYAS

SEQ ID NO: 26 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPTVGSSDPLSMVGPSQGRSPSYAS

SEQ ID NO: 27 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPTSMVGPSQGRSPSYAS

SEQ ID NO: 28 - protein, 182 aa, Artificial
  mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPTYAS

(19) United States
(12) Patent Application Publication
(10) Pub. No.: US 2023/0090114 A1
(43) Pub. Date: Mar. 23, 2023

(54) METHODS FOR PROMOTING WEIGHT LOSS

(71) Applicant: 89BIO LTD., Herzliya (IL)

(72) Inventors: Mordechay ROSENSTOCK, Talmei Elazar (IL); Maya MARGALIT, Tel-Aviv (IL)

(21) Appl. No.: 17/793,953

(22) PCT Filed: Jan. 29, 2021
(86) PCT No.: PCT/IB2021/000044

§ 371 Date: Jul. 20, 2022

Related U.S. Application Data
(60) Provisional application No. 62/968,594, filed on Jan. 31, 2020.

(51) Int. Cl. A61K 38/18; A61K 47/60; A61P 3/04

(52) U.S. Cl. CPC A61K 38/1825; A61K 47/60; A61P 3/04

(57) ABSTRACT

Therapeutic regimens and uses of mutant Fibroblast Growth Factor-21 (FGF-21) peptide conjugates comprising a polyethylene glycol (PEG) moiety attached to a mutant FGF-21 peptide via a glycosyl moiety thereof in the reduction of total body weight, reduction of body fat content, and/or reduction of BMI of the subject in need thereof are provided.

### RELATED APPLICATIONS

This application claims the benefit of and priority to U.S. provisional Application Ser. No. 62/968,594, filed Jan. 31, 2020, the disclosure of which is incorporated herein by reference in its entirety.

### SEQUENCE LISTING

The instant application contains a Sequence Listing, submitted herewith which includes the file 180234-011201PCT_ST25.txt having the following size 46,270 bytes, which was created on Jan. 27, 2021, the contents of which are hereby incorporated by reference herein.

### TECHNICAL FIELD

The present disclosure relates to therapeutic regimens and uses of mutant Fibroblast Growth Factor-21 (FGF-21) peptide conjugates comprising a polyethylene glycol (PEG) moiety attached to a mutant FGF-21 peptide via a glycosyl moiety thereof for promoting weight loss in a subject in need thereof. In a particular embodiment, the subject does not display characteristics associated with diabetes type 2, non-alcoholic steatohepatitis (NASH), and/or metabolic syndrome. Characteristics associated with diabetes type 2, non-alcoholic steatohepatitis (NASH), and/or metabolic syndrome are known in the art and are described herein.

### BACKGROUND

Maintaining a desired weight is challenging for many people. Moreover, as people age, maintaining a desired weight typically becomes increasingly more difficult. Excess or unwanted body weight in a person can result from an imbalance in caloric (energy) intake and energy expenditure. Such imbalances may be associated with a variety of factors, including: overeating, an inactive or sedentary lifestyle, familial genetics, and various medical conditions. Further compounding the challenge of maintaining a desired weight and desired physique, basal metabolic rate (BMR) typically declines after age 20, mostly due to loss of fat free mass.

Reduction of total dietary caloric intake can be achieved via a diet regimen that includes careful monitoring and restriction of calories consumed. Implementation of an exercise program may also assist in maintaining a desired weight. Such an approach may be combined with administration of drugs or supplements that act as, for example, caloric blockers, meal replacements, and/or appetite suppressants. The effectiveness of currently available drugs and supplements for promoting weight control or weight loss is, however, unpredictable. Efficacy of such drugs and supplements is particularly problematic if they are not used in conjunction with a calorie-restricted diet and exercise regimen.

The dearth of effective drugs and supplements for promoting weight control or weight loss, therefore, contributes to the challenges of body weight management for many people.

### SUMMARY

In some embodiments, provided herein are methods comprising administering once a week to a subject in need thereof a pharmaceutical composition comprising from 0.08 mg/kg to 1 mg/kg of a mutant Fibroblast Growth Factor-21 (FGF-21) peptide conjugate and a pharmaceutically acceptable carrier, wherein the subject is in need of reduction of total body weight, reduction of body fat content, reduction of body mass index (BMI), or combination thereof; wherein the mutant FGF-21 peptide conjugate comprises: i) a mutant FGF-21 peptide comprising the amino acid sequence of SEQ ID NO: 2, ii) a glycosyl moiety, and iii) a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG, wherein administration of the pharmaceutical composition results in at least one of: reduction of total body weight, reduction of body fat content, reduction of BMI of the subject or combination thereof.

In some embodiments, provided herein are methods comprising administering once every two weeks to a subject in need thereof a pharmaceutical composition comprising from 0.08 mg/kg to 1 mg/kg of a mutant Fibroblast Growth Factor-21 (FGF-21) peptide conjugate and a pharmaceutically acceptable carrier, wherein the subject is in need of reduction of total body weight, reduction of body fat content, reduction of body mass index (BMI), or combination thereof, wherein the mutant FGF-21 peptide conjugate comprises: i) a mutant FGF-21 peptide comprising the amino acid sequence of SEQ ID NO: 2, ii) a glycosyl moiety, and iii) a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG, wherein administration of the pharmaceutical composition results in at least one of: reduction of total body weight, reduction of body fat content, reduction of BMI of the subject or combination thereof.

In some embodiments, administration of the pharmaceutical composition increases of thermogenesis, decreases in fat mass without affecting lean masses, decreases in fat mass without affecting body fluid or combinations thereof.

In some embodiments, the subject is a human subject. In some embodiments, the subject is not afflicted with diabetes, NASH, and/or metabolic syndrome. In some embodiments, the subject has a BMI ranging from 25 to less than 30. In some embodiments, the subject has a BMI of less than 25. In some embodiments, the subject has an HbA1C level within normal range of from 4% to 5.6%. In some embodiments, the subject has a BMI of 30 or greater, and does not have diabetes, NASH, or metabolic syndrome.

In some embodiments, the mutant FGF-21 peptide conjugate exhibit a half life of about 80 hours or greater.

In some embodiments, the pharmaceutical composition is administered in combination with a weight loss therapeutic agent.

In some embodiments, the pharmaceutical composition is administered sub-subcutaneously.

In some embodiments, the glycosyl moiety comprises at least one of an N-acetylgalactosamine (GalNAc) residue, a galactose (Gal) residue, a sialic acid (Sia) residue, a 5-amine analogue of a Sia residue, a mannose (Man) residue, mannosamine, a glucose (Glc) residue, an N-acetylglucosamine (GlcNAc) residue, a fucose residue, a xylose residue, or a combination thereof. In some embodiments, the glycosyl moiety comprises at least one of an N-acetylgalactosamine (GalNAc) residue, a galactose (Gal) residue, a sialic acid (Sia), or a combination thereof. In some embodiments, the at least one Sia residue is a nine-carbon carboxylated sugar. In some embodiments, the at least one Sia residue is N-acetyl-neuraminic acid (2-keto-5-acetamido-3,5-dideoxy-D-glycero-D-galactononulopyranos-1-onic acid (Neu5Ac), N-glycolylneuraminic acid (Neu5Gc), 2-keto-3-deoxy-nonulosonic acid (KDN), or a 9-substituted sialic acid. In some embodiments, the 9-substituted sialic acid is 9-O-lactyl-Neu5Ac, 9-O-acetyl-Neu5Ac, 9-deoxy-9-fluoro-Neu5Ac, or 9-azido-9-deoxy-Neu5Ac. In some embodiments, the glycosyl moiety comprises the structure -GalNAc-Sia-. In some embodiments, the 20 kDa PEG moiety is attached to the glycosyl moiety by a covalent bond to a linker, wherein the linker comprises at least one amino acid residue. In some embodiments, the at least one amino acid residue is a glycine (Gly).

In some embodiments, the mutant FGF-21 comprises the structure -GalNAc-Sia-Gly-PEG (20 kDa).

In some embodiments, the mutant FGF-21 comprises the structure:

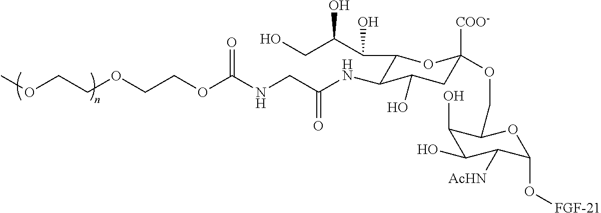

wherein n is an integer selected from 450 to 460.

In some embodiments, the 20 kDa PEG is a linear or branched PEG. In some embodiments, the 20 kDa PEG is a 20 kDa methoxy-PEG.

### BRIEF DESCRIPTION OF THE DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIGS. 1A-1B are graphs showing the impact of 89BIO-100 on the body weight (g) (FIG. 1A) and delta body weight (g) (FIG. 1i). (A) of the Vehicle (n=7; grey curves), Liraglutide (n=8; purple curves), 89BIO-100 0.3 mg/kg (n=11; light blue curves), 89BIO-100 1 mg/kg (n=11; blue curves) and 89BIO-100 3 mg/kg (n=8; dark blue curves). Data are represented as Mean±SEM. Orange squares (T1, T3, T5, T8, T10, T12, T14, T17, T19, T22, T24m T26) represent the days of 89BIO-100 treatment.

FIGS. 2A-2B are graphs showing the impact of 89BIO-100 on the cumulative food intake (g) during T₁. One hour-resolution recordings of (A) cumulative food intake (g) (FIG. 2A) and (B) histograms of mean food intake (FIG. 2B), adapted from the high-resolution recordings shown in FIG. 2A of the Vehicle (n=6; grey curves/bars), Liraglutide (n=8; purple curves/bars), 89BIO-100 0.3 mg/kg (n=8; light blue curves/bars), 89BIO-100 1 mg/kg (n=8; blue curves/bars) and 89BIO-100 3 mg/kg (n=8; dark blue curves/bars) during the diurnal phase (DP), the nocturnal phase (NP) and the whole day (WD) on T₁. Data are represented as Mean±SEM. * p<0.05, significantly different from the Vehicle group; ^{##} p<0.01; ^{###} p<0.001, significantly different from the Liraglutide group. Purple rectangle represents the nocturnal phase.

FIGS. 3A-3F show meal pattern analysis during T₁. (FIG. 3A) Mean meal number, (FIG. 3B) mean meal size (g), (FIG. 3C) mean meal duration (min), (FIG. 3D) mean post-meal interval (min), (FIG. 3E) mean eating rate (g/min) and (FIG. 3F) satiety ratio (min/g) of the Vehicle (n=6; grey bars), Liraglutide (n=8; purple bars), 89BIO-100 0.3 mg/kg (n=8; light blue bars), 89BIO-100 1 mg/kg (n=8; blue bars) and 89BIO-100 3 mg/kg (n=8; dark blue bars) during the whole day (WD), the diurnal phase (DP) and the nocturnal phase (NP) on T₁. Data are represented as Mean±SEM. *p<0.05, significantly different from the Vehicle group; ^{#} p<0.05; ^{##} p<0.01, significantly different from the Liraglutide group, $$ p<0.01, significantly different from the 89BIO-100 0.3 mg/kg group, ° p<0.05, significantly different from the 89BIO-100 1 mg/kg group.

FIGS. 4A-4B show the impact of 89BIO-100 on the cumulative food intake (g) during T₂. One hour-resolution recordings of (FIG. 4A) cumulative food intake (g) and (FIG. 4B) histograms of mean food intake, adapted from the high-resolution recordings shown in FIG. 4A of the Vehicle (n=8; grey curves/bars), Liraglutide (n=8; purple curves/bars), 89BIO-100 0.3 mg/kg (n=11; light blue curves/bars), 89BIO-100 1 mg/kg (n=11; blue curves/bars) and 89BIO-100 3 mg/kg (n=8; dark blue curves/bars) during the diurnal phase (DP), the nocturnal phase (NP) and the whole day (WD) on T₂. Data are represented as Mean±SEM. ** p<0.01; *** p<0.001 significantly different from the Vehicle group; ^{##} p<0.01; ^{###} p<0.001, significantly different from the Liraglutide group. Purple rectangle represents the nocturnal phase.

FIGS. 5A-5F show meal pattern analysis during T₂. (FIG. 5A) Mean meal number, (FIG. 5B) mean meal size (g), (FIG. 5C) mean meal duration (min), (FIG. 5D) mean post-meal interval (min), (FIG. 5E) mean eating rate (g/min) and (FIG. 5F) satiety ratio (min/g) of the Vehicle (n=8; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars) during the whole day (WD), the diurnal phase (DP) and the nocturnal phase (NP) on T₂. Data are represented as Mean±SEM. *p<0.05; **p<0.01, significantly different from the Vehicle group; ^{#} p<0.05; ^{##} p<0.01; ^{###} p<0.001, significantly different from the Liraglutide group.

FIGS. 6A-6F show the impact of 89BIO-100 on the meal pattern analysis for the first meal during T₂₆. (FIG. 6A) First meal latency (min), (FIG. 6B) mean meal size (g), (FIG. 6C) mean meal duration (min), (FIG. 6D) mean post-meal interval (min), (FIG. 6E) mean eating rate (g/min) and (FIG. 6F) satiety ratio (min/g) of the Vehicle (n=7; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars) on T₂₆. Data are represented as Mean±SEM. *p<0.05; **p<0.01, significantly different from the Vehicle group; ^{#} p<0.05, significantly different from the Liraglutide group.

FIGS. 7A-7F show the impact of 89BIO-100 on the meal pattern during T₂₇. (FIG. 7A) Mean meal number, (FIG. 7B) mean meal size (g), (FIG. 7C) mean meal duration (min), (D) mean post-meal interval (min), (FIG. 7E) mean eating rate (g/min) and (FIG. 7F) satiety ratio (min/g) of the Vehicle (n=7; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars) during the whole day (WD), the diurnal phase (DP) and the nocturnal phase (NP) on T₂₇. Data are represented as Mean±SEM. **p<0.01, significantly different from the Vehicle group; ^{$$} p<0.01, significantly different from the 89BIO-100 0.3 mg/kg group, [symbol] p<0.001, significantly different from the 89BIO-100 1 mg/kg group.

FIGS. 8A-8B show the impact of 89BIO-100 on the cumulative water intake (ml) during T₂₆. One hour-resolution recordings of (FIG. 8A) cumulative food intake (g) and (FIG. 8B) histograms of mean food intake, adapted from the high-resolution recordings shown in FIG. 8A of the Vehicle (n=7; grey curves/bars), Liraglutide (n=8; purple curves/bars), 89BIO-100 0.3 mg/kg (n=11; light blue curves/bars), 89BIO-100 1 mg/kg (n=11; blue curves/bars) and 89BIO-100 3 mg/kg (n=8; dark blue curves/bars) during the diurnal phase (DP), the nocturnal phase (NP) and the whole day (WD) on T₂₆. Data are represented as Mean±SEM. *p<0.05, significantly different from the Vehicle group; ^{##} p<0.01, significantly different from the Liraglutide group, ^{$}p<0.05, significantly different from the 89BIO-100 0.3 mg/kg group. Purple rectangle represents the nocturnal phase.

FIGS. 9A-9B show the impact of 89BIO-100 on the cumulative water intake (ml) during T₂₇. One hour-resolution recordings of (FIG. 9A) cumulative food intake (g) and (FIG. 9B) histograms of mean food intake, adapted from the high-resolution recordings shown in FIG. 9A of the Vehicle (n=7; grey curves/bars), Liraglutide (n=8; purple curves/bars), 89BIO-100 0.3 mg/kg (n=11; light blue curves/bars), 89BIO-100 1 mg/kg (n=11; blue curves/bars) and 89BIO-100 3 mg/kg (n=8; dark blue curves/bars) during the diurnal phase (DP), the nocturnal phase (NP) and the whole day (WD) on T₂₇. Data are represented as Mean±SEM. *p<0.05; **p<0.01, significantly different from the Vehicle group; ^{#} p<0.05; ^{##} p<0.01, significantly different from the Liraglutide group. Purple rectangle represents the nocturnal phase.

FIGS. 10A-10B show the impact of 89BIO-100 on the Energy expenditure (EE) and Respiratory exchange ratio (RER) during T₁. (FIG. 10A) Energy expenditure (EE) and (FIG. 10B) Respiratory exchange ratio (RER) profiles of the Vehicle (n=6; grey curves), Liraglutide (n=8; purple curves), 89BIO-100 0.3 mg/kg (n=11; light blue curves), 89BIO-100 1 mg/kg (n=11; blue curves) and 89BIO-100 3 mg/kg (n=8; dark blue curves) during T₁. Data are represented as Mean±SEM. Purple rectangles represent the nocturnal phase.

FIGS. 11A-11D show the impact of 89BIO-100 on the mean respiratory exchanges and related parameters during T₁. (FIG. 11A) Histograms of mean VO₂ were adapted from the high-resolution recordings, (FIG. 11B) histograms of mean VCO₂, adapted from the high-resolution recordings, (FIG. 11C) histograms of mean EE, adapted from the profiles shown in FIG. 10A, (FI. 11D) histograms of mean RER, adapted from the profiles shown in FIG. 10B of the Vehicle (n=6; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars) during T₁. Data are represented as Mean±SEM. *p<0.05; **p<0.01; ***p<0.001 significantly different from the Vehicle group; ^{#} p<0.05; ^{##} p<0.01; ^{###} p<0.001, significantly different from the Liraglutide group.

FIGS. 12A-12B show the impact of 89BIO-100 on the oxygen consumption (VO₂) and carbon dioxide production (VCO₂) during T₂. High-resolution recordings of (FIG. 12A) oxygen consumption (VO₂) and (FIG. 12B) carbon dioxide production (VCO₂) of the Vehicle (n=8; grey curves), Liraglutide (n=8; purple curves), 89BIO-100 0.3 mg/kg (n=11; light blue curves), 89BIO-100 1 mg/kg (n=11; blue curves) and 89BIO-100 3 mg/kg (n=8; dark blue curves) during T₂. Data are represented as Mean±SEM. Purple rectangles represent the nocturnal phase.

FIGS. 13A-13B show the impact of 89BIO-100 on the Energy expenditure (EE) and Respiratory exchange ratio (RER) during T₂. (FIG. 13A) Energy expenditure (EE) and (FIG. 13B) Respiratory exchange ratio (RER) profiles of the Vehicle (n=8; grey curves), Liraglutide (n=8; purple curves), 89BIO-100 0.3 mg/kg (n=11; light blue curves), 89BIO-100 1 mg/kg (n=11; blue curves) and 89BIO-100 3 mg/kg (n=8; dark blue curves) during T₂. Data are represented as Mean±SEM. Purple rectangles represent the nocturnal phase.

FIGS. 14A-14D show the impact of 89BIO-100 on the mean respiratory exchanges and related parameters during T₂. (FIG. 14A) Histograms of mean VO₂, adapted from the high-resolution recordings shown in FIG. 12A, (FIG. 14B) histograms of mean VCO₂, adapted from the high-resolution recordings shown in FIG. 12B, (FIG. 14C) histograms of mean EE, adapted from the profiles shown in FIG. 13A, (FIG. 14D) histograms of mean RER, adapted from the profiles shown in FIG. 13B of the Vehicle (n=8; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars) during T₂. Data are represented as Mean±SEM. ^{#} p<0.05; ^{##} p<0.01, significantly different from the Liraglutide group.

FIGS. 15A-15B show the impact of 89BIO-100 on the oxygen consumption (VO₂) and carbon dioxide production (VCO₂) during T₁₄. High-resolution recordings of (FIG. 15A) oxygen consumption (VO₂) and (FIG. 15B) carbon dioxide production (VCO₂) of the Vehicle (n=7; grey curves), Liraglutide (n=8; purple curves), 89BIO-100 0.3 mg/kg (n=11; light blue curves), 89BIO-100 1 mg/kg (n=11; blue curves) and 89BIO-100 3 mg/kg (n=8; dark blue curves) during T₁₄. Data are represented as Mean±SEM. Purple rectangles represent the nocturnal phase.

FIGS. 16A-16B show the impact of 89BIO-100 on the Energy expenditure (EE) and Respiratory exchange ratio (RER) during T₁₄. (FIG. 16A) Energy expenditure (EE) and (FIG. 16B) Respiratory exchange ratio (RER) profiles of the Vehicle (n=7; grey curves), Liraglutide (n=8; purple curves), 89BIO-100 0.3 mg/kg (n=11; light blue curves), 89BIO-100 1 mg/kg (n=11; blue curves) and 89BIO-100 3 mg/kg (n=8; dark blue curves) during T₁₄. Data are represented as Mean±SEM. Purple rectangles represent the nocturnal phase.

FIGS. 17A-17D show the impact of 89BIO-100 on the mean respiratory exchanges and related parameters during T₁₄. (FIG. 17A) Histograms of mean VO₂, adapted from the high-resolution recordings shown in FIG. 15A, (FIG. 17B) histograms of mean VCO₂, adapted from the high-resolution recordings shown in FIG. 15B, (FIG. 17C) histograms of mean EE, adapted from the profiles shown in FIG. 16A, (FIG. 17D) histograms of mean RER, adapted from the profiles shown in FIG. 16B of the Vehicle (n=7; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars) during T₁₄. Data are represented as Mean±SEM. * p<0.05; ** p<0.01, significantly different from the Vehicle group; ^{#} p<0.05; ^{##} p<0.01; ^{###} p<0.001, significantly different from the Liraglutide group.

FIGS. 18A-18B show the impact of 89BIO-100 on the oxygen consumption (VO₂) and carbon dioxide production (VCO₂) during T₁₅. High-resolution recordings of (FIG. 18A) oxygen consumption (VO₂) and (FIG. 18B) carbon dioxide production (VCO₂) of the Vehicle (n=5; grey curves), Liraglutide (n=8; purple curves), 89BIO-100 0.3 mg/kg (n=8; light blue curves), 89BIO-100 1 mg/kg (n=9; blue curves) and 89BIO-100 3 mg/kg (n=8; dark blue curves) during T₁₅. Data are represented as Mean±SEM. Purple rectangles represent the nocturnal phase.

FIGS. 19A-19B show the impact of 89BIO-100 on the Energy expenditure (EE) and Respiratory exchange ratio (RER) during T₁₅. (FIG. 19A) Energy expenditure (EE) and (FIG. 19B) Respiratory exchange ratio (RER) profiles of the Vehicle (n=5; grey curves), Liraglutide (n=8; purple curves), 89BIO-100 0.3 mg/kg (n=8; light blue curves), 89BIO-100 1 mg/kg (n=9; blue curves) and 89BIO-100 3 mg/kg (n=8; dark blue curves) during T₁₅. Data are represented as Mean±SEM. Purple rectangles represent the nocturnal phase.

FIGS. 20A-20D show the impact of 89BIO-100 on the mean respiratory exchanges and related parameters during T₁₅. (FIG. 20A) Histograms of mean VO₂, adapted from the high-resolution recordings shown in FIG. 18A, (FIG. 20B) histograms of mean VCO₂, adapted from the high-resolution recordings shown in FIG. 18B, (FIG. 20C) histograms of mean EE, adapted from the profiles shown in FIG. 19A, (FIG. 20D) histograms of mean RER, adapted from the profiles shown in FIG. 19B of the Vehicle (n=5; grey bars), Liraglutide (n=8; purple bars), 89BIO-100 0.3 mg/kg (n=8; light blue bars), 89BIO-100 1 mg/kg (n=9; blue bars) and 89BIO-100 3 mg/kg (n=8; dark blue bars) during T₁₅. Data are represented as Mean±SEM. * p<0.05; ** p<0.01, significantly different from the Vehicle group; ^{#} p<0.05; ^{##} p<0.01, significantly different from the Liraglutide group.

FIGS. 21A-21B show the impact of 89BIO-100 on the oxygen consumption (VO₂) and carbon dioxide production (VCO₂) during T₂₇. High-resolution recordings of (FIG. 21A) oxygen consumption (VO₂) and (FIG. 21B) carbon dioxide production (VCO₂) of the Vehicle (n=7; grey curves), Liraglutide (n=8; purple curves), 89BIO-100 0.3 mg/kg (n=11; light blue curves), 89BIO-100 1 mg/kg (n=11; blue curves) and 89BIO-100 3 mg/kg (n=8; dark blue curves) during T₂₇. Data are represented as Mean±SEM. Purple rectangles represent the nocturnal phase.

FIGS. 22A-22B show the impact of 89BIO-100 on the Energy expenditure (EE) and Respiratory exchange ratio (RER) during T₂₇. (FIG. 22A) Energy expenditure (EE) and (FIG. 22B) Respiratory exchange ratio (RER) profiles of the Vehicle (n=7; grey curves), Liraglutide (n=8; purple curves), 89BIO-100 0.3 mg/kg (n=11; light blue curves), 89BIO-100 1 mg/kg (n=11; blue curves) and 89BIO-100 3 mg/kg (n=8; dark blue curves) during T₂₇. Data are represented as Mean±SEM. Purple rectangles represent the nocturnal phase.

FIGS. 23A-23D show the impact of 89BIO-100 on the mean respiratory exchanges and related parameters during T₂₇. (FIG. 23A) Histograms of mean VO₂, adapted from the high-resolution recordings, (FIG. 23B) histograms of mean VCO₂, adapted from the high-resolution recordings, (FIG. 23C) histograms of mean EE, adapted from the profiles, (FIG. 23D) histograms of mean RER, adapted from the profiles of the Vehicle (n=7; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars) during T₂₇. Data are represented as Mean±SEM. * p<0.05, significantly different from the Vehicle group; ^{#} p<0.05, significantly different from the Liraglutide group.

FIGS. 24A-24D show the impact of 89BIO-100 on the total spontaneous activity during T₁ and T₂. (FIG. 24A) High-resolution recordings of total spontaneous activity during T₁, (FIG. 24B) high-resolution recordings of total spontaneous activity during T₂, (FIG. 24C) histograms of mean total spontaneous activity, adapted from the high-resolution recordings shown in FIG. 24A during the nocturnal phase (NP), the diurnal phase (DP) and the whole day (WD) on T₁ and (FIG. 24D) histograms of mean total spontaneous activity, adapted from the high-resolution recordings shown in FIG. 24B during the nocturnal phase (NP), the diurnal phase (DP) and the whole day (WD) on T₂ of Vehicle (n=6/8; grey curves/bars), Liraglutide (n=8/8; purple curves/bars), 89BIO-100 0.3 mg/kg (n=8/11; light blue curves/bars), 89BIO-100 1 mg/kg (n=8/11; blue curves/bars) and 89BIO-100 3 mg/kg (n=8/8; dark blue curves/bars). Data are represented as Mean+/−SEM. ^{#} p<0.05; ^{##} p<0.01, significantly different from the Liraglutide group. Purple rectangle represents the nocturnal phase.

FIGS. 25A-25C show the impact of 89BIO-100 on the body composition on H₇, T₁₃ and T₂₅. (FIG. 25A) Fat (g), (FIG. 25B) lean (g) and (FIG. 25C) fluid (g) masses of Vehicle (n=7; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars). Data are presented as mean+/−SEM. * p<0.05; ** p<0.01 significantly different from the Vehicle group; ^{$}p<0.05; ^{$$}p<0.01 significantly different from the 89BIO-100 0.3 mg/kg group, [symbol] p<0.05 significantly different from the 89BIO-100 1 mg/kg group.

FIGS. 26A-26C show the impact of 89BIO-100 on the variation in body composition between H₇, and T₂₅. (FIG. 26A) A Fat (g), (FIG. 26B) A lean (g) and (FIG. 26C) A fluid (g) masses of Vehicle (n=7; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars). Data are presented as mean+/−SEM. * p<0.05 significantly different from the Vehicle group; ^{$}p<0.05 significantly different from the 89BIO-100 0.3 mg/kg group.

FIG. 27A-27D show the impact of 89BIO-100 on semi-fasted blood glucose on H₇, T₃, T₁₆ and T₂₈. Blood glucose levels after 4 hours of fasting (FIG. 27A) at H₇, (FIG. 27B) at T₃, (FIG. 27C) at T₁₆ and (FIG. 27D) at T₂₈ of Vehicle (n=7; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars). Data are presented as mean+/−SEM. * p<0.05; ***p<0.001 significantly different from the Vehicle group; ^{#} p<0.05; ^{##} p<0.01; ^{###} p<0.001 significantly different from the Liraglutide group, $$ p<0.01 significantly different from the 89BIO-100 0.3 mg/kg group, ° ° p<0.001 significantly different from the 89BIO-100 1 mg/kg group.

FIGS. 28A-28F show the impact of 89BIO-100 on organ weights (g) on T₂₈. (FIG. 28A) Liver (g), (FIG. 28B) gastrocnemius muscle (g), (FIG. 28C) heart (g), (FIG. 28D) interscapular brown adipose tissue (iBAT; g), (FIG. 28E) epididymal white adipose tissue (eWAT; g) and (FIG. 28F) subcutaneous white adipose tissue (sWAT; g) of Vehicle (n=7; grey bars), Liraglutide (n=8; purple bars), 89BIO-100 0.3 mg/kg (n=11; light blue bars), 89BIO-100 1 mg/kg (n=11; blue bars) and 89BIO-100 3 mg/kg (n=8; dark blue bars). Data are presented as mean+/−SEM. * p<0.05; ** p<0.01; *** p<0.001 significantly different from the Vehicle group.

Among those benefits and improvements that have been disclosed, other objects and advantages of the present disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure are intended to be illustrative, and not restrictive.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, terms such as “comprising” “including,” and “having” do not limit the scope of a specific claim to the materials or steps recited by the claim.

As used herein, the term “consisting essentially of” limits the scope of a specific claim to the specified materials or steps and those that do not materially affect the basic and novel characteristic or characteristics of the specific claim.

As used herein, terms such as “consisting of” and “composed of” limit the scope of a specific claim to the materials and steps recited by the claim.

### Definitions

For the sake of clarity and readability, the following definitions are provided. Any technical feature mentioned for these definitions may be read on each and every embodiment of the the present disclosure. Additional definitions and explanations may be specifically provided in the context of these embodiments. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry, and nucleic acid chemistry and hybridization are those well known and commonly employed in the art. Standard techniques are used for nucleic acid and peptide synthesis. The techniques and procedures are generally performed according to conventional methods in the art and various general references (e.g., Sambrook et al., 1989, Molecular Cloning: A Laboratory Manual, 2d ed. Cold Spring Harbor Laboratory Press, Cold Spring Harbor, N.Y.), which are provided throughout this document.

Enzyme: Enzymes are catalytically active biomolecules that perform biochemical reactions such as the transfer of glycosyl moieties or modified glycosyl moieties from the respective glycosyl donors to an amino acid of FGF-21 or to another glycosyl moiety attached to the peptide.

Protein: A protein typically comprises one or more peptides or polypeptides. A protein is typically folded into a 3-dimensional form, which may be required for the protein to exert its biological function. The sequence of a protein or peptide is typically understood to be in the order, i.e. the succession of its amino acids.

Recombinant protein: The term “recombinant protein” refers to proteins produced in a heterologous system, that is, in an organism that naturally does not produce such a protein, or a variant of such a protein, i.e. the protein or peptide is “recombinantly produced”. Typically, the heterologous systems used in the art to produce recombinant proteins are bacteria (e.g., Escherichia (E.) coli), yeast (e.g., Saccharomyces (S.) cerevisiae) or certain mammalian cell culture lines.

Expression host: An expression host denotes an organism which is used for recombinant protein production. General expression hosts are bacteria, such as E. coli, yeasts, such as Saccharomyces cerevisiae or Pichia pastoris, or also mammal cells, such as human cells.

RNA, mRNA: RNA is the usual abbreviation for ribonucleic acid. It is a nucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA sequence.

DNA: DNA is the usual abbreviation for deoxyribonucleic acid. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers which are—by themselves—composed of a sugar moiety (deoxyribose), a base moiety and a phosphate moiety, and polymerized by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA-sequence. DNA may be single-stranded or double-stranded. In the double stranded form, the nucleotides of the first strand typically hybridize with the nucleotides of the second strand, e.g. by A/T-base-pairing and G/C-base-pairing.

Sequence of a nucleic acid molecule/nucleic acid sequence: The sequence of a nucleic acid molecule is typically understood to be in the particular and individual order, i.e. the succession of its nucleotides.

Sequence of amino acid molecules/amino acid sequence: The sequence of a protein or peptide is typically understood to be in the order, i.e. the succession of its amino acids.

Sequence identity: Two or more sequences are identical if they exhibit the same length and order of nucleotides or amino acids. The percentage of identity typically describes the extent, to which two sequences are identical, i.e. it typically describes the percentage of nucleotides that correspond in their sequence position to identical nucleotides of a reference sequence, such as a native or wild type sequence. For the determination of the degree of identity, the sequences to be compared are considered to exhibit the same length, i.e. the length of the longest sequence of the sequences to be compared. This means that a first sequence consisting of 8 nucleotides/amino acids is 80% identical to a second sequence consisting of 10 nucleotides/amino acids comprising the first sequence. In other words, in the context of the present disclosure, identity of sequences particularly relates to the percentage of nucleotides/amino acids of a sequence, which have the same position in two or more sequences having the same length. Gaps are usually regarded as non-identical positions, irrespective of their actual position in an alignment.

Newly introduced amino acids: “Newly introduced amino acids” denote amino acids which are newly introduced into an amino acid sequence in comparison to a native/wild type amino acid sequence. Usually by mutagenesis, the native amino acid sequence is changed in order to have a certain amino acid side chain at a desired position within the amino acid sequence. In the present disclosure, in particular the amino acid threonine is newly introduced into the amino acid sequence on the C-terminal side adjacent to a proline residue.

Functional group: The term is to be understood according to the skilled person's general understanding in the art and denotes a chemical moiety which is present on a molecule, in particular on the peptide or amino acid of the peptide or glycosyl residue attached to the peptide, and which may participate in a covalent or non-covalent bond to another chemical molecule, i.e. which allows e.g. the attachment of a glycosyl residue or PEG.

Native amino acid sequence: The term is to be understood according to the skilled person's general understanding in the art and denotes the amino acid sequence in the form of its occurrence in nature without any mutation or amino acid amendment by man. It is also called “wild-type sequence”. “Native FGF-21” or “wild-type FGF-21” denotes FGF-21 having the amino acid sequence as it occurs in nature, such as the (not mutated) amino acid sequence of human FGF-21 as depicted in SEQ ID NO: 1. See also the Sequence Listing, which presents the the sequences corresponding to SEQ ID NOs: to which reference is made herein. The presence or absence of an N-terminal methionine, which depends on the used expression host, usually does not change the status of a protein being considered as having its natural or native/wild-type sequence.

Mutated: The term is to be understood according to the skilled person's general understanding in the art. An amino acid sequence is called “mutated” if it contains at least one additional, deleted or exchanged amino acid in its amino acid sequence in comparison to its natural or native amino acid sequence, i.e. if it contains an amino acid mutation. Mutated proteins are also called mutants. In the present disclosure, a mutated FGF-21 peptide is particularly a peptide having an amino acid exchange adjacent to a proline residue on the C-terminal side of the proline residue. Thereby a consensus sequence for O-linked glycosylation is introduced into FGF-21 such that the mutant FGF-21 peptide comprises a newly introduced O-linked glycosylation side. Amino acid exchanges are typically denoted as follows: S¹⁷²T which means that the amino acid serine at position 172, such as in the amino acid sequence of SEQ ID NO: 1, is exchanged by the amino acid threonine.

Pharmaceutically effective amount: A pharmaceutically effective amount in the context of the present disclosure is typically understood to be an amount that is sufficient to induce a pharmaceutical effect.

Therapy/treatment: The term “therapy” refers to “treating” or “treatment” of a disease or condition, inhibiting the disease (slowing or arresting its development), providing relief from the symptoms or side-effects of the disease (including palliative treatment), and relieving the disease (causing regression of the disease).

Therapeutically effective amount: is an amount of a compound that is sufficient to treat a disease or condition, inhibit the disease or condition, provide relief from symptoms or side-effects of the disease, and/or cause regression of the disease or condition.

Half-life: The term “half-life”, as used herein in the context of administering a mutant FGF-21 peptide and/or conjugate thereof, is defined as the time required for the plasma concentration of a drug, i.e. of the mutant FGF-21 peptide and/or conjugate, in a subject to be reduced by one half.

O-linked glycosylation: “O-linked glycosylation” takes place at serine or threonine residues (Tanner et al., Biochim. Biophys. Acta. 906:81-91 (1987); and Hounsell et al, Glycoconj. J. 13:19-26 (1996)). In the present disclosure, O-linked glycosylation sites, which are amino acid motifs in the amino acid sequence of a peptide which are recognized by glycosyl transferases as attachment points for glycosyl residues, include the amino acid motif proline-threonine (PT) not present in the native/wild-type amino acid sequence. In particular, the threonine residue is newly introduced adjacent to a proline and on the C-terminal side of a proline residue. The glycosyl moiety is then attached to the —OH group of the threonine residue by the glycosyl transferase.

Newly introduced O-linked glycosylation side: “Newly introduced O-linked glycosylation side” denotes an O-linked glycosylation side which did not exist in the native or wild-type FGF-21 before introducing a threonine adjacent to and on the C-terminal side of a proline residue as described herein.

Adjacent: Adjacent denotes the amino acid immediately next to another amino acid in the amino acid sequence, either on the N-terminal or on the C-terminal side of the respective amino acid. In the present disclosure, e.g. the newly introduced threonine residue is adjacent to a proline residue on the C-terminal side of a proline residue.

Glycosyl moiety: A glycosyl moiety is a moiety consisting of one or more, identical or different glycosyl residues which links the mutant FGF-21 peptide to a polyethylene glycol (PEG), thereby forming a conjugate comprising a peptide, glycosyl moiety and PEG. The glycosyl moiety can be a mono-, di-, tri-, or oligoglycosyl moiety. The glycosyl moiety may comprise one or more sialic acid residues, one or more N-acetylgalactosamine (GalNAc) residues, one or more galactose (Gal) residues and others.

The glycosyl moiety may be modified, such as with a PEG or methoxy-PEG (m-PEG), an alkyl derivative of PEG.

Glycoconjugation: “Glycoconjugation”, as used herein, refers to the enzymatically mediated conjugation of a PEG-modified glycosyl moiety to an amino acid or glycosyl residue of a (poly)peptide, e.g. a mutant FGF-21 of the present disclosure. A subgenus of “glycoconjugation” is “glyco-PEGylation” in which the modifying group of the modified glycosyl moiety is PEG or m-PEG. The PEG may be linear or branched. Typically, a branched PEG has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched PEG can be represented in general form as R(-PEG-OX)ₘ in which R represents the core moiety, such as glycerol or pentaerythritol, X represents a capping group or an end group, and m represents the number of arms. The terms “glyco-PEG” and “glycosyl-PEG” are used interchangeably and denote a chemical moiety consisting of PEG or methoxy-PEG (mPEG or m-PEG), one or more glycosyl residues (or glycosyl moieties), and optionally a linker between PEG/methoxy-PEG and the glycosyl moieties, such as an amino acid, e.g. glycine. An example of a glycosyl-PEG/glyco-PEG moiety is PEG-sialic acid (PEG-Sia). It should be noted that the terms “glyco-PEG” and “glycosyl-PEG” as well as “PEG-sialic acid” and “PEG-Sia” as well as similar terms for glyco-PEG moieties may or may not include a linker between PEG and the glycosyl moiety or moieties, i.e. “PEG-sialic acid” encompasses e.g. PEG-sialic acid as well as PEG-Gly-sialic acid as well as mPEG-Gly-sialic acid.

Sequence motif: A sequence motif denotes a short amino acid sequence, such as that comprising only two amino acids, which is present at any possible position in a longer amino acid sequence, such as in the amino acid sequence of human FGF-21. Sequence motifs are e.g. denoted as P¹⁷²T which means that the proline at position 172 is followed C-terminally immediately by a threonine residue.

Sialic acid: The term “sialic acid” or “Sia” refers to any member of a family of nine-carbon carboxylated sugars. The most common member of the sialic acid family is N-acetyl-neuraminic acid (2-keto-5-acetamido-3,5-dideoxy-D-glycero-D-galacto-nonulopyranos-1-onic acid (often abbreviated as Neu5Ac, NeuAc, or NANA). A second member of the family is N-glycolylneuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261:11550-11557). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see e.g. Varki, Glycobiology 2:25-40 (1992)).

Pharmaceutically acceptable excipient: “Pharmaceutically acceptable” excipient includes any material, which when combined with the mutant FGF-21 peptide conjugate of the disclosure retains the conjugates' activity and is non-reactive with a subject's immune systems. Examples include, but are not limited to, any of the standard pharmaceutical excipients such as a phosphate buffered saline solution, water, salts, emulsions such as oil/water emulsion, and various types of wetting agents.

Pharmaceutical container: A “pharmaceutical container” is a container which is suitable for carrying a pharmaceutical composition and typically made of an inert material and sterile.

Administering: The term “administering” means oral administration, inhalation, administration as a suppository, topical contact, intravenous, intraperitoneal, intramuscular, intralesional, intranasal or subcutaneous administration, or the implantation of a slow-release device e.g., a mini-osmotic pump, to the subject.

Administration is by any route including parenteral, and transmucosal (e.g. oral, nasal, vaginal, rectal, or transdermal). Parenteral administration includes e.g. intravenous, intramuscular, intraarteriole, intradermal, subcutaneous, intraperitoneal, intraventricular and intracranial. Other modes of delivery include, but are not limited to, the use of liposomal formulations, intravenous infusion, transdermal patches, etc.

Diabetes and diabetes related diseases: “Diabetes” is a well-known and well-characterized disease often referred to as diabetes mellitus. The term describes a group of metabolic diseases in which the person has high blood glucose levels (blood sugar), either because insulin production is inadequate, or because the body's cells do not respond properly to insulin, or both. Patients with high blood sugar will typically experience polyuria (frequent urination), they will become increasingly thirsty (polydipsia) and hungry (polyphagia). “Diabetes related diseases” are diseases characterized by the same symptoms such as obesity, polyuria, polydipsia and polyphagia.

Diabetes type 2: “Diabetes type 2” is the most common form of diabetes/diabetes mellitus. Diabetes type 2 most commonly develops in adulthood and is more likely to occur in people who are overweight and physically inactive. Unlike type 1 diabetes, which currently cannot be prevented, many of the risk factors for type 2 diabetes can be modified. The International Diabetes Foundation lists four symptoms that signal the need for diabetes testing: a) frequent urination, b) weight loss, c) lack of energy and d) excessive thirst. Insulin resistance is usually the precursor to diabetes type 2 a condition in which more insulin than usual is needed for glucose to enter the cells. Insulin resistance in the liver results in more glucose production while resistance in peripheral tissues means glucose uptake is impaired.

Characteristics associated with pre-diabetes or diabetes are understood in the art. For example, a fasting blood sugar level ranging from 100 to 125 mg/dL (5.6 to 6.9 mmol/L) is characteristic of pre-diabetes in a human subject. A fasting blood sugar level of 126 mg/dL (7 mmol/L) or higher on two separate tests, is diagnostic for diabetes. When using an oral glucose tolerance test, a reading of more than 200 mg/dL (11.1 mmol/L) after two hours indicates diabetes.

The oral glucose tolerance test (OGTT) is a standard test used to determine if a person has diabetes. In, for example, a two-hour test, 75-gram OGTT is typically used to test for diabetes. A fasting lab draw of blood is taken to test fasting glucose levels in a subject. Thereafter, the subject consumes a syrupy glucose solution (˜8 ounces) that contains 75 grams of sugar. One and two hours after consuming the syrupy glucose solution, blood is drawn and assayed to determine blood sugar levels post-consumption.

Diagnosis of the following can be made in accordance with standard practice as set forth in Table 1.

TABLE 1
presents ranges of blood sugar levels post-consumption that serves as positive indicators of
prediabetes, diabetes, or gestational diabetes.

| When blood is drawn | For prediabetes | For diabetes | For gestational diabetes | |
|---|---|---|---|---|
| Fasting | 100-125 mg/dL | 126 mg/dL or greater | Greater than 92 | mg/dL |
| After 1 hour | | | Greater than 180 | mg/dL |
| After 2 hours | 140-199 mg/dL | 200 mg/dL or greater | Greater than 153 | mg/dL |

If a subject's results after one hour are equal to or greater than 135 or 140 mg/dL, the attending medical practitioner will recommend proceeding to the second step of the test, which involves consuming 100 grams of sugar. If two of the blood sugar levels post-consumption are higher than those listed in Table 2, the subject from whom the blood was drawn is diagnosed as having gestational diabetes.

TABLE 2
presents ranges of blood sugar levels
post-consumption that serves as positive
indicator of gestational diabetes.

| When blood | | |
|---|---|---|
| is drawn | Diagnostic levels | |
| Fasting | Greater than 95 | mg/dL |
| After 1 hour | Greater than 180 | mg/dL |
| After 2 hours | Greater than 155 | mg/dL |
| After 3 hours | Greater than 140 | mg/dL |

HOMA-IR is, for example, is an indicator of the presence and extent of insulin resistance in a subject. It is an accurate indicator of the dynamic between baseline (fasting) blood sugar and insulin levels responsive thereto. It is referred to as an insulin resistance calculator. For humans, a healthy range is 1.0 (0.5-1.4). Less than 1.0 indicates that a subject is insulin-sensitive, which is ideal; above 1.9 indicates that a subject is exhibiting early insulin resistance; above 2.9 indicates that a subject is exhibiting significant insulin resistance. HOMA-IR blood code calculation is determined as follows: insulin uIU/mL (mU/L)×glucose (mg/dL)=HOMA-IR. The calculation requires U.S. standard units. To convert from international SI units: for insulin: pmol/L to uIU/mL, divide (±) by 6; for glucose: mmol/L to mg/dL, multiply (X) by 8.

Measuring HbA1C is considered a standard assay for measuring glycemic index of a subject over a long duration. It is, therefore, a stable indicator of glycemic index, reflecting glucose levels over the course of approximately the last 3-4 months.

Accordingly, a subject who has diabetes (e.g., diabetes type 2) may be defined by the percent HbA1C determined in a suitable assay.

For a healthy person without diabetes, the normal range for the hemoglobin A1c level is between 4% and 5.6%. Hemoglobin A1c levels between 5.7% and 6.4% indicate that a person has a higher chance of developing diabetes. Levels of 6.5% or higher indicate that a person has diabetes.

Metabolic syndrome: a defined cluster of risk factors (biochemical and physiological changes) that are associated with the development of type 2 diabetes and cardiovascular disease. The National Institutes of Health guidelines define metabolic syndrome as having three or more of the following traits:
Large waist: A waistline that measures at least 35 inches (89 centimeters) for women and 40 inches (102 centimeters) for men
If a subject's BMI is greater than 30 kg/m², central obesity can be assumed and waist circumference does not need to be measured.
High triglyceride level: 150 milligrams per deciliter (mg/dL), or 1.7 millimoles per liter (mmol/L), or higher of this type of fat found in blood
Reduced “good” or HDL cholesterol: Less than 40 mg/dL (1.04 mmol/L) in men or less than 50 mg/dL (1.3 mmol/L) in women of high-density lipoprotein (HDL) cholesterol
Increased blood pressure: 130/85 millimeters of mercury (mm Hg) or higher
Elevated fasting blood sugar: 100 mg/dL (5.6 mmol/L) or higher

Accordingly, a subject having three or more of the above traits or characteristics is diagnosed as having metabolic syndrome.

Non-alcoholic steatohepatitis (NASH): a condition where fat is deposited in the liver with subsequent liver damage and inflammation. It is typically associated with symptoms characteristic of metabolic syndrome. Liver biopsy is generally considered the gold standard for diagnosing NASH. Alternative, noninvasive diagnostic methods include imaging-based techniques, such as: ultrasound, magnetic resonance imaging, transient elastography, ultrasound elastography, and magnetic resonance elastography.

Routine blood tests such as those for detecting levels of, for example, platelets and liver enzyme levels [aspartate aminotransferase (AST) and alanine transaminase (ALT)] are also useful diagnostic indicators, particularly when used in conjunction with other test diagnostic tests for NASH. AST and ALT are mildly elevated with an ALT predominance and usually not exceeding 250 IU/L. The mean ALT and AST levels from a large cohort of biopsy-proven NASH patients were recently found to be 69 and 51 IU/L, respectively.

FGF-21 is an endocrine hormone that is naturally found as a monomeric non-glycosylated protein. Together with FGF-19 and FGF-23, FGF-21 belongs to the endocrine-acting sub-family, while the remaining 18 mammalian FGF ligands are grouped into five paracrine-acting sub-families. Endocrine-acting FGFs, in contrast to paracrine-acting FGFs, exhibit only low affinity for heparin-sulfate and are thus able to enter the blood circulation. Accordingly, endocrine FGFs are able to regulate metabolic processes, such as bile acid homeostasis, hepatic glucose and protein metabolism (FGF-19), glucose and lipid metabolism (FGF-21) and vitamin D and phosphate homeostasis (FGF-23).

Natural FGF-21 has a comparatively short half-life in vivo, with a reported circulating half-life ranging from 0.5 to 4 hours in rodents and non-human primates, which limits its clinical applicability. The half-life of recombinant human FGF-21 is 1-2 hours. To improve pharmacokinetic properties of FGF-21, various half-life extension strategies have been developed.

See also WO2019/043457, the entire content of which is incorporated herein in its entirety.

Abbreviations used herein include: PEG, poly(ethyleneglycol); PPG, poly(propyleneglycol); Ara, arabinosyl; Fru, fructosyl; Fuc, fucosyl; Gal, galactosyl; GalNAc, N-acetylgalactosaminyl; Glc, glucosyl; GlcNAc, N-acetylglucosaminyl; Man, mannosyl; ManAc, mannosaminyl acetate; Xyl, xylosyl; NeuAc, sialyl or N-acetylneuraminyl; Sia, sialyl or N-acetylneuraminyl; and derivatives and analogues thereof.

### PEGylation

One method to prolong a protein's half-life is the attachment of one or more PEG moieties to the protein, which attachment increases the protein's biophysical solubility and stability in general. This approach has proven to be of particular value with respect to increasing the therapeutic half-life of proteins having properties suitable for treating subjects in need thereof. Native FGF-21, however, lacks a specific protein PEGylation site. Chemical PEGylation absent a specific protein PEGylation site is not site-specific and typically results in the generation of an inhomogeneous product population requiring extensive purification to achieve a homogeneous and high purity product—a prerequisite for market approval as a pharmaceutical composition. Accordingly, site-specific PEGylation of FGF-21 is desirable for generating site-specific PEGylated FGF-21 peptides having improved half-life and good biological activity.

Enzyme-based syntheses have the advantages of regioselectivity and stereoselectivity. Moreover, enzymatic syntheses may be performed with unprotected substrates. One possible method to attach PEG residues site-specifically to a protein is glycoPEGylation. In glycoPEGylation, a PEG moiety may be transferred to an amino acid or glycosyl residue attached to an amino acid of the protein or peptide using a glycosyltransferase. The general final structure is protein—glycosyl moiety—optional further linker—PEG. A more particular final structure is protein—(N-, C- or internal) amino acid of the protein—one or more glycosyl residues—optional linker (e.g., amino acid linker)—linear or branched PEG moiety of various lengths, wherein the glycosyl moiety may comprise one or more glycosyl residues. The one or more glycosyl residues comprising at least part of the structure linking the protein to the PEG moiety may be any possible glycosyl residue. A diverse array of methods for glycoPEGylating proteins are known in the art and are described in detail herein below.

In protein PEGylation, the larger the conjugated PEG moiety, the longer the expected half-life of a PEG-conjugated protein. This is due to the relatively enhanced ability of larger PEG moieties to protect conjugated proteins from proteases present in the blood stream. Large PEG moieties confer a larger effective radius to a PEG-conjugated protein than smaller PEG moieties. Larger proteins are also degraded in and removed from the blood stream more slowly than smaller proteins because they enter the kidney more slowly or are prevented from entering the kidney completely. Accordingly, skilled persons favor PEGylation processes that call for attaching a longer PEG residue of higher molecular weight (e.g., ≥30 kDa PEG), a higher number of PEG residues in total, and/or more highly branched PEG residues to a protein in order to create a PEGylated protein having superior properties relative to the same protein conjugated to a shorter/smaller PEG moiety.

A considerable disadvantage associated with pegylation is, however, the potential for steric hindrance whereby a conjugated PEG moiety physically blocks an active site of the protein that is important or essential for protein activity. For example, a PEG moiety may specifically block a receptor binding site of a protein for its receptor, which in turn, leads to a significant and detrimental loss in protein activity. To avoid such potential inhibitory effects of pegylation, persons skilled in the art avoid attaching PEG near amino acids involved in receptor binding.

With respect to FGF-21, the C-terminus is critical for β-Klotho binding and the N-terminus is important for FGFR activation. Moreover, in silico modeling of FGF-21 based on the crystal structures of other FGF-21 family proteins and in vitro potency assays demonstrated that PEGylation of amino acid residues located in the putative receptor binding domains were inactive, while PEGylation at distal sites produced the most active analogs. Furthermore, greater than 100-fold loss of potency was observed in a cell based potency assay when a PEG moiety was placed at position 180 in FGF-21. Fusion of FGF-21 to the Fc portion of an antibody was also examined, and fusion at the C-terminus of FGF-21 produced a much weaker analog than fusion at the N-terminus. In contrast, N-terminally PEGylated FGF-21 has been generated and shown to be biologically active. Based on knowledge in the field, therefore, a skilled person would avoid PEGylation close to the C-terminus of FGF-21 given the role this region of the protein plays in binding and signaling. A plurality of mutant Fibroblast Growth Factor-21 (FGF-21) peptide conjugates are described herein, each comprising

i) a mutant FGF-21 peptide comprising at least one threonine (T) residue adjacent to at least one proline (P) residue on the C-terminal side of said at least one proline residue, thereby forming at least one O-linked glycosylation site which does not exist in the corresponding native FGF-21, wherein the corresponding native FGF-21 has an amino acid sequence that is at least 95% identical to SEQ ID NO: 1, and

ii) a 20 kDa polyethylene glycol (PEG), wherein said 20 kDa PEG is covalently attached to said mutant FGF-21 peptide at said at least one threonine residue via at least one glycosyl moiety.

In a particular embodiment, the mutant FGF-21 peptide conjugate comprises a mutant FGF-21 peptide comprising the amino acid sequence Proline-Threonine (PT). In particular embodiments thereof, the mutant FGF-21 peptide comprises at least one amino acid sequence selected from the group consisting of P172T, P156T, P5T, P3T, P9T, P50T, P61T, P79T, P91T, P116T, P129T, P131T, P134T, P139T, P141T, P144T, P145T, P148T, P150T, P151T, P158T, P159T, P166T, P178T and combinations thereof, wherein the positions of proline and threonine are based on the amino acid sequence as depicted in SEQ ID NO: 1. In a more particular embodiment, the mutant FGF-21 peptide comprises at least one amino acid sequence selected from the group consisting of P172T, P156T, P5T and combinations thereof, particularly consisting of P172T, P156T and combinations thereof, wherein the positions of proline and threonine are based on the amino acid sequence as depicted in SEQ ID NO: 1. In a still more particular embodiment, the proline residue is located between amino acid 145 and the C-terminus of the mutant FGF-21 peptide, wherein the position of amino acid 145 is based on the amino acid sequence as depicted in SEQ ID NO: 1.

In another particular embodiment, the mutant FGF-21 peptide comprises the amino acid sequence P172T, wherein the positions of proline and threonine are based on the amino acid sequence as depicted in SEQ ID NO: 1.

In another particular embodiment, the mutant FGF-21 peptide comprises the mutations S173T and R176A, wherein the positions of the amino acids Serine (S) and Arginine (R) are based on the amino acid sequence as depicted in SEQ ID NO: 1, particularly the mutant FGF-21 peptide comprising the amino acid sequence as depicted in SEQ ID NO: 2.

In another particular embodiment, the mutant FGF-21 peptide comprises the mutation Q157T, wherein the position of the amino acid Q is based on the amino acid sequence as depicted in SEQ ID NO: 1, particularly the mutant FGF-21 peptide comprising the amino acid sequence as depicted in SEQ ID NO: 4.

In another particular embodiment, the mutant FGF-21 peptide comprises the mutation D6T, wherein the position of the amino acid D is based on the amino acid sequence as depicted in SEQ ID NO: 1, particularly the mutant FGF-21 peptide comprising the amino acid sequence as depicted in SEQ ID NO: 5.

In other particular embodiments, the mutant FGF-21 peptide comprises an amino acid sequence selected from the group consisting of SEQ ID NOs: 2 to 28, particularly an amino acid sequence selected from the group consisting of SEQ ID NOs: 2 to 5, more particularly an amino acid sequence selected from the group consisting of SEQ ID NOs: 2 to 4, and most particularly the mutant FGF-21 peptide comprises the amino acid sequence as depicted in SEQ ID NO: 2.

In other particular embodiments, the mutant FGF-21 peptide conjugate comprises at least one glycosyl moiety comprising N-acetylgalactosamine (GalNAc), galactose (Gal) and/or sialic acid (Sia). In a particular embodiment thereof, the at least one glycosyl moiety comprises the structure -GalNAc-Sia-.

In other particular embodiments, the mutant FGF-21 peptide conjugate comprises a 20 kDa PEG moiety which is attached to the at least one glycosyl moiety via an amino acid residue, particularly glycine (Gly). In an even more particular embodiment, the mutant FGF-21 peptide conjugate comprises the structure -GalNAc-Sia-Gly-PEG(20 kDa). Still more particularly, the mutant FGF-21 peptide conjugate comprises the structure:

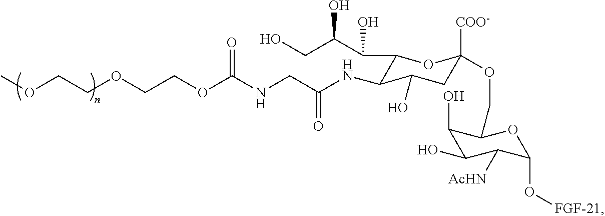

wherein n is an integer selected from 450 to 460.

In other particular embodiments, the mutant FGF-21 peptide conjugate comprises a 20 kDa PEG which is a linear or branched PEG, particularly a linear PEG. Still more particularly, the 20 kDa PEG is a 20 kDa methoxy-PEG.

Also encompassed herein is a pharmaceutical composition comprising at least one mutant FGF-21 peptide conjugate described herein and a pharmaceutically acceptable carrier. In a particular embodiment, the mutant FGF-21 peptide conjugate is present in a concentration in the range from 0.1 mg/mL to 50 mg/mL, particularly from 1 mg/mL to 45 mg/mL, more particularly from 10 mg/mL to 40 mg/mL, most particularly in a concentration of 26±4 mg/mL. The buffering agent may be a Tris buffer. The buffering agent may be present in a concentration from 1 mM to 100 mM, particularly from 2 mM to 75 mM, more particularly from 5 mM to 50 mM, even more particularly from 10 mM to 25 mM, most particularly of 16±2 mM. The pH may be in the range from 6.0 to 8.5, particularly from 6.5 to 8.0, more particularly from 6.75 to 8.0, and most particularly is 7.5±0.3. The pharmaceutical composition may further comprise a salt, particularly an inorganic salt, more particularly NaCl. The pharmaceutical composition may comprise a salt which is present in a concentration from 30 mM to 200 mM, particularly from 40 mM to 150 mM, more particularly from 50 mM to 100 mM, most particularly of 56±2 mM. The pharmaceutical composition may further comprise a tonicity modifying agent. Suitable tonicity modifying agents include glycerol, amino acids, sodium chloride, proteins, or sugars and sugar alcohols, particularly the tonicity modifying agent is a sugar, and more particularly the tonicity modifying agent is sucrose. The tonicity modifying agent is present in a concentration of 50 mM to 200 mM, more particularly in a concentration of 100 mM to 175 mM, even more particularly is present in a concentration of 135 mM to 160 mM, and most particularly in a concentration of 150±2 mM. The pharmaceutical composition may further comprise a surfactant, particularly a non-ionic surfactant. The surfactant or non-ionic surfactant may be a polysorbate-based non-ionic surfactant, particularly polysorbate 20 or polysorbate 80, and more particularly polysorbate 20. The surfactant or non-ionic surfactant may be present in a concentration of 0.01 mg/mL to 1 mg/mL, particularly in a concentration of 0.05 to 0.5 mg/mL and more particularly in a concentration of 0.2±0.02 mg/mL.

In a particular embodiment, the pharmaceutical composition comprises 0.1 mg/mL to 50 mg/mL of mutant FGF-21 peptide conjugate, 1 mM to 100 mM buffering agent, particularly Tris buffer, 30 mM to 200 mM mM salt, particularly NaCl, 50 mM to 200 mM tonicity modifying agent, particularly sucrose, and 0.01 mg/mL to 1 mg/mL surfactant or non-ionic surfactant, particularly polysorbate 20, and has a pH of 6.0 to 8.5. A pharmaceutical container comprising at least one of the mutant FGF-21 peptide conjugates described herein and/or a pharmaceutical composition comprising same are also encompassed herein. Suitable pharmaceutical containers include, without limitation, a syringe, vial, infusion bottle, ampoule, carpoule, a syringe equipped with a needle protection system, and a carpoule within an injection pen.

Also encompassed herein is a method of producing the mutant FGF-21 peptide conjugate, comprising the steps of:

(1) recombinantly producing the mutant FGF-21 peptide in an expression host; and

(2) enzymatically attaching to the mutant FGF-21 peptide of step (1) a PEG-glycosyl moiety, wherein the PEG has 20 kDa, thereby forming the mutant FGF-21 peptide conjugate. In a particular embodiment, the expression host is Escherichia coli. In a more particular embodiment, step (2) comprises a step (2a) of contacting the mutant FGF-21 peptide with a GalNAc donor and a GalNAc transferase under conditions suitable to transfer GalNAc from the GalNAc donor to the at least one threonine residue of the mutant FGF-21 peptide. In a still more particular embodiment, the GalNAc donor is UDP-GalNAc. In another particular embodiment, the GalNAc transferase is MBP-GalNAcT2. In another particular embodiment, step (2) further comprises a step (2b) of contacting the product of step (1) or of step (2a), if present, with a 20 kDa PEG-Sia donor and a sialyltransferase under conditions suitable to transfer 20 kDa PEG-Sia from the 20 kDa PEG-Sia donor to the at least one threonine residue of the mutant FGF-21 peptide or to the GalNAc at the mutant FGF-21 peptide if step (2a) is present. In a more particular embodiment, the 20 kDa PEG-Sia donor is 20 kDa PEG-Sia-CMP. In a still more particular embodiment, the sialyltransferase is ST6GalNAc1. In a still further particular embodiment, the 20 kDa PEG-Sia donor comprises the structure

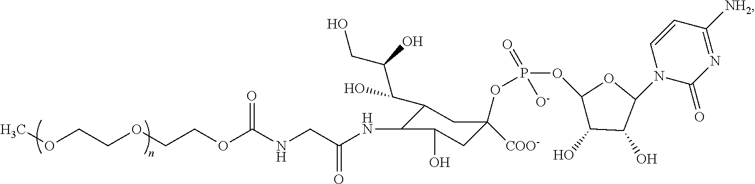

wherein n is an integer selected from 450 to 460.

In another particular embodiment, the method further comprises a step (3), after step (1) and prior to step (2), of purifying the mutant FGF-21 peptide after recombinant production. In a more particular embodiment, the method further comprises a step (4), after step (2), of purifying the mutant FGF-21 peptide conjugate formed in step (2). In another particular embodiment, the method, wherein step (3) comprises subjecting the mutant FGF-21 peptide and/or step (4) comprises subjecting the mutant FGF-21 peptide conjugate, the method may comprise ion exchange chromatography, affinity chromatography, filtration or combinations thereof. More particularly, wherein the step of purifying comprises one or more steps of ion exchange chromatography, it particularly comprises two steps of ion exchange chromatography. In another particular embodiment, wherein the ion exchange chromatography is an anion exchange chromatography, it is more particularly a strong anion exchange chromatography. More particularly, wherein the anion exchange chromatography employs a member, it is selected from the group consisting of a hydrophilic polyvinyl ether base matrix, polystyrene/divinyl benzene polymer matrix, trimethylammoniumethyl (TEAE), diethylaminoethanol (DEAE), agarose, a quaternary ammonium (Q) strong anion exchange chromatography and combinations thereof. In another particular embodiment, wherein in step (3) two anion exchange chromatography steps are performed, such steps use a hydrophilic polyvinyl ether base matrix. Still more particularly, wherein in step (4) quaternary ammonium (Q) strong anion exchange chromatography steps are performed, two quaternary ammonium (Q) strong anion exchange chromatography steps are performed. More particularly, wherein arginine is added in step (2) and/or, if present, in step (3), it is particularly at least 400 mM arginine. In a more particular embodiment, the method may further comprise a step (5), after step (3) and prior to step (2), of endotoxin removal, wherein the product of step (3) is filtered using an endotoxin removal filter.

In a further aspect, a mutant FGF-21 peptide conjugate obtainable by the above methods is encompassed, as are pharmaceutical compositions thereof that further comprise a pharmaceutically acceptable excipient or carrier.

In another aspect, a method for promoting weight loss in a subject in need thereof that does not have symptoms associated with diabetes, particularly diabetes type 2, NASH and/or metabolic syndrome) is presented, the method comprising administering to the subject in need thereof a therapeutically effective amount of a mutant FGF-21 peptide conjugate described herein or a pharmaceutical composition comprising at least one of the mutant FGF-21 peptide conjugates described herein. In a particular embodiment, the subject in need thereof is a human subject.

### Methods for Glycosylation and Glycoconjugation of FGF-21 Peptides

Post-expression in vitro modification of peptides and proteins is commonly used to produce glycopeptides and glycoproteins. A diverse array of enzymes that transfer saccharide donor moieties is available, thereby making in vitro enzymatic synthesis of glycoconjugates with custom designed glycosylation patterns and glycosyl structures possible. See, for example, U.S. Pat. Nos. 5,876,980; 6,030,815; 5,728,554; 5,922,577; 9,200,049; and 10,407,479; and published patent applications WO 98/31826; WO 01/88117; WO 03/031464; WO 03/046150; WO 03/045980; WO 03/093448; WO 04/009838; WO 05/089102; WO 06/050247; WO 12/016984; US2002/142370; US2003/040037; US2003/180835; US2004/063911; US2003/207406; and US2003/124645, each of which is incorporated herein by reference.

Due to the versatility of the enzymes and methods available for adding and/or modifying glycosyl residues on a peptide, the glycosyl linking groups can have substantially any structure. Accordingly, glycosyl linking groups can comprise virtually any mono- or oligo-saccharide. The glycosyl linking groups can be attached to an amino acid either through the side chain or through the peptide backbone. Alternatively, the glycosyl linking groups can be attached to the peptide through a saccharyl moiety, which moiety can be a portion of an O-linked or N-linked glycan structure on the peptide.

In accordance with the above, the present inventors set out to make conjugates of glycosylated mutant FGF-21, which have glycosylation sites that do not exist in the corresponding wild-type FGF-21 sequence. Such conjugates were formed by the enzymatic attachment of a modified sugar to the glycosylated FGF-21 peptide. The modified sugar, when interposed between the peptide and the modifying group on the sugar may be referred to herein as “a glycosyl linking group.” Taking advantage of the exquisite selectivity of enzymes, such as glycosyltransferases, the present inventors generated mutant FGF-21 peptides having a desired group at one or more specific locations. More particularly, the present inventors used glycosyltransferases to attach modified sugars to carbohydrate moieties on mutant FGF-21 glycopeptides.

### FGF-21 Conjugates

In another aspect, exemplary conjugates of a modified sugar and a mutant FGF-21 peptide are presented. More particularly, mutant FGF-21 peptide conjugates were made comprising a mutant FGF peptide and at least one modified sugar, wherein the first of the at least one modified sugars is linked to an amino acid of the peptide through a glycosyl linking group. As described herein, the amino acid to which the glycosyl linking group is attached is mutated to create a site recognized by the glycosyltransferase. In some embodiments, a mutant FGF-21 peptide conjugate can comprise a mutant FGF-21 peptide and a glycosyl group attached to the mutated amino acid residue of the mutant FGF-21 peptide.

In some embodiments, the glycosyl group is an intact glycosyl linking group. In some embodiments, the glycosyl group further comprises a modifying group. In some embodiments, the modifying group is a non-glycosidic modifying group. In some embodiments, the modifying group does not include a naturally occurring saccharide moiety.

### Modified Sugars

In some embodiments, mutant FGF-21 peptides are reacted with a modified sugar, thus forming a peptide conjugate. A modified sugar comprises a “sugar donor moiety” as well as a “sugar transfer moiety”. The sugar donor moiety is any portion of the modified sugar that will be attached to the peptide, either through a glycosyl moiety or amino acid moiety, as a conjugate described herein. The sugar donor moiety includes those atoms that are chemically altered during their conversion from the modified sugar to the glycosyl linking group of the mutant FGF-21 peptide conjugate. The sugar transfer moiety is any portion of the modified sugar that will be not be attached to the peptide as a conjugate described herein.

For modified sugars described herein, the saccharyl moiety may be a saccharide, a deoxy-saccharide, an amino-saccharide, or an N-acyl saccharide. The term “saccharide” and its equivalents, “saccharyl,” “sugar,” and “glycosyl” refer to monomers, dimers, oligomers and polymers. The sugar moiety is also functionalized with a modifying group. The modifying group is conjugated to the saccharyl moiety, typically, through conjugation with an amine, sulfhydryl or hydroxyl, e.g., primary hydroxyl, moiety on the sugar. In some embodiments, the modifying group is attached through an amine moiety on the sugar, e.g., through an amide, a urethane or a urea that is formed through the reaction of the amine with a reactive derivative of the modifying group.

Any saccharyl moiety can be utilized as the sugar donor moiety of the modified sugar. The saccharyl moiety can be a known sugar, such as mannose, galactose or glucose, or a species having the stereochemistry of a known sugar. The general formulae of these modified sugars are:

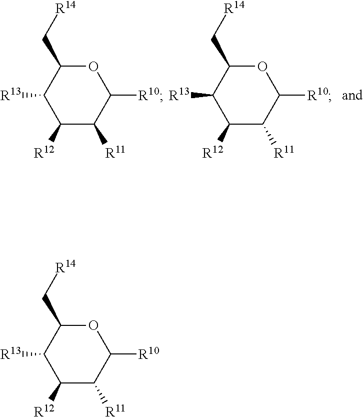

Other saccharyl moieties that are useful in methods described herein include, but are not limited to fucose and sialic acid, as well as amino sugars such as glucosamine, galactosamine, mannosamine, the 5-amine analogue of sialic acid and the like. The saccharyl moiety can be a structure found in nature or it can be modified to provide a site for conjugating the modifying group. For example, in one embodiment, the modified sugar provides a sialic acid derivative in which the 9-hydroxy moiety is replaced with an amine. The amine is readily derivatized with an activated analogue of a selected modifying group. Examples of modified sugars useful in methods described herein are presented in PCT Patent Application No. PCT/US05/002522, which is incorporated herein by reference in its entirety.

A further embodiment utilizes modified sugars in which the 6-hydroxyl position is converted to the corresponding amine moiety, which bears a linker-modifying group cassette such as those set forth above. Exemplary glycosyl groups that can be used as the core of these modified sugars include Gal, GalNAc, Glc, GlcNAc, Fuc, Xyl, Man, and the like. A representative modified sugar according to this embodiment is set forth below:

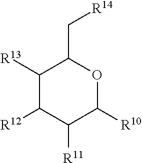

in which R¹¹-R¹⁴ are members independently selected from H, OH, C(O)CH₃, NH, and NH C(O)CH₃. R¹⁰ is a link to, e.g., another glycosyl residue (—O-glycosyl). R¹¹ is OR¹, NHR¹ or NH-L-R¹. R¹ and NH-L-R¹ are as described herein.

In a still further embodiment, the glycosyl groups used as the core of modified sugars in which the 6-hydroxyl position is converted to the corresponding amine moiety include Gal and/or GalNAc.

### Glycosyl Linking Groups

In some embodiments, mutant FGF-21 peptide conjugates comprising a modified sugar described herein and a mutant FGF peptide are presented. In this embodiment, the sugar donor moiety (such as the saccharyl moiety and the modifying group) of the modified sugar becomes a “glycosyl linking group”. The “glycosyl linking group” can alternatively refer to the glycosyl moiety which is interposed between the peptide and the modifying group.

The exemplary embodiments that follow are illustrated by reference to the use of selected derivatives of furanose and pyranose. Those of skill in the art will appreciate that the structures and compositions set forth are generally applicable across the genus of glycosyl linking groups and modified sugars. The glycosyl linking group can, therefore, comprise virtually any mono- or oligo-saccharide.

In some embodiments, methods described herein utilize a glycosyl linking group that has the formula:

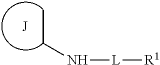

in which J is a glycosyl moiety, L is a bond or a linker and R¹ is a modifying group, e.g., a polymeric modifying group. Exemplary bonds are those that are formed between an NH₂ moiety on the glycosyl moiety and a group of complementary reactivity on the modifying group. For example, when R¹ includes a carboxylic acid moiety, this moiety may be activated and coupled with the NH₂ moiety on the glycosyl residue affording a bond having the structure NHC(O)R¹. J is preferably a glycosyl moiety that is “intact”, not having been degraded by exposure to conditions that cleave the pyranose or furanose structure, e.g. oxidative conditions, e.g., sodium periodate.

Exemplary linkers include alkyl and heteroalkyl moieties. The linkers include linking groups, for example acyl-based linking groups, e.g., —C(O)NH—, —OC(O)NH—, and the like. The linking groups are bonds formed between components of the conjugates, e.g., between the glycosyl moiety and the linker (L), or between the linker and the modifying group (R¹). Other exemplary linking groups are ethers, thioethers and amines.

For example, in one embodiment, the linker is an amino acid residue, such as a glycine residue. The carboxylic acid moiety of the glycine is converted to the corresponding amide by reaction with an amine on the glycosyl residue, and the amine of the glycine is converted to the corresponding amide or urethane by reaction with an activated carboxylic acid or carbonate of the modifying group.

An exemplary species of NH-L-R¹ has the formula: —NH{C(O)(CH₂)ₐNH}ₛ{C(O)(CH₂)_{b}(OCH₂CH₂)_{c}O(CH₂)_{d}NH}ₜR¹, in which the indices s and t are independently 0 or 1. The indices a, b and d are independently integers from 0 to 20, and c is an integer from 1 to 2500. Other similar linkers are based on species in which an NH moiety is replaced by another group, for example, S, O or CH₂.

As is understood in the art, one or more of the bracketed moieties corresponding to indices s and t can be replaced with a substituted or unsubstituted alkyl or heteroalkyl moiety.

More particularly, compounds described herein may comprise NH-L-R′, wherein NH-L-R′ is: NHC(O)(CH₂)ₐNHC(O)(CH₂)_{b}(OCH₂CH₂)_{c}O(CH₂)_{d}NHR¹, NHC(O)(CH₂)_{b}(OCH₂CH₂)_{c}O(CH₂)_{d}NHR¹, NHC(O)O(CH₂)_{b}(OCH₂CH₂)_{c}O(CH₂)_{d}NHR¹, NH(CH₂)ₐNHC(O)(CH₂)_{b}(OCH₂CH₂)_{c}O(CH₂)_{d}NHR¹, NHC(O)(CH₂)ₐNHR¹, NH(CH₂)ₐNHR¹, and NHR¹. In these formulae, the indices a, b and d are independently selected from the integers from 0 to 20, preferably from 1 to 5. The index c is an integer from 1 to about 2500.

In some embodiments, c is selected such that the PEG moiety is approximately 1 kD, 5 kD, 10, kD, 15 kD, 20 kD, 25 kD, 30 kD, 35 kD, 40 kD, 45 kD, 50 kD, 55 kD, 60 kD or 65 kD.

In a more particular embodiment, the c is selected such that the PEG moiety ranges from 15-25 kD, 16-25 kD, 17-25 kD, 18-25 kD, 19-25 kD, 20-25 kD, 21-25 kD, 22-25 kD, 23-25 kD, 24-25 kD, 15-20 kD, 16-20 kD, 17-20 kD, 18-20 kD, 19-20 kD, 20-30 kD, 21-30 kD, 22-30 kD, 23-30 kD, 24-30 kD, 25-30 kD, 26-30 kD, 27-30 kD, 28-30 kD, 29-30 kD. In a still more particular embodiment, the c is selected such that the PEG moiety is 20 kD, 22 kD, 23 kD, 24 kD, 25 kD, 26 kD, 27 kD, 28 kD, 29 kD, or 30 kD.

For the purposes of clarity, the glycosyl linking groups in the remainder of this section are based on a sialyl moiety. However, one of skill in the art will recognize that another glycosyl moiety, such as mannosyl, galactosyl, glucosyl, or fucosyl, could be used in place of the sialyl moiety.

In some embodiments, the glycosyl linking group is an intact glycosyl linking group, in which the glycosyl moiety or moieties forming the linking group are not degraded by chemical (e.g., sodium metaperiodate) or enzymatic (e.g., oxidase) processes. Selected conjugates of the present disclosure include a modifying group that is attached to the amine moiety of an amino-saccharide, e.g., mannosamine, glucosamine, galactosamine, sialic acid etc. In some embodiments, a peptide conjugate is provided comprising an intact glycosyl linking group having a formula that is selected from:

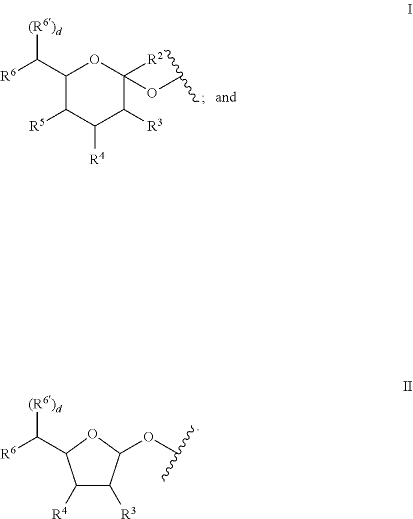

In Formulae I R² is H, CH₂OR⁷, COOR⁷ or OR⁷, in which R⁷ represents H, substituted or unsubstituted alkyl or substituted or unsubstituted heteroalkyl. When COOR⁷ is a carboxylic acid or carboxylate, both forms are represented by the designation of the single structure COO— or COOH. In Formulae I and II, the symbols R³, R⁴, R⁵, R⁶ and R^{6′} independently represent H, substituted or unsubstituted alkyl, OR⁸, NHC(O)R⁹. The index d is 0 or 1. R⁸ and R⁹ are independently selected from H, substituted or unsubstituted alkyl, substituted or unsubstituted heteroalkyl, sialic acid or polysialic acid. At least one of R³, R⁴, R⁵, R⁶ or R^{6′} includes a modifying group. This modifying group can be a polymeric modifying moiety e.g., PEG, linked through a bond or a linking group. In some embodiments, R⁶ and R^{6′}, together with the carbon to which they are attached are components of the pyruvyl side chain of sialic acid. In some embodiments, the pyruvyl side chain is functionalized with the polymeric modifying group. In some embodiments, R⁶ and R^{6′}, together with the carbon to which they are attached are components of the side chain of sialic acid and the polymeric modifying group is a component of R⁵.

Exemplary modifying group-intact glycosyl linking group cassettes according to this motif are based on a sialic acid structure, such as those having the formulae:

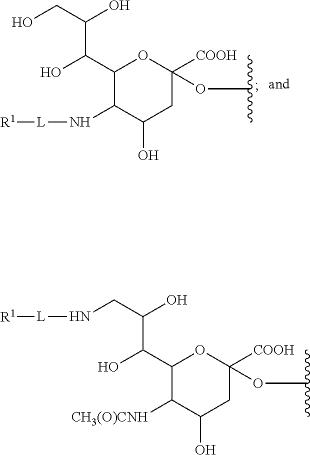

In the formulae above, R¹ and L are as described above. Further detail about the structure of exemplary R¹ groups is provided below.

In some embodiments, the conjugate is formed between a peptide and a modified sugar in which the modifying group is attached through a linker at the 6-carbon position of the modified sugar. Thus, illustrative glycosyl linking groups according to this embodiment have the formula:

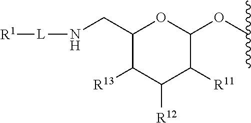

in which the radicals are as discussed above. Glycosyl linking groups include, without limitation, glucose, glucosamine, N-acetyl-glucosamine, galactose, galactosamine, N-acetylgalactosamine, mannose, mannosamine, N-acetyl-mannosamine, and the like. In some embodiments, a mutant FGF-21 peptide conjugate is provided comprising the following glycosyl linking group:

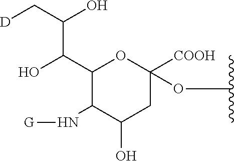

wherein D is a member selected from —OH and R¹-L-HN—; G is a member selected from H and R¹-L- and —C(O)(C₁-C₆)alkyl; R¹ is a moiety comprising a straight-chain or branched poly(ethylene glycol) residue; and L is a linker, e.g., a bond (“zero order”), substituted or unsubstituted alkyl and substituted or unsubstituted heteroalkyl. In some embodiments, when D is OH, G is and when G is —C(O)(C₁-C₆)alkyl, D is R¹-L-NH—

Aspects of the present disclosure provide a peptide conjugate that includes a glycosyl linking group having the formula:

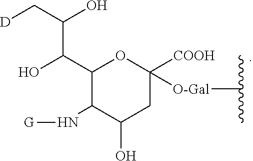

In other embodiments, the glycosyl linking group has the formula:

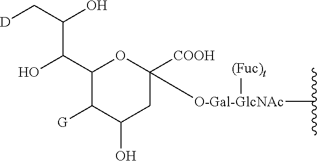

in which the index t is 0 or 1.

In some embodiments, the glycosyl linking group has the formula:

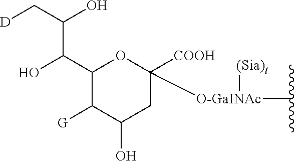

in which the index t is 0 or 1.

In yet another embodiment, the glycosyl linking group has the formula:

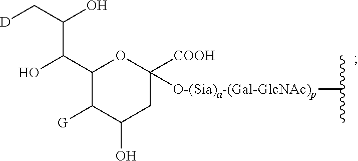

in which the index p represents and integer from 1 to 10; and a is either 0 or 1.

In some embodiments, a glycoPEGylated peptide conjugate is selected from the formulae set forth below:

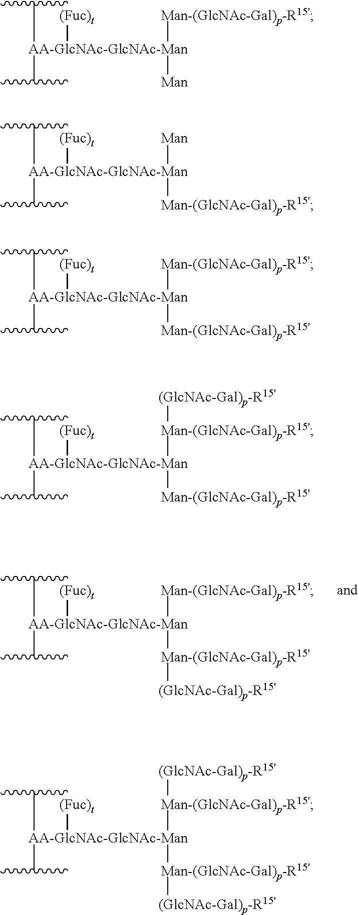

In the formulae above, the index t is an integer from 0 to 1 and the index p is an integer from 1 to 10. The symbol R^{15′} represents H, OH (e.g., Gal-OH), a sialyl moiety, a sialyl linking group (i.e., sialyl linking group-polymeric modifying group (Sia-L-R¹), or a sialyl moiety to which is bound a polymer modified sialyl moiety (e.g., Sia-Sia-L-R¹) (“Sia-Sia^{P}”)). Exemplary polymer modified saccharyl moieties have a structure according to Formulae I and II. An exemplary peptide conjugate of the present disclosure will include at least one glycan having a R^{15′} that includes a structure according to Formulae I or II.

The oxygen, with the open valence, of Formulae I and II is preferably attached through a glycosidic linkage to a carbon of a Gal or GalNAc moiety. In some embodiments, the oxygen is attached to the carbon at position 3 of a galactose residue. In some embodiments, the modified sialic acid is linked α2,3-to the galactose residue. In some embodiments, the sialic acid is linked α2,6-to the galactose residue.

In some embodiments, the sialyl linking group is a sialyl moiety to which is bound a polymer modified sialyl moiety (e.g., Sia-Sia-L-R¹) (“Sia-Sia^{P}”). Here, the glycosyl linking group is linked to a galactosyl moiety through a sialyl moiety:

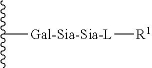

An exemplary species according to this motif is prepared by conjugating Sia-L-R¹ to a terminal sialic acid of a glycan using an enzyme that forms Sia-Sia bonds, e.g., CST-11, ST8Sia-II, ST8Sia-III and ST8Sia-IV.

In some embodiments, the glycans on the peptide conjugates have a formula that is selected from the group:

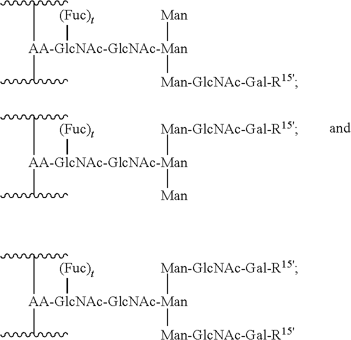

and combinations thereof.

In each of the formulae above, R^{15′} is as discussed above. Moreover, an exemplary mutant FGF-21 peptide conjugate described herein will include at least one glycan with an R¹⁵ moiety having a structure according to Formulae I or II.

In another embodiment, the glycosyl linking group comprises at least one glycosyl linking group having the formula:

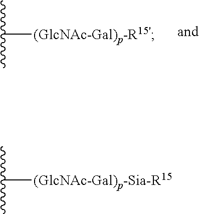

wherein R¹⁵ is said sialyl linking group; and the index p is an integer selected from 1 to 10.

In some embodiments, the glycosyl linking moiety has the formula:

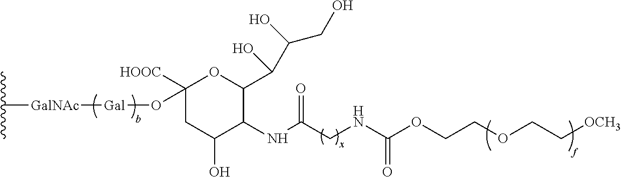

in which b is an integer from 0 to 1. The index s represents an integer from 1 to 10; and the index f represents an integer from 1 to 2500.

In some embodiments, the polymeric modifying group is PEG. In some embodiments, the PEG moiety has a molecular weight of 20-30 kDa. In some embodiments, the PEG moiety has a molecular weight of 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, or 33 kDa. In one embodiment, the PEG moiety has a molecular weight of 20 kDa. In another embodiment, the PEG moiety has a molecular weight of 30 kDa. In another embodiment, the PEG moiety has a molecular weight of about 5 kDa. In another embodiment, the PEG moiety has a molecular weight of about 10 kDa. In another embodiment, the PEG moiety has a molecular weight of about 40 kDa.

In an embodiment, the glycosyl linking group is a linear 10 kDa-PEG-sialyl, and one or two of these glycosyl linking groups are covalently attached to the peptide.

In an embodiment, the glycosyl linking group is a linear 20 kDa-PEG-sialyl, and one or two of these glycosyl linking groups are covalently attached to the peptide. In an embodiment, the glycosyl linking group is a linear 30 kDa-PEG-sialyl, and one or two of these glycosyl linking groups are covalently attached to the peptide. In an embodiment, the glycosyl linking group is a linear 5 kDa-PEG-sialyl, and one, two or three of these glycosyl linking groups are covalently attached to the peptide. In an embodiment, the glycosyl linking group is a linear 40 kDa-PEG-sialyl, and one or two of these glycosyl linking groups are covalently attached to the peptide.

In a still further embodiment, a mutant FGF-21 peptide is pegylated in accordance with methods described herein. In a particular embodiment, the mutant FGF-21 peptide comprises the mutations S¹⁷²T and R¹⁷⁶A, wherein the positions of the amino acids S and R are based on the amino acid sequence as depicted in SEQ ID NO: 1. More particularly, the mutant FGF-21 peptide comprises the amino acid sequence as depicted in SEQ ID NO: 2. As detailed herein above, the at least one glycosyl moiety attached to the threonine residue and linking the newly introduced threonine residue to the PEG moiety may virtually be any possible glycosyl moiety. The only limitation is that it should be able to attach to threonine and that it should be able to be attached to PEG or m-PEG, more particularly via a linker, e.g. an amino acid residue, particularly glycine. In particular embodiment, the at least one glycosyl moiety comprises N-acetylgalactosamine (GalNAc), galactose (Gal) and/or sialic acid (Sia). In a more particular embodiment, the at least one glycosyl moiety comprises the structure -GalNAc-Sia-, i.e. two glycosyl moieties, namely GalNAc and Sia, wherein the PEG residue may be attached to GalNAc or Sia, particularly to Sia. The glycosyl moiety which is not attached to the PEG moiety may be attached to the newly introduced threonine residue.

In another particular embodiment, the 20 kDa PEG moiety is attached to the at least one glycosyl linker via a linker, e.g. an amino acid residue, particularly a small amino acid, such as alanine or glycine, more particularly via glycine (Gly). Hence, the PEG or m-PEG moiety is attached to the amino acid and the amino acid is attached to a glycosyl moiety, such as Sia. The glycosyl moiety is attached to the amino acid linker, if present, and to the newly introduced threonine residue in the mutant FGF-21 amino acid sequence. The amino acid residue is attached to PEG and the glycosyl residue via a method described in WO 03/031464 which is incorporated herein by reference.

In a particular embodiment, the mutant FGF-21 peptide (e.g., SEQ ID NO: 2) conjugate comprises the structure -GalNAc-Sia-Gly-PEG(20 kDa), wherein GalNAc is attached, e.g. to a newly introduced threonine residue and to Sia. Sia is further attached via a glycine residue to a PEG of 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, or 33 kDa.

In a more particular embodiment, the mutant FGF-21 peptide (e.g., SEQ ID NO: 2) conjugate comprises the structure -GalNAc-Sia-Gly-PEG(20 kDa), wherein GalNAc is attached, e.g. to a newly introduced threonine residue and to Sia. Sia is further attached via a glycine residue to a PEG of 20 kDa, 21 kDa, 22 kDa, 23 kDa, 24 kDa, 25 kDa, 26 kDa, 27 kDa, 28 kDa, 29 kDa, or 30 kDa.

In a still more particular embodiment, the mutant FGF-21 peptide (e.g., SEQ ID NO: 2) conjugate comprises the structure -GalNAc-Sia-Gly-PEG(20 kDa), wherein GalNAc is attached, e.g. to a newly introduced threonine residue and to Sia. Sia is further attached via a glycine residue to a PEG of 20 kDa, 25 kDa, or 30 kDa.

In a further particular embodiment, the mutant FGF-21 peptide (e.g., SEQ ID NO: 2) conjugate comprises the structure -GalNAc-Sia-Gly-PEG(20 kDa), wherein GalNAc is attached, e.g. to a newly introduced threonine residue and to Sia. Sia is further attached via a glycine residue to a PEG of 20 kDa or 30 kDa.

In a still further particular embodiment, the mutant FGF-21 peptide (e.g., SEQ ID NO: 2) conjugate comprises the structure -GalNAc-Sia-Gly-PEG(20 kDa), wherein GalNAc is attached, e.g. to a newly introduced threonine residue and to Sia. Sia is further attached via a glycine residue to a PEG of 20 kDa.

In a very particular embodiment, the mutant FGF-21 peptide conjugate comprises the structure:

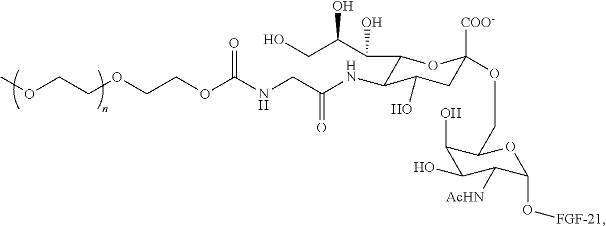

wherein n is an integer selected from 450 to 460.

The 20 kDa PEG may be linear or branched, more particularly the 20 kDa PEG, is a linear 20 kDa PEG. Further, the 20 kDa PEG is particularly a 20 kDa methoxy-PEG (mPEG, m-PEG). PEG and mPEG of different molecular weight can be obtained from various suppliers, such as from JenKem Technology USA, Plano, Tex., USA, or Merckle Biotec, Ulm, Germany. It is understood in the art that PEG 20 kDa means that the size of the PEG residues is 20 kDa in average and that the majority of the PEG residues are 20 kDa in size.

### Mutant FGF-21 Peptides and Conjugates Thereof

As described herein, the present inventors have made variants of Fibroblast Growth Factor-21 (FGF-21) having surprising properties, including variants having exceptionally long half-lives, which variants are peptide conjugates comprising

i) a mutant FGF-21 peptide comprising at least one threonine (T) residue adjacent to at least one proline (P) residue on the C-terminal side of the at least one proline residue, thereby forming at least one O-linked glycosylation site which does not exist in the corresponding native FGF-21, wherein the corresponding native FGF-21 has an amino acid sequence that is at least 95% identical to SEQ ID NO: 1, and

ii) a 20-30 kDa polyethylene glycol (PEG), wherein said 20-30 kDa PEG is covalently attached to said mutant FGF-21 peptide at the at least one threonine residue via at least one glycosyl moiety.

For the attachment of the 20-30 kDa PEG residue, a threonine residue is introduced into the amino acid sequence of native FGF-21 adjacent to and on the C-terminal side of a proline residue which is already present in the amino acid sequence of native FGF-21, i.e. is a native proline residue. For this purpose, either (i) an additional threonine may be introduced immediately next to the native proline residue or (ii) the native amino acid which is present in the native amino acid sequence of FGF-21 adjacent to and located on the C-terminal side of a native proline residue is exchanged for a threonine residue. In the present disclosure, option (ii) is an embodiment. As described herein, more than one threonine residue may be introduced adjacent and C-terminal to a proline residue which is already present. A mutant FGF-21 according to some embodiments may thus comprise both threonine residues which have been additionally introduced and threonine residues which have been introduced instead of a native amino acid.

By the introduction of a new threonine residue on the C-terminal side and adjacent to a proline residue, a consensus sequence for O-glycosylation enzyme is formed. Because proline residues are typically found on the surface of proteins (in, e.g., turns, kinks, and/or loops), a design that calls for O-glycosylation and PEGylation thereto using a PEG-glycosyl moiety in close proximity to a proline residue benefits from the relative accessibility of the target attachment site for the glycosyl transferase that transfers the glycosyl or glycol-PEG moiety and the potential to accommodate the conjugated glycosyl and/or PEG structure without disruption of protein structure.

For introduction of the threonine residues into the native amino acid sequence of FGF-21, routine techniques in the field of recombinant genetics are used. Basic texts disclosing the general methods of use in the present disclosure include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

In a particular embodiment, the native FGF-21 amino acid sequence corresponds to the native amino acid sequence of human FGF-21 depicted in SEQ ID NO: 1.

In a particular embodiment, the mutant FGF-21 peptide comprises the amino acid sequence PT, i.e. a threonine residue C-terminally adjacent to a proline residue. The sequence PT is not present in the native FGF-21 amino acid sequence.

Optionally, the mutant FGF-21 peptide comprises at least one amino acid sequence selected from the group consisting of P¹⁷²T (e.g. SEQ ID NO: 2 or SEQ ID NO: 3), P¹⁵⁶T (e.g. SEQ ID NO: 4), P⁵T (e.g. SEQ ID NO: 5), P³T (e.g. SEQ ID NO: 6), P⁹T (e.g. SEQ ID NO: 7), P⁵⁰T (e.g. SEQ ID NO: 8), P⁶¹T (e.g. SEQ ID NO: 9), P⁷⁹T (e.g. SEQ ID NO: 10), P⁹¹T (e.g. SEQ ID NO: 11), P¹¹⁶T (e.g. SEQ ID NO: 12), P¹²⁰T (e.g. SEQ ID NO: 13), P¹²⁵T (e.g. SEQ ID NO: 14), P¹²⁹T (e.g. SEQ ID NO: 15), P¹³¹T (e.g. SEQ ID NO: 16), P¹³⁴T (e.g. SEQ ID NO: 17), P¹³⁹T (e.g. SEQ ID NO: 18), P¹⁴¹T (e.g. SEQ ID NO: 19), P¹⁴⁴T (e.g. SEQ ID NO: 20, P¹⁴⁵T (e.g. SEQ ID NO: 21), P¹⁴⁸T (e.g. SEQ ID NO: 22), P¹⁵⁰T (e.g. SEQ ID NO: 23), P¹⁵¹T (e.g. SEQ ID NO: 24), P¹⁵⁸T (e.g. SEQ ID NO: 25), P¹⁵⁹T (e.g. SEQ ID NO: 26), P¹⁶⁶T (e.g. SEQ ID NO: 27), P¹⁷⁸T (e.g. SEQ ID NO: 28), and combinations thereof, wherein the positions of proline and threonine are based on the native amino acid sequence of FGF-21 as depicted in SEQ ID NO: 1, particularly the mutant FGF-21 peptide comprises at least one amino acid sequence selected from the group consisting of P¹⁷²T, P¹⁵⁶T, P⁵T and combinations thereof, more particularly consisting of P¹⁷²T, P¹⁵⁶T and combinations thereof, and even more particularly the mutant FGF-21 peptide comprises the sequence motif P¹⁷²T, based on the amino acid sequence as depicted in SEQ ID NO: 1, wherein the positions of proline and threonine are based on the amino acid sequence as depicted in SEQ ID NO: 1.

In a particular embodiment, the proline residue is located between amino acid 145 and the C-terminus of the mutant FGF-21 peptide, wherein the position of amino acid 145 is based on the amino acid sequence as depicted in SEQ ID NO: 1. As demonstrated by results presented herein, the C-terminus of FGF-21 surprisingly tolerates attachment of PEG and in particular of glycosyl-PEG moieties. This was unexpected since the literature reports that the intact C-terminus is necessary for β-Klotho binding of FGF-21.

In a particular embodiment, the mutant FGF-21 peptide comprises the mutations S¹⁷²T and R¹⁷⁶A, wherein the positions of the amino acids S and R are based on the amino acid sequence as depicted in SEQ ID NO: 1, particularly the mutant FGF-21 peptide comprises the amino acid sequence as depicted in SEQ ID NO: 2. The mutation R¹⁷⁶A has been found beneficial to the protein's overall stability after introducing the O-linked glycosylation site at threonine 173. By this mutation, the relatively large arginine side chain was removed and replaced by the small side chain of alanine. It is assumed that the smaller side chain of alanine interferes less with the voluminous glycosyl-PEG moiety to be attached to thindicae mutated FGF-21 peptide.

In an alternative embodiment, the mutant FGF-21 peptide comprises the mutation Q¹⁵⁷T, wherein the position of the amino acid Q is based on the amino acid sequence as depicted in SEQ ID NO: 1, particularly the mutant FGF-21 peptide comprises the amino acid sequence as depicted in SEQ ID NO: 4, or the mutation D⁶T, wherein the position of the amino acid D is based on the amino acid sequence as depicted in SEQ ID NO: 1, particularly the mutant FGF-21 peptide comprises the amino acid sequence as depicted in SEQ ID NO: 5.

In a particular embodiment, the mutant FGF-21 peptide conjugate comprises an amino acid sequence selected from the group consisting of SEQ ID NOs: 2 to 28, more particularly an amino acid sequence selected from the group consisting of SEQ ID NOs: 2 to 5, even more particularly an amino acid sequence selected from the group consisting of SEQ ID NOs: 2 to 4, and most particularly the mutant FGF-21 peptide comprises the amino acid sequence as depicted in SEQ ID NO: 2.

Further provided is a pharmaceutical composition comprising the mutant FGF-21 peptide conjugate and a pharmaceutically acceptable carrier, such as water or a physiologically compatible buffer. The pharmaceutical composition typically comprises a therapeutically effective or pharmaceutically active amount of the mutant FGF-21 peptide conjugate as active agent.

Pharmaceutical compositions described herein are suitable for use in a variety of drug delivery systems. Suitable formulations for use in the present disclosure can be found in Remington's Pharmaceutical Sciences, Mack Publishing Company, Philadelphia, Pa., 17^{th} ed. (1985). For a brief review of methods for drug delivery, see, Langer, Science 249: 1527-1533 (1990). The pharmaceutical compositions are intended for parenteral, intranasal, topical, oral, or local administration, such as by subcutaneous injection, aerosol inhalation, or transdermal adsorption, for prophylactic and/or therapeutic treatment. Commonly, the pharmaceutical compositions are administered parenterally, e.g., subcutaneously or intravenously. Thus, aspects of the present disclosure provide compositions for parenteral administration which comprise the mutant FGF-21 peptide conjugate dissolved or suspended in an acceptable carrier, particularly an aqueous carrier, e.g., water, buffered water, saline, phosphate buffered saline (PBS) and the like.

The compositions may also contain detergents such as Tween 20 and Tween 80; stabilizers such as mannitol, sorbitol, sucrose, and trehalose; and preservatives such as EDTA and m-cresol. The compositions may contain pharmaceutically acceptable auxiliary substances as required to approximate physiological conditions, such as pH adjusting and buffering agents, tonicity adjusting agents, wetting agents, detergents and the like.

The pharmaceutical compositions described herein may be sterilized by conventional sterilization techniques, or may be sterile filtered. The resulting aqueous solutions may be packaged for use as is, or lyophilized, the lyophilized preparation being combined with a sterile aqueous carrier prior to administration. The compositions containing the FGF peptide conjugates can be administered for prophylactic and/or therapeutic treatments, in particular for promoting weight loss in a subject in need thereof, wherein the subject in need thereof does not have symptoms associated with diabetes, particularly diabetes type 2, NASH and/or metabolic syndrome. In therapeutic applications, compositions are administered to a subject to promote weight loss in a subject in need thereof, wherein the subject in need thereof does not have symptoms associated with diabetes, particularly diabetes type 2, NASH and/or metabolic syndrome, in an amount sufficient to promote weight loss in the subject. An amount adequate to accomplish this is defined as a “therapeutically effective amount” and usually depends the patient's state of health and weight. Efficacious doses range from 0.1 mg/kg to 6 mg/kg when tested in various animal models of NASH and type 2 diabetes and such doses may be reasonably applied to promoting weight loss in subjects who do not have NASH, type 2 diabetes, or metabolic syndrome.

Aspects of the present disclosure provide methods for promoting weight loss in a subject in need thereof, wherein the subject in need thereof does not have symptoms associated with diabetes, particularly diabetes type 2, NASH, and/or metabolic syndrome, which methods comprise administering a therapeutically effective amount of a compound (a mutant FGF-21 peptide conjugate described herein) or a pharmaceutical composition comprising same to the subject (e.g., a mammal such as a human). Thus, one embodiment is a method for promoting weight loss in a subject in need thereof who does not suffer from diabetes (e.g., diabetes type 2), NASH, and/or metabolic syndrome. The method includes the step of administering to the mammal an amount of a compound described herein in an amount sufficient to promote weight loss or a composition comprising same, under conditions such that the subject loses weight following the administering. Depending on the subject's overall health and ability to restrict his/her eating behavior, weight loss could be observed within days, weeks, or months of initial administration of a compound or composition thereof as described herein.

Single or multiple administrations of the compositions can be carried out with dose levels and pattern being selected by the treating physician. In any event, the pharmaceutical compositions should provide a quantity of the mutant FGF-21 peptide conjugate described herein sufficient for an effective treatment of the subject in need of such treatment.

In the pharmaceutical composition, the mutant FGF-21 peptide conjugate is typically present in a concentration in the range from 0.1 mg/mL to 50 mg/mL, particularly from 1 mg/mL to 45 mg/mL, more particularly from 10 mg/mL to 40 mg/mL, most particularly in a concentration of 26±4 mg/mL. In a more particular embodiment, the concentration of the mutant FGF-21 peptide conjugate in a pharmaceutical composition is 33±7 mg/mL or even more particularly 26±4 mg/mL.

All components of the pharmaceutical composition, as well as the specific concentrations of the components are carefully selected after testing under many different conditions, varying compounds and concentrations thereof. Hence, the pharmaceutical composition disclosed herein is not an arbitrary selection of compounds and compound concentrations, but a specific and rational selection of conditions which are exemplary for an aqueous pharmaceutical composition containing the mutant FGF-21 peptide conjugate or mutant FGF-21 peptide when used as a medicament.

Further, the pharmaceutical composition particularly comprises a buffering agent, particularly a phosphate or Tris buffer, more particularly a Tris buffer, e.g. Tris(hydroxymethyl)aminomethane (THAM). Optionally, the buffering agent is present in a concentration from 1 mM to 100 mM, particularly from 2 mM to 75 mM, more particularly from 5 mM to 50 mM, even more particularly from 10 mM to 25 mM, most particularly of 16±2 mM. Tris buffer was selected since solubility of the protein was found to be better than for other buffer systems and it is suitable to keep the pH at pH 7.5. This pH seems the most optimal one for prolonged storage of the PEGylated mutant FGF-21 peptide conjugate. Moreover, probability of Tris cristallization at lower temperatures is lower than that of phosphate based buffering agents.

Under certain conditions, the mutant FGF-21 peptide conjugate may undergo precipitation if the pH is below 6.0. Accordingly, the pH of the pharmaceutical composition is typically maintained in the range from 6.0 to 8.5, particularly from 6.5 to 8.0, more particularly from 6.75 to 8.0, even more particularly from 7.0 to 8.0, and most particularly is 7.5±0.3 as lowest fragmentation in SDS-PAGE and least aggregation in SEC was observed if the pH is in the range of 7-8. This pH has also been identified to be optional with respect to viscosity. As the pH of a solution may depend on the temperature of the solution, the pH should particularly be adapted and measured at 25±2° C. The pH is adjusted with HCl. The pharmaceutical composition may further comprise a salt, particularly an inorganic salt, more particularly NaCl. Optionally, the salt is present in a concentration from 30 mM to 200 mM, particularly from 40 mM to 150 mM, more particularly from 50 mM to 100 mM, most particularly of 56±2 mM. The presence of a salt, particularly NaCl, is beneficial to reduce viscosity which is increased in PEG containing samples. For the same reason, it is also beneficial to include sorbitol and/or glycine.

The pharmaceutical composition may further comprise a tonicity modifying agent. The tonicity modifying agent may be selected from the group consisting of glycerol, amino acids, sodium chloride, proteins, sugars and sugar alcohols. In a particular embodiment, the tonicity modifying agent is a sugar, more particularly the tonicity modifying agent is sucrose. A tonicity modifying agent, in particular sucrose, was found to have an advantageous effect on the pharmaceutical composition as it reduces aggregation of the active agent, namely the mutant FGF-21 peptide (conjugate).

The tonicity modifying agent, particularly sucrose, may be present in a concentration of 50 mM to 200 mM, more particularly in a concentration of 100 mM to 175 mM, even more particularly in a concentration of 135 mM to 160 mM, and most particularly in a concentration of 150±2 mM.

Further, the pharmaceutical composition may comprise a surfactant, particularly a non-ionic surfactant. The surfactant or non-ionic surfactant particularly is a polysorbate-based non-ionic surfactant, more particularly polysorbate 20 or polysorbate 80, and even more particularly polysorbate 20. A surfactant, in particular polysorbate 20, was found to reduce sub-visible particles below 10 μm and thus seems to have a stabilizing effect on the pharmaceutical composition.

The surfactant or non-ionic surfactant, particularly polysorbate 20 or 80, more particularly polysorbate 20, is optionally present in a concentration of 0.01 mg/mL to 1 mg/mL, particularly in a concentration of 0.05 to 0.5 mg/mL and most particularly in a concentration of 0.2±0.02 mg/mL. Polysorbate 20 or 80, particularly polysorbate 20, were found to stabilize the formulation to aggregation.

In a particular embodiment, a pharmaceutical composition comprises 0.1 to 50 mg/mL, particularly 33±7 mg/mL of mutant FGF-21 peptide conjugate; 1 mM to 100 mM, particularly 20±2 mM, buffering agent, particularly a Tris buffer; 30 mM to 200 mM, particularly 70±2 mM, salt, particularly NaCl; and has a pH of 7.5±0.3 (particularly measured at 25±2° C.

A more particular pharmaceutical composition comprises 0.1 to 50 mg/mL, particularly 26±4 mg/mL of mutant FGF-21 peptide conjugate; 1 mM to 100 mM, particularly 16±2 mM, buffering agent, particularly a Tris buffer; 30 mM to 200 mM mM, particularly 56±2 mM, salt, particularly NaCl; 50 mM-200 mM tonicity modifying agent, particularly sucrose; and 0.01 to 1 mg/mL, particularly 0.2±0.02 mg/mL, surfactant or non-ionic surfactant, particularly polysorbate 20; and has a pH of 7.5±0.3 (particularly measured at 25±2° C.

Also provided herein is a pharmaceutical container comprising the mutant FGF-21 peptide conjugate of the present disclosure and as described herein or the pharmaceutical composition of the present disclosure and as described herein. In a particular embodiment, the pharmaceutical container is a syringe, vial, infusion bottle, ampoule, carpoule, a syringe equipped with a needle protection system, or a carpoule within an injection pen.

Aspects of the present disclosure further provide a method of producing the mutant FGF-21 peptide conjugate described herein, comprising the steps of:
(1) recombinantly producing the mutant FGF-21 peptide, particularly in an expression host; and
(2) enzymatically attaching to the mutant FGF-21 peptide of step (1) a PEG-glycosyl moiety, wherein the PEG is, for example, a 20 kDa PEG or a 30 kDa PEG, and wherein step (2) is particularly a cell free, in vitro process, thereby forming the mutant FGF-21 peptide conjugate.

In a particular embodiment, the method is as follows: First the mutation which introduces the threonine adjacent to and on the C-terminal side of a proline residue and optionally one or more further mutations are introduced into a nucleic acid sequence encoding for native or mutated FGF-21, such as of human FGF-21 as in SEQ ID NO: 1. The nucleic acid sequence encoding the mutated FGF-21 peptide is the introduced into an expression vector suitable for protein expression in an expression host. Methods for introducing mutations into nucleic acid sequences, such as site-directed mutagenesis, and the incorporation of the mutated nucleic acid sequence into an expression vector are well known to the skilled person (cf. e.g., “A Guide to Methods in the Biomedical Sciences” by R. B. Corley, Springer Science & Business Media, 2006).

After protein expression, and optional purification, the PEG residue is attached to the mutant FGF-21 peptide, specifically at the newly introduced threonine residue via at least one glycosyl moiety and optionally via at least one amino acid residue which is present between the PEG and the glycosyl residue.

To obtain high yield expression of a nucleic acid encoding a mutant FGF-21 of the present disclosure, one typically subclones a polynucleotide encoding the mutant Fibroblast Growth Factor into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al, supra. Bacterial expression systems for expressing the native or mutant FGF-21 are available in, e.g., Escherichia coli (E. coli), Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector. In a particular embodiment, the mutant FGF-21 peptide is recombinantly produced in E. coli cells, i.e. the expression host is E. coli.

An exemplary method of production is described in this paragraph: The mutant FGF-21 peptide is expressed in E. coli as inclusion bodies. Cells are recovered from the harvest by centrifugation, disrupted, and inclusion bodies are washed and recovered by centrifugation. Purification of the non-PEGylated mutant FGF-21 peptide begins with solubilizing the mutant FGF-21 peptide from the inclusion bodies and refolding of the peptide. The refolded mutant FGF-21 peptide is filtered and purified by two anion exchange chromatography operations, both utilizing Eshmuno Q chromatography resin and operated in bind and elute mode. If necessary, the purified mutant FGF-21 peptide may be concentrated by ultrafiltration using Pellicon 2 (5 kD MWCO) membranes. The purified mutant FGF-21 peptide is dispensed into sterile PETG bottles and may be stored at ≤70° C.

GlycoPEGylation of mutant FGF-21 peptide may be performed by two enzymatic reactions performed in series or at the same time. This step may be followed by 0.2 m filtration and two anion exchange chromatography operations, both utilizing Q Sepharose Fast Flow chromatography resin and operated in bind and elute mode. A final concentration step may be performed by ultrafiltration using Pellicon XL Biomax (10 kDa MWCO).

Two principal classes of enzymes are used in the synthesis of carbohydrates, glycosyltransferases (e.g., sialyltransferases, oligosaccharyltransferases, N-acetylglucosaminyltransferases), and glycosidases. The glycosidases are further classified as exoglycosidases (e.g., β-mannosidase, β-glucosidase), and endoglycosidases (e.g., Endo-A, Endo-M). Each of these classes of enzymes has been successfully used synthetically to prepare carbohydrates. For a general review, see, Crout et al., Curr. Opin. Chem. Biol. 2: 98-111 (1998). See also PCT Publication Nos: WO 2003/031464; WO 2005/089102; WO 2006/050247; and WO 2012/016984, the entire content of each of which is incorporated herein by reference.

In a particular embodiment, step (2) comprises a step (2a) of contacting the mutant FGF-21 peptide with a GalNAc donor and a GalNAc transferase under conditions suitable to transfer GalNAc from the GalNAc donor to the at least one threonine residue of the mutant FGF-21 peptide. Conditions for this transfer are described herein. Optionally, the GalNAc donor is UDP-GalNAc and, particularly, the GalNAc transferase is MBP-GalNAcT2.

In a particular embodiment and more particularly in combination with the embodiment of the aforementioned paragraph, step (2) further comprises, particularly in combination with step (2a), a step (2b) of contacting the product of step (2a), if present, or of step (1), with, e.g., a 20 kDa PEG-Sia donor or 30 kDa PEG-Sia donor and a sialyltransferase under conditions suitable to transfer 20 kDa PEG-Sia from the 20 kDa PEG-Sia donor or the 30 kDa PEG-Sia from the 30 kDa PEG-Sia donor to the at least one threonine residue of the mutant FGF-21 peptide, if step (2a) is not present, or to the GalNAc at the mutant FGF-21 peptide, if step (2a) is present. Optionally, the 20 kDa PEG-Sia donor is 20 kDa PEG-Sia-CMP or the 30 kDa PEG-Sia donor is 30 kDa PEG-Sia-CMP and/or the sialyltransferase is ST6GalNAc1. As already explained in general above, the term “20 kDa PEG-Sia” also includes “20 kDa PEG-linker-Sia” and “20 kDa PEG-Gly-Sia” and the term “30 kDa PEG-Sia” also includes “30 kDa PEG-linker-Sia” and “30 kDa PEG-Gly-Sia”.

In a more particular embodiment, the 20 kDa PEG-Sia donor comprises the structure

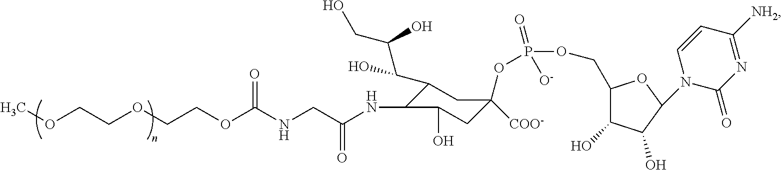

wherein n is an integer selected from 450 to 460, which results in a molecular weight of 20 kDa. This structure includes a Gly linker. The skilled person understands that methods for producing the same are described in PCT Publication No. WO 2003/031464, the entire content of which is incorporated herein by reference.

After expression and before the glycoPEGylation reaction, it is desirable to purify the mutant FGF-21 peptide. Hence, optionally, the method further comprises a step (3), after step (1) and prior to step (2), of purifying the mutant FGF-21 peptide after recombinant production. Further, the method may comprise a step (4), after step (2), of purifying the mutant FGF-21 peptide conjugate formed in step (2).

The purification step (3) and/or (4) may comprise subjecting the mutant FGF-21 peptide to a method selected from the group consisting of ion exchange chromatography, affinity chromatography, filtration and combinations thereof. Step (3) and/or step (4) may comprise one or more steps of ion exchange chromatography, affinity chromatography, filtration or combinations thereof.

Step (3) and/or step (4) may particularly comprise subjecting the mutant FGF-21 peptide to one or more steps of ion exchange chromatography, more particularly to at least two steps of ion exchange chromatography, even more particularly anion exchange chromatography. In a more particular embodiment, the mutant FGF-21 peptide is subjected to two anion exchange chromatography steps, more particularly to two strong anion exchange chromatography steps in step (3) and in step (4).

The anion exchange chromatography particularly employs a member selected from the group consisting of a hydrophilic polyvinyl ether base matrix, diethylaminoethanol (DEAE), trimethylammoniumethyl (TEAE), agarose, polystyrene/divinyl benzene polymer matrix, a quaternary ammonium (Q) strong anion exchange chromatography and combinations thereof, even more particularly in step (3) two columns using a hydrophilic polyvinyl ether base matrix are used, highly particularly in step (3) two Eshmuno®-Q columns are used. Eshmuno®-Q resins having a hydrophilic polyvinyl ether base matrix are e.g. available from Merck Millipore, Merck KGaA, Darmstadt, Germany.

Source 15Q resins are also of use in the present disclosure (GE Health Care Life Sciences, Chalfont St Giles, UK). The affinity chromatography may be an anionic anthraquinone dye affinity chromatography and filtration may employ a modified hydrophilic polyethersulfone (PES) membrane. In another embodiment, two weak anion exchange chromatography steps are performed or one strong and one weak anion exchange chromatography step.

In an alternative embodiment, the purification in step (3) is performed as below, optionally in the given order:

1. ion exchange chromatography, particularly anion exchange chromatography,

2. optionally affinity chromatography,

3. optionally ion exchange chromatography, particularly anion exchange chromatography, and

3. filtration.

Exemplary purification is performed as described in U.S. Pat. No. 9,200,049. In general, the chromatography purification steps are to be performed according to the manufacturer's protocols. Further information can e.g. be taken from “Protein Purification Protocols”, Paul Cutler, Springer Science & Business Media, 2004).

In an optional embodiment, the method further comprises a step (4), after step (2), of purifying the mutant FGF-21 peptide conjugate formed in step (2), particularly by ion exchange chromatography, more particularly by strong anion exchange chromatography, even more particularly by quaternary ammonium (Q) strong anion exchange chromatography. In a particular embodiment, two anion exchange chromatography steps are performed in step (4). Q-sepharose is a more particular column material suitable for purifying the mutant FGF-21 peptide conjugate of the present disclosure in step (4). Q sepharose is e.g. available from GE Healthcare Life Sciences, Chicago, Ill., USA.

In a particular embodiment, arginine is added in steps (2) and (3), particularly at least 400 mM arginine. Arginine is optionally added to inhibit proteases which would otherwise degrade the protein. Hence, arginine helps to prevent protein loss.

Finally, endotoxin is removed which may originate from the expression host in an optional step (5), after step (3) and prior to step (2). In this step, the product of step (3) is filtered using an endotoxin removal filter, such as Mustang E, 0.2 micron filter.

Further, the mutant FGF-21 peptide conjugate may be sterile filtered.

Also provided are the mutant FGF-21 peptide conjugates obtainable by the method of the present disclosure.

Also provided is the mutant FGF-21 peptide conjugate described herein and/or the pharmaceutical composition comprising same described herein for use as a medicament and for use in promoting weight loss in a subject in need thereof, wherein the subject does not have characteristics associated with diabetes (particularly diabetes type 2), non-alcoholic steatohepatitis (NASH), and/or metabolic syndrome. Also provided is the use of the mutant FGF-21 peptide conjugate described herein and/or the pharmaceutical composition comprising same described herein for promoting weight loss in a subject in need thereof, wherein the subject does not have characteristics associated with diabetes (particularly diabetes type 2), non-alcoholic steatohepatitis (NASH), and/or metabolic syndrome.

Further provided is a method for promoting weight loss in a subject in need thereof, wherein the subject does not have characteristics associated with diabetes (particularly diabetes type 2), non-alcoholic steatohepatitis (NASH), and/or metabolic syndrome comprising administering to the subject in need thereof an amount of the mutant FGF-21 peptide conjugate described herein and/or the pharmaceutical composition comprising same described herein. In a particular embodiment, the subject is a human subject.

In a particular embodiment, the therapeutic efficacy of a compound or composition described herein is determined based on a weight reduction in a subject after administration of the compound or composition described herein.

Also presented herein are therapeutic regimen, whereby a therapeutically effective amount of a mutant FGF-21 peptide conjugate described herein or a pharmaceutical composition comprising a therapeutically effective amount of a mutant FGF-21 peptide conjugate is administered twice per day, once per day, every two days, three times per week, once per week, once every two weeks, once every three weeks, or once per month.

Long duration efficacy of mutant FGF-21 peptide conjugates described herein is evidenced by the surprisingly long half-life determined for these conjugates in animal model systems. Long duration efficacy of mutant FGF-21 peptide conjugates described herein, in turn, makes it possible to administer the mutant FGF-21 peptide conjugates less frequently. Accordingly, in a particular embodiment, a mutant FGF-21 peptide conjugate described herein or a composition comprising same is administered to a subject in need thereof at a frequency of equal to or greater than once per week. For example, the mutant FGF-21 peptide conjugate described herein or a composition comprising same may be administered to a subject in need thereof once every 7 days, once every 8 days, once every 9 days, once every 10 days, once every 11 days, once every 12 days, once every 13 days, once every 14 days, once every 15 days, once every 16 days, once every 17 days, once every 18 days, once every 19 days, once every 20 days, once every 21 days, once every 22 days, once every 22 days, once every 23 days, once every 24 days, once every 25 days, once every 26 days, once every 27 days, once every 28 days, once every 29 days, once every 30 days, or once every 31 days.

In another exemplary therapeutic regimen, compounds described herein and compositions comprising same are following a course of “induction” therapy, which calls for more frequent administration such as twice a week or weekly at the onset of the treatment regimen followed by maintenance therapy, which may involve bi-weekly or once a month administration. Such regimens are effective in that the initial induction therapy reduces the subject's weight rapidly, thereby providing encouragement to the subject to achieve a desired maintenance weight. Thereafter, maintenance therapy is used to continue to promote weight loss, but a slower pace.

Therapeutic efficacy of a compound and/or composition for promoting weight loss in a subject in need thereof who does not have characteristics associated with diabetes (particularly diabetes type 2), non-alcoholic steatohepatitis (NASH) and/or metabolic syndrome may be evaluated using a variety of parameters and assays known by persons of skill in the art and described herein. Various methods, such as measurements using a scale to measure total body weight, displacement to determine BMI, and/or calipers to measure subcutaneous fat can be used to assess weight loss following administration of BIO89-100 or a composition thereof.

In a particular embodiment, HbA1C is measured with HPLC by using the Glycated hemoglobin test system (BIO-RAD, Hercules, Calif., USA). Blood samples (e.g., 1.0 mL/per time) may be collected from the cephalic or saphenous vein into BD Vacutainer® K2-EDTA tubes. Samples may be stored immediately at 4 degrees C. or maintained on wet ice and analyzed on the same day the blood was collected. HbA1c levels in the blood may be measured by persons skilled in the art with HPLC by using the Glycated hemoglobin test system (BIO-RAD, Hercules, Calif., USA).

With regard to NASH, this condition is currently diagnosed only by biopsy. There are some surrogate biomarkers however, that are considered predictive of NASH, such as liver fat (determined by MRI), liver enzymes (ALT and ALT/AST ratio), and fibrosis biomarkers, such as pro-C3.

### Methods

Some embodiments of the present disclosure relate to mutant Fibroblast Growth Factor-21 (FGF-21) peptide conjugates and compositions thereof described herein, as well as methods and uses for FGF-21 peptide conjugates and compositions thereof for promoting weight loss in a subject in need thereof. In a particular embodiment, the subject is not afflicted with diabetes (e.g., diabetes type 2), NASH, or metabolic syndrome. Characteristics associated with diabetes type 2, non-alcoholic steatohepatitis (NASH), and/or metabolic syndrome are known in the art and are described herein. For example, a body mass index (BMI) of 30 or greater (defined as obese) is a frequent characteristic of NASH and metabolic syndrome. Accordingly, subjects may, for example, be selected as not having NASH or metabolic syndrome based on having a BMI ranging from 25 to less than 30, or more particularly a BMI of less than 25. Subject selection based on BMI may be used alone or in combination with other selection criteria set forth herein for identifying subjects that are not afflicted with NASH or metabolic syndrome. Hemoglobin A1c (HbA1C) levels of 6.5% or higher indicate that a person has diabetes, whereas between 5.7% and 6.4% indicate that a person has a higher chance of developing diabetes. Accordingly, subjects may, for example, be selected as not having diabetes based on having an HbA1C level between 4% and 5.6%, which is considered within the normal range of HbA1C. Subject selection based on HbA1C levels may be used alone or in combination with other selection criteria set forth herein for identifying subjects that are not afflicted with diabetes. In some embodiments, the subject in which weight loss is to be promoted is a human subject.

In some embodiments, mutant Fibroblast Growth Factor-21 (FGF-21) peptide conjugates and compositions thereof described herein may be used to promote weight loss in a subject in need thereof, wherein the subject has a BMI of 30 or greater, but does not have diabetes (e.g., diabetes type 2), NASH, or metabolic syndrome.

In some embodiments, mutant Fibroblast Growth Factor-21 (FGF-21) peptide conjugates and compositions thereof described herein may be used to promote weight loss in a subject in need thereof, wherein the subject is experiencing age-related weight gain, but does not have diabetes (e.g., diabetes type 2), NASH, or metabolic syndrome.

Mutant Fibroblast Growth Factor-21 (FGF-21) peptide conjugates comprising a mutant FGF-21 peptide are described herein comprising at least one threonine residue adjacent to at least one proline (P) residue on the C-terminal side of the at least one proline residue, thereby forming at least one O-linked glycosylation site which does not exist in the corresponding native FGF-21, wherein the corresponding native FGF-21 has an amino acid sequence that is at least 95% identical to SEQ ID NO: 1, and a 20 kDa polyethylene glycol (PEG), wherein the 20 kDa PEG is covalently attached to the mutant FGF-21 peptide at the at least one threonine residue via a glycosyl moiety. In some embodiments, the mutant FGF-21 peptide conjugate comprises: a mutant FGF-21 peptide comprising an amino acid sequence of SEQ ID NO: 2, a glycosyl moiety, and a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG.

Further provided are compositions comprising same and methods for producing the mutant FGF-21 peptide conjugate and uses thereof for promoting weight loss in a subject in need thereof. In a particular embodiment, the subject is not afflicted with at least one of diabetes (e.g., diabetes type 2), NASH or metabolic syndrome. In a more particular embodiment, weight loss is promoted in a human subject. Also provided is a method for promoting weight loss in a subject in need thereof, particularly in a human subject. Also encompassed herein is the mutant FGF-21 peptide conjugate for use in promoting weight loss in a subject in need thereof, particularly in a human subject. In some embodiments, administration of the the mutant FGF-21 peptide conjugate or pharmaceutical composition comprising the mutant FGF-21 peptide conjugate results in at least one of: reduction of total body weight, reduction of body fat content, reduction of BMI of the subject or combination thereof. In some embodiments, administration of the the mutant FGF-21 peptide conjugate or pharmaceutical composition comprising the mutant FGF-21 peptide conjugate results is from 3% to 20% or more in reduction of body weight, from example, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% or more in reduction of body weight.

In another aspect, the mutant FGF-21 peptide conjugate is used in the preparation of a medicament for promoting weight loss in a subject in need thereof, particularly in a human subject.

The mutant FGF-21 peptide conjugate described herein exhibits a long half-life, which is estimated to be ˜80 hours in humans, in a subject in need thereof. Mutant FGF-21 peptide conjugates comprising a 20 kDa PEG residue also exhibit high bioavailability as reflected by 38% bioavailability in mice and rats, and 56% bioavailability in monkeys.

In an aspect, a mutant Fibroblast Growth Factor-21 (FGF-21) peptide conjugate is described herein comprising
i) a mutant FGF-21 peptide comprising the amino acid sequence of SEQ ID NO: 2,
ii) a glycosyl moiety, and
iii) a 20 kDa polyethylene glycol (PEG),
wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG. In a particular embodiment thereof, the glycosyl moiety comprises at least one of an N-acetylgalactosamine (GalNAc) residue, a galactose (Gal) residue, a sialic acid (Sia) residue, a 5-amine analogue of a Sia residue, a mannose (Man) residue, mannosamine, a glucose (Glc) residue, an N-acetylglucosamine (GlcNAc) residue, a fucose residue, a xylose residue, or a combination thereof. In another particular embodiment, the glycosyl moiety comprises at least one of an N-acetylgalactosamine (GalNAc) residue, a galactose (Gal) residue, a sialic acid (Sia), or a combination thereof. In a more particular embodiment thereof, the at least one Sia residue is a nine-carbon carboxylated sugar. Still more particularly, the at least one Sia residue is N-acetyl-neuraminic acid (2-keto-5-acetamido-3,5-dideoxy-D-glycero-D-galactononulopyranos-1-onic acid (Neu5Ac), N-glycolylneuraminic acid (Neu5Gc), 2-keto-3-deoxy-nonulosonic acid (KDN), or a 9-substituted sialic acid. In a more particular embodiment, the 9-substituted sialic acid is 9-O-lactyl-Neu5Ac, 9-O-acetyl-Neu5Ac, 9-deoxy-9-fluoro-Neu5Ac, or 9-azido-9-deoxy-Neu5Ac. In an even more particular embodiment, the glycosyl moiety comprises the structure -GalNAc-Sia-.

In an aspect, the mutant FGF-21 peptide conjugate described herein comprising the 20 kDa PEG moiety is attached to the glycosyl moiety by a covalent bond to a linker, wherein the linker comprises at least one amino acid residue. Exemplary amino acids, include: polar, but neutral amino acids (e.g., serine, threonine, cysteine, tyrosine, asparagine, and glutamine) and non-polar amino acids with relatively simple side chains (e.g. glycine, alanine, valine, leucine). In a particular embodiment, the at least one amino acid residue is at least one glycine (Gly). In a still more particular embodiment, the mutant FGF-21 peptide conjugate comprises the structure -GalNAc-Sia-Gly-PEG(20 kDa).

In an even more particular embodiment, the mutant FGF-21 peptide conjugate comprises the structure:

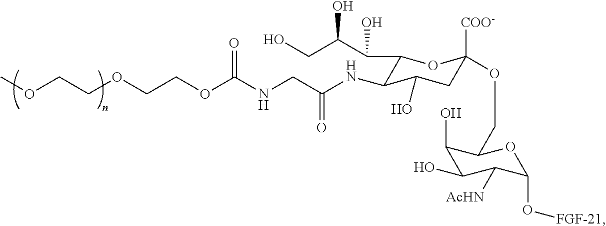

wherein n is an integer selected from 450 to 460.

A mutant FGF-21 peptide conjugate described herein may comprise a 20 kDa PEG which is a linear or branched PEG. In a more particular embodiment, the 20 kDa PEG is a linear PEG. In a still more particular embodiment, the 20 kDa PEG is a 20 kDa methoxy-PEG.

In some embodiments, the mutant FGF-21 peptide conjugate comprises:
a mutant FGF-21 peptide comprising a threonine at amino acid position 173 of SEQ ID NO: 2 to which a glycosyl moiety is attached by a covalent bond;
wherein the glycosyl comprises the structure GalNAc-Sia;
a glycine attached to the Sia;
and a linear 20 kDa PEG, wherein the 20 kDa PEG is a 20 kDa methoxy-PEG. The structure for which is presented below:

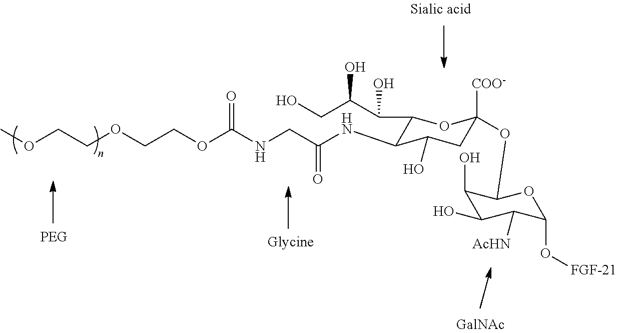

In some aspects of the disclosure, the mutant FGF-21 peptide conjugate (e.g., 89Bio-100) is administered to a human subject at a therapeutic dosing regimen of a single dose at 0.45 mg, 1.2 mg, 3 mg, 9.1 mg, 18.2 mg, 39 mg, 42 mg or 78 mg, or placebo at a 6:2 ratio (7:3 ratio for the 9.1 mg dose).

Some embodiments relate to dosage regimen whereby an effective amount of a mutant FGF-21 peptide conjugate described herein or a pharmaceutical composition comprising a therapeutically effective amount of a mutant FGF-21 peptide conjugate is administered to the subject in need thereof.

In some embodiments, the pharmaceutical composition is administered sub-subcutaneously.

In some embodiments, a therapeutically effective amount of a mutant FGF-21 peptide conjugate described herein or a pharmaceutical composition comprising a therapeutically effective amount of a mutant FGF-21 peptide conjugate is administered twice per day, once per day, every two days, three times per week, once per week, once every two weeks, once every three weeks, or once per month.

In some embodiments, a therapeutically effective amount of a mutant FGF-21 peptide conjugate described herein or a pharmaceutical composition comprising a therapeutically effective amount of a mutant FGF-21 peptide conjugate is administered once a week. In some embodiments, a therapeutically effective amount of a mutant FGF-21 peptide conjugate described herein or a pharmaceutical composition comprising a therapeutically effective amount of a mutant FGF-21 peptide conjugate is administered once every two weeks.

In some embodiments, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg of a mutant FGF-21 peptide conjugate described herein or a pharmaceutical composition comprising about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg of a mutant FGF-21 peptide conjugate is administered to the subject in need thereof twice per day, once per day, every two days, three times per week, once per week, once every two weeks, once every three weeks, or once per month.

In some embodiments, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg of a mutant FGF-21 peptide conjugate described herein or a pharmaceutical composition comprising about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg of a mutant FGF-21 peptide conjugate is administered to the subject in need thereof once a week. In some embodiments, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg of a mutant FGF-21 peptide conjugate described herein or a pharmaceutical composition comprising about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg of a mutant FGF-21 peptide conjugate is administered to the subject in need thereof once every two weeks.

In some embodiments, from about 0.08 mg/kg to about 1 mg/kg of a mutant FGF-21 peptide conjugate described herein or a pharmaceutical composition comprising from about 0.08 mg/kg to about 1 mg/kg of a mutant FGF-21 peptide conjugate is administered to the subject in need thereof twice per day, once per day, every two days, three times per week, once per week, once every two weeks, once every three weeks, or once per month. In some embodiments, from about 0.08 mg/kg to about 1 mg/kg of a mutant FGF-21 peptide conjugate described herein or a pharmaceutical composition comprising from about 0.08 mg/kg to about 1 mg/kg of a mutant FGF-21 peptide conjugate is administered to the subject in need thereof once a week. In some embodiments, from 0.08 mg/kg to about 1 mg/kg of a mutant FGF-21 peptide conjugate described herein or a pharmaceutical composition comprising from about 0.08 mg/kg to about 1 mg/kg of a mutant FGF-21 peptide conjugate is administered to the subject in need thereof once every two weeks.

In some embodiments, from about 0.1 mg/kg to about 1 mg/kg of a mutant FGF-21 peptide conjugate described herein or a pharmaceutical composition comprising from about 0.1 mg/kg to about 1 mg/kg of a mutant FGF-21 peptide conjugate is administered to the subject in need thereof twice per day, once per day, every two days, three times per week, once per week, once every two weeks, once every three weeks, or once per month. In some embodiments, from about 0.1 mg/kg to about 1 mg/kg of a mutant FGF-21 peptide conjugate described herein or a pharmaceutical composition comprising from about 0.1 mg/kg to about 1 mg/kg of a mutant FGF-21 peptide conjugate is administered to the subject in need thereof once a week. In some embodiments, from 0.1 mg/kg to about 1 mg/kg of a mutant FGF-21 peptide conjugate described herein or a pharmaceutical composition comprising from about 0.1 mg/kg to about 1 mg/kg of a mutant FGF-21 peptide conjugate is administered to the subject in need thereof once every two weeks.

In some embodiments, from about 0.1 mg/kg to about 0.2 mg/kg of a mutant FGF-21 peptide conjugate described herein or a pharmaceutical composition comprising from about 0.1 mg/kg to about 0.2 mg/kg of a mutant FGF-21 peptide conjugate is administered to the subject in need thereof twice per day, once per day, every two days, three times per week, once per week, once every two weeks, once every three weeks, or once per month. In some embodiments, from about 0.1 mg/kg to about 0.2 mg/kg of a mutant FGF-21 peptide conjugate described herein or a pharmaceutical composition comprising from about 0.1 mg/kg to about 0.2 mg/kg of a mutant FGF-21 peptide conjugate is administered to the subject in need thereof once a week. In some embodiments, from 0.1 mg/kg to about 0.2 mg/kg of a mutant FGF-21 peptide conjugate described herein or a pharmaceutical composition comprising from about 0.1 mg/kg to about 0.2 mg/kg of a mutant FGF-21 peptide conjugate is administered to the subject in need thereof once every two weeks.

In some embodiments, from about 0.1 mg/kg to about 0.3 mg/kg of a mutant FGF-21 peptide conjugate described herein or a pharmaceutical composition comprising from about 0.1 mg/kg to about 0.3 mg/kg of a mutant FGF-21 peptide conjugate is administered to the subject in need thereof twice per day, once per day, every two days, three times per week, once per week, once every two weeks, once every three weeks, or once per month. In some embodiments, from about 0.1 mg/kg to about 0.3 mg/kg of a mutant FGF-21 peptide conjugate described herein or a pharmaceutical composition comprising from about 0.1 mg/kg to about 0.3 mg/kg of a mutant FGF-21 peptide conjugate is administered to the subject in need thereof once a week. In some embodiments, from 0.1 mg/kg to about 0.3 mg/kg of a mutant FGF-21 peptide conjugate described herein or a pharmaceutical composition comprising from about 0.1 mg/kg to about 0.3 mg/kg of a mutant FGF-21 peptide conjugate is administered to the subject in need thereof once every two weeks.

In some embodiments, from about 0.1 mg/kg to about 0.4 mg/kg of a mutant FGF-21 peptide conjugate described herein or a pharmaceutical composition comprising from about 0.1 mg/kg to about 0.4 mg/kg of a mutant FGF-21 peptide conjugate is administered to the subject in need thereof. In some embodiments, from about 0.1 mg/kg to about 0.4 mg/kg of a mutant FGF-21 peptide conjugate described herein or a pharmaceutical composition comprising from about 0.1 mg/kg to about 0.4 mg/kg of a mutant FGF-21 peptide conjugate is administered to the subject in need thereof once a week. In some embodiments, from 0.1 mg/kg to about 0.4 mg/kg of a mutant FGF-21 peptide conjugate described herein or a pharmaceutical composition comprising from about 0.1 mg/kg to about 0.4 mg/kg of a mutant FGF-21 peptide conjugate is administered to the subject in need thereof once every two weeks.

In some embodiments, from about 0.1 mg/kg to about 0.5 mg/kg of a mutant FGF-21 peptide conjugate described herein or a pharmaceutical composition comprising from about 0.1 mg/kg to about 0.5 mg/kg of a mutant FGF-21 peptide conjugate is administered to the subject in need thereof twice per day, once per day, every two days, three times per week, once per week, once every two weeks, once every three weeks, or once per month. In some embodiments, from about 0.1 mg/kg to about 0.5 mg/kg of a mutant FGF-21 peptide conjugate described herein or a pharmaceutical composition comprising from about 0.1 mg/kg to about 0.5 mg/kg of a mutant FGF-21 peptide conjugate is administered to the subject in need thereof once a week. In some embodiments, from 0.1 mg/kg to about 0.5 mg/kg of a mutant FGF-21 peptide conjugate described herein or a pharmaceutical composition comprising from about 0.1 mg/kg to about 0.5 mg/kg of a mutant FGF-21 peptide conjugate is administered to the subject in need thereof once every two weeks.

In some embodiments, from about 0.3 mg/kg to about 1 mg/kg of a mutant FGF-21 peptide conjugate described herein or a pharmaceutical composition comprising from about 0.3 mg/kg to about 1 mg/kg of a mutant FGF-21 peptide conjugate is administered to the subject in need thereof twice per day, once per day, every two days, three times per week, once per week, once every two weeks, once every three weeks, or once per month. In some embodiments, from about 0.3 mg/kg to about 1 mg/kg of a mutant FGF-21 peptide conjugate described herein or a pharmaceutical composition comprising from about 0.3 mg/kg to about 1 mg/kg of a mutant FGF-21 peptide conjugate is administered to the subject in need thereof once a week. In some embodiments, from 0.3 mg/kg to about 1 mg/kg of a mutant FGF-21 peptide conjugate described herein or a pharmaceutical composition comprising from about 0.3 mg/kg to about 1 mg/kg of a mutant FGF-21 peptide conjugate is administered to the subject in need thereof once every two weeks.

In some embodiments, from about 0.5 mg/kg to about 1 mg/kg of a mutant FGF-21 peptide conjugate described herein or a pharmaceutical composition comprising from about 0.5 mg/kg to about 1 mg/kg of a mutant FGF-21 peptide conjugate is administered to the subject in need thereof twice per day, once per day, every two days, three times per week, once per week, once every two weeks, once every three weeks, or once per month. In some embodiments, from about 0.5 mg/kg to about 1 mg/kg of a mutant FGF-21 peptide conjugate described herein or a pharmaceutical composition comprising from about 0.5 mg/kg to about 1 mg/kg of a mutant FGF-21 peptide conjugate is administered to the subject in need thereof once a week. In some embodiments, from 0.5 mg/kg to about 1 mg/kg of a mutant FGF-21 peptide conjugate described herein or a pharmaceutical composition comprising from about 0.5 mg/kg to about 1 mg/kg of a mutant FGF-21 peptide conjugate is administered to the subject in need thereof once every two weeks.

In some embodiments, from about 0.08 mg/kg to about 0.8 mg/kg of a mutant FGF-21 peptide conjugate described herein or a pharmaceutical composition comprising from about 0.08 mg/kg to about 0.5 mg/kg of a mutant FGF-21 peptide conjugate is administered to the subject in need thereof twice per day, once per day, every two days, three times per week, once per week, once every two weeks, once every three weeks, or once per month. In some embodiments, from about 0.08 mg/kg to about 0.8 mg/kg of a mutant FGF-21 peptide conjugate described herein or a pharmaceutical composition comprising from about 0.08 mg/kg to about 0.8 mg/kg of a mutant FGF-21 peptide conjugate is administered to the subject in need thereof once a week. In some embodiments, from 0.08 mg/kg to about 0.8 mg/kg of a mutant FGF-21 peptide conjugate described herein or a pharmaceutical composition comprising from about 0.08 mg/kg to about 0.8 mg/kg of a mutant FGF-21 peptide conjugate is administered to the subject in need thereof once every two weeks.

In some embodiments, from about 0.08 mg/kg to about 0.2 mg/kg of a mutant FGF-21 peptide conjugate described herein or a pharmaceutical composition comprising from about 0.08 mg/kg to about 0.2 mg/kg of a mutant FGF-21 peptide conjugate is administered to the subject in need thereof twice per day, once per day, every two days, three times per week, once per week, once every two weeks, once every three weeks, or once per month. In some embodiments, from about 0.08 mg/kg to about 0.2 mg/kg of a mutant FGF-21 peptide conjugate described herein or a pharmaceutical composition comprising from about 0.08 mg/kg to about 0.2 mg/kg of a mutant FGF-21 peptide conjugate is administered to the subject in need thereof once a week. In some embodiments, from 0.08 mg/kg to about 0.2 mg/kg of a mutant FGF-21 peptide conjugate described herein or a pharmaceutical composition comprising from about 0.08 mg/kg to about 0.2 mg/kg of a mutant FGF-21 peptide conjugate is administered to the subject in need thereof once every two weeks.

In some embodiments, from about 0.08 mg/kg to about 0.3 mg/kg of a mutant FGF-21 peptide conjugate described herein or a pharmaceutical composition comprising from about 0.08 mg/kg to about 0.3 mg/kg of a mutant FGF-21 peptide conjugate is administered to the subject in need thereof twice per day, once per day, every two days, three times per week, once per week, once every two weeks, once every three weeks, or once per month. In some embodiments, from about 0.08 mg/kg to about 0.3 mg/kg of a mutant FGF-21 peptide conjugate described herein or a pharmaceutical composition comprising from about 0.08 mg/kg to about 0.3 mg/kg of a mutant FGF-21 peptide conjugate is administered to the subject in need thereof once a week. In some embodiments, from 0.08 mg/kg to about 0.3 mg/kg of a mutant FGF-21 peptide conjugate described herein or a pharmaceutical composition comprising from about 0.08 mg/kg to about 0.3 mg/kg of a mutant FGF-21 peptide conjugate is administered to the subject in need thereof once every two weeks.

In some embodiments, from about 0.08 mg/kg to about 0.4 mg/kg of a mutant FGF-21 peptide conjugate described herein or a pharmaceutical composition comprising from about 0.08 mg/kg to about 0.4 mg/kg of a mutant FGF-21 peptide conjugate is administered to the subject in need thereof twice per day, once per day, every two days, three times per week, once per week, once every two weeks, once every three weeks, or once per month. In some embodiments, from about 0.08 mg/kg to about 0.4 mg/kg of a mutant FGF-21 peptide conjugate described herein or a pharmaceutical composition comprising from about 0.08 mg/kg to about 0.4 mg/kg of a mutant FGF-21 peptide conjugate is administered to the subject in need thereof once a week. In some embodiments, from 0.08 mg/kg to about 0.4 mg/kg of a mutant FGF-21 peptide conjugate described herein or a pharmaceutical composition comprising from about 0.08 mg/kg to about 0.4 mg/kg of a mutant FGF-21 peptide conjugate is administered to the subject in need thereof once every two weeks.

In some embodiments, from about 0.08 mg/kg to about 0.5 mg/kg of a mutant FGF-21 peptide conjugate described herein or a pharmaceutical composition comprising from about 0.08 mg/kg to about 0.5 mg/kg of a mutant FGF-21 peptide conjugate is administered to the subject in need thereof twice per day, once per day, every two days, three times per week, once per week, once every two weeks, once every three weeks, or once per month. In some embodiments, from about 0.08 mg/kg to about 0.5 mg/kg of a mutant FGF-21 peptide conjugate described herein or a pharmaceutical composition comprising from about 0.08 mg/kg to about 0.5 mg/kg of a mutant FGF-21 peptide conjugate is administered to the subject in need thereof once a week. In some embodiments, from 0.08 mg/kg to about 0.5 mg/kg of a mutant FGF-21 peptide conjugate described herein or a pharmaceutical composition comprising from about 0.08 mg/kg to about 0.5 mg/kg of a mutant FGF-21 peptide conjugate is administered to the subject in need thereof once every two weeks.

In some embodiments, from about 0.08 mg/kg to about 0.4 mg/kg of a mutant FGF-21 peptide conjugate described herein or a pharmaceutical composition comprising from about 0.08 mg/kg to about 0.4 mg/kg of a mutant FGF-21 peptide conjugate is administered to the subject in need thereof twice per day, once per day, every two days, three times per week, once per week, once every two weeks, once every three weeks, or once per month. In some embodiments, from about 0.08 mg/kg to about 0.4 mg/kg of a mutant FGF-21 peptide conjugate described herein or a pharmaceutical composition comprising from about 0.08 mg/kg to about 0.4 mg/kg of a mutant FGF-21 peptide conjugate is administered to the subject in need thereof once a week. In some embodiments, from 0.08 mg/kg to about 0.4 mg/kg of a mutant FGF-21 peptide conjugate described herein or a pharmaceutical composition comprising from about 0.08 mg/kg to about 0.4 mg/kg of a mutant FGF-21 peptide conjugate is administered to the subject in need thereof once every two weeks.

In some embodiments, from about 0.1 mg to about 78 mg of a mutant FGF-21 peptide conjugate described herein or a pharmaceutical composition comprising from about 0.1 mg to about 78 mg of a mutant FGF-21 peptide conjugate is administered to the subject in need thereof once a week. In some embodiments, from about 0.1 mg to about 78 mg of a mutant FGF-21 peptide conjugate described herein or a pharmaceutical composition comprising from about 0.1 mg to about 78 mg of a mutant FGF-21 peptide conjugate is administered to the subject in need thereof once every two weeks.

In some embodiments, the effective amount of mutant FGF-21 peptide conjugate can be in a range of from about 0.1 mg to about 78 mg, a range of from about 1 mg to about 78 mg, a range of from about 3 mg to about 78 mg; a range of from about 9 mg to about 78 mg; a range of from about 18 mg to about 78 mg; a range of from about 30 mg to about 78 mg; a range of from about 40 mg to about 78 mg; a range of from about 0.1 mg to about 42 mg, a range of from about 1 mg to about 42 mg, a range of from about 3 mg to about 42 mg; a range of from about 9 mg to about 42 mg; a range of from about 18 mg to about 42 mg; a range of from about 30 mg to about 42 mg; a range of from about 3 mg to about 9 mg; a range of about 9 mg to about 18 mg; a range of about 18 mg to about 27 mg; a range of 3 mg to 18 mg and is administered once a week. For example, the effective amount of mutant FGF-21 peptide conjugate can be about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg.

In some embodiments, from about 3 mg to about 27 mg of a mutant FGF-21 peptide conjugate described herein or a pharmaceutical composition comprising from about 3 mg to about 27 mg of a mutant FGF-21 peptide conjugate is administered to the subject in need thereof once a week. In some embodiments, the effective amount of mutant FGF-21 peptide conjugate can be in a range of about 3 mg to about 27 mg; a range of about 9 mg to about 27 mg; a range of about 18 mg to about 27 mg; a range of about 3 mg to about 9 mg; a range of about 9 mg to about 18 mg; a range of about 18 mg to about 27 mg; a range of 3 mg to 18 mg and is administered once a week. For example, the effective amount of mutant FGF-21 peptide conjugate can be about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, 2 about 6 mg, or about 27 mg.

In some embodiments, from about 18 mg to about 42 mg of a mutant FGF-21 peptide conjugate described herein or a pharmaceutical composition comprising from about 18 mg to about 42 mg of a mutant FGF-21 peptide conjugate is administered to the subject in need thereof once every two weeks. For example, the effective amount of mutant FGF-21 peptide conjugate can be about 18 mg, about 19 mg, about 20 mg, about 21 mg, about 22 mg, about 23 mg, about 24 mg, about 25 mg, 2 about 6 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg.

In some embodiments, from about 18 mg to about 36 mg of a mutant FGF-21 peptide conjugate described herein or a pharmaceutical composition comprising from about 18 mg to about 36 mg of a mutant FGF-21 peptide conjugate is administered to the subject in need thereof once every two weeks. For example, the effective amount of mutant FGF-21 peptide conjugate can be about 18 mg, about 19 mg, about 20 mg, about 21 mg, about 22 mg, about 23 mg, about 24 mg, about 25 mg, 2 about 6 mg, about 27 mg, about 28 mg, about 29 mg, about 30 mg, about 31 mg, about 32 mg, about 33 mg, about 34 mg, about 35 mg, or about 36 mg.

In some embodiments, the therapeutic dosing regimen comprises a range of 3 mg to 27 mg; a range of 9 mg to 27 mg; a range of 18 mg to 27 mg; a range 3 mg to 9 mg; a range of 9 mg to 18 mg; a range of 18 mg to 27 mg; a range of 18 mg to 27 mg; a range of 3 mg to 18 mg; a range of 18 mg to 36 mg. In some embodiments, the therapeutic dosing regimen comprises a range of 3 mg to 50 mg; a range of 5 mg to 50 mg; a range of 10 mg to 50 mg; a range of 20 mg to 50 mg; a range of 30 mg to 50 mg; or a range of 40 mg to 50 mg; and any whole integer within any of the indicated ranges. In some embodiments, the therapeutic dosing regimen comprises a range of 5 mg to 40 mg; a range of 10 mg to 40 mg; a range of 20 mg to 40 mg; a range of 30 mg to 40 mg; or a range of 35 mg to 40 mg; and any whole integer within any of the indicated ranges. In some embodiments, the therapeutic dosing regimen comprises a range of 5 mg to 30 mg; a range of 10 mg to 30 mg; a range of 20 mg to 30 mg; or a range of 25 mg to 30 mg; and any whole integer within any of the indicated ranges. In some embodiments, the therapeutic dosing regimen comprises a range of 10 mg to 20 mg; or a range of 15 mg to 20 mg; and any whole integer within any of the indicated ranges. In some embodiments, the therapeutic dosing regimen comprises a dose of about 3 mg; about 9 mg; about 18 mg; or about 36 mg. The term “about” as used herein refers to an amount equal to 10% more or 10% less of the particularly indicated amount. For example, about 10 mg refers to a range of 9.0-11 mg. In yet another particular embodiment thereof, the therapeutic dosing regimen comprises a dose of 9.1 mg; about 18.2 mg; or about 39 mg.

In some embodiments, the therapeutic dosing regimen comprises a range of 0.1 mg to 78 mg; of 0.2 mg to 78 mg; of 0.4 mg to 78 mg; of 0.5 mg to 78 mg; of 0.6 mg to 78 mg; of 0.7 mg to 78 mg; of 0.8 mg to 78 mg; of 0.9 mg to 78 mg; a range of 1 mg to 78 mg; a range of 2 mg to 78 mg; a range of 3 mg to 78 mg; a range of 5 mg to 78 mg; a range of 10 mg to 78 mg; a range of 20 mg to 78 mg; a range of 30 mg to 78 mg; a range of 40 mg to 78 mg; a range of 50 mg to 78 mg, a range of 60 mg to 78 mg or a range of 70 mg to 78 mg and any whole integer within any of the indicated ranges.

In some embodiments, the therapeutic dosing regimen comprises a range of 0.1 mg to 50 mg; of 0.2 mg to 50 mg; of 0.4 mg to 50 mg; of 0.5 mg to 50 mg; of 0.6 mg to 50 mg; of 0.7 mg to 50 mg; of 0.8 mg to 50 mg; of 0.9 mg to 50 mg; a range of 1 mg to 50 mg; a range of 2 mg to 50 mg; a range of 3 mg to 50 mg; a range of 5 mg to 50 mg; a range of 10 mg to 50 mg; a range of 20 mg to 50 mg; a range of 30 mg to 50 mg; or a range of 40 mg to 50 mg; and any whole integer within any of the indicated ranges.

In some embodiments, the therapeutic dosing regimen comprises a range of 0.1 mg to 40 mg; of 0.2 mg to 40 mg; of 0.4 mg to 40 mg; of 0.5 mg to 40 mg; of 0.6 mg to 40 mg; of 0.7 mg to 40 mg; of 0.8 mg to 40 mg; of 0.9 mg to 40 mg; a range of 1 mg to 40 mg; a range of 2 mg to 40 mg; a range of 3 mg to 40 mg; a range of 4 mg to 40 mg; a range of 5 mg to 40 mg; a range of 10 mg to 40 mg; a range of 20 mg to 40 mg; a range of 30 mg to 40 mg; or a range of 35 mg to 40 mg; and any whole integer within any of the indicated ranges.

In some embodiments, the therapeutic dosing regimen comprises a range of 0.1 mg to 30 mg; of 0.2 mg to 30 mg; of 0.4 mg to 30 mg; of 0.5 mg to 30 mg; of 0.6 mg to 30 mg; of 0.7 mg to 30 mg; of 0.8 mg to 30 mg; of 0.9 mg to 30 mg; a range of 1 mg to 30 mg; a range of 2 mg to 30 mg; a range of 3 mg to 30 mg; a range of 4 mg to 30 mg; a range of 5 mg to 30 mg; a range of 10 mg to 30 mg; a range of 20 mg to 30 mg; or a range of 25 mg to 30 mg; and any whole integer within any of the indicated ranges.

In some embodiments, the therapeutic dosing regimen comprises a range of 0.1 mg to 20 mg; of 0.2 mg to 20 mg; of 0.4 mg to 20 mg; of 0.5 mg to 20 mg; of 0.6 mg to 20 mg; of 0.7 mg to 20 mg; of 0.8 mg to 20 mg; of 0.9 mg to 20 mg; a range of 1 mg to 20 mg; a range of 2 mg to 20 mg; a range of 3 mg to 20 mg; a range of 4 mg to 20 mg; a range of 5 mg to 20 mg; a range of 10 mg to 20 mg; or a range of 15 mg to 20 mg; and any whole integer within any of the indicated ranges.

In some embodiments, the therapeutic dosing regimen comprises a dose of about 0.45 mg, a dose of about 1.2 mg, a dose of about 3 mg, a dose of about 9 mg; about 18 mg; or about 39 mg.

The term “about” as used herein refers to an amount equal to 10% more or 10% less of the particularly indicated amount. For example, about 10 mg refers to a range of 9.0-11 mg. In yet another particular embodiment thereof, the therapeutic dosing regimen comprises a dose of 9.1 mg; about 18.2 mg; or about 39 mg.

In some embodiments, a therapeutically effective amount of a mutant FGF-21 peptide conjugate described herein or a pharmaceutical composition comprising a therapeutically effective amount of a mutant FGF-21 peptide conjugate is administered twice per day, once per day, every two days, three times per week, once per week, once every two weeks, once every three weeks, or once per month.

The aforementioned therapeutic dosing regimens may be administered to a human in need thereof to promote weight loss in a subject via a variety of modes known to those skilled in the art, including without limitation: subcutaneous administration.

The aforementioned therapeutic dosing regimens may be administered to a human in need thereof to promote weight loss in a subject alone or in combination with other weight loss regimen such as those known in the art and described herein. Examples of other weight loss regimen currently used to promote weight loss include, without limitation, reduced caloric intake, increased metabolic expenditure (via, e.g., exercise), and weight loss promoting therapeutic agents.

The aforementioned therapeutic dosing regimens may be administered to a human in need thereof to promote weight loss in a subject, wherein the subject does not have diabetes (e.g., diabetes type 2), non-alcoholic steatohepatitis (NASH), or metabolic syndrome. In a particular embodiment, the aforementioned therapeutic dosing regimens are administered to a subject (e.g., a human) in need thereof to reduce fat content of the subject.

In another aspect, a pharmaceutical composition comprising any one of or at least one of the mutant FGF-21 peptide conjugates described herein and a pharmaceutically acceptable carrier is presented. The mutant FGF-21 peptide conjugate may be present in the pharmaceutical composition in a concentration in a range from 0.1 mg/mL to 50 mg/mL, particularly from 1 mg/mL to 45 mg/mL, more particularly from 10 mg/mL to 40 mg/mL, most particularly in a concentration of 26±4 mg/mL. In a particular embodiment, the pharmaceutical composition further comprises a buffering agent, particularly a Tris buffer. In another embodiment, the buffering agent is present in a concentration from 1 mM to 100 mM, particularly from 2 mM to 75 mM, more particularly from 5 mM to 50 mM, even more particularly from 10 mM to 25 mM, most particularly of 16±2 mM. More particularly, the pH is in the range from 6.0 to 8.5, particularly from 6.5 to 8.0, more particularly from 6.75 to 8.0, and most particularly is 7.5±0.3. In another particular embodiment, the pharmaceutical composition further comprises a salt, particularly an inorganic salt, more particularly NaCl. More particularly, the salt is present in a concentration from 30 mM to 200 mM, particularly from 40 mM to 150 mM, more particularly from 50 mM to 100 mM, most particularly of 56±2 mM. The pharmaceutical composition may further comprise a tonicity modifying agent. Such tonicity modifying agents include, without limitation, glycerol, amino acids, sodium chloride, proteins, sugars and sugar alcohols, particularly the tonicity modifying agent is a sugar, more particularly the tonicity modifying agent is sucrose. In another embodiment, the the tonicity modifying agent is present in a concentration of 50 mM to 200 mM, more particularly in a concentration of 100 mM to 175 mM, even more particularly is present in a concentration of 135 mM to 160 mM, and most particularly in a concentration of 150±2 mM. In another embodiment, the pharmaceutical composition further comprises a surfactant, particularly a non-ionic surfactant, wherein the surfactant or non-ionic surfactant is a polysorbate-based non-ionic surfactant, particularly polysorbate 20 or polysorbate 80, more particularly polysorbate 20. In a particular embodiment, the surfactant or non-ionic surfactant is present in a concentration of 0.01 mg/mL to 1 mg/mL, particularly in a concentration of 0.05 to 0.5 mg/mL and more particularly in a concentration of 0.2±0.02 mg/mL.

In some embodiments, the pharmaceutical composition comprises 0.1 mg/mL to 50 mg/mL of mutant FGF-21 peptide conjugate, 1 mM to 100 mM buffering agent, particularly Tris buffer, 30 mM to 200 mM mM salt, particularly NaCl, 50 mM to 200 mM tonicity modifying agent, particularly sucrose, and 0.01 mg/mL to 1 mg/mL surfactant or non-ionic surfactant, particularly polysorbate 20, and has a pH of 6.0 to 8.5. Also encompassed herein is a pharmaceutical container comprising any one of or at least one of a mutant FGF-21 peptide conjugate described herein or a pharmaceutical composition comprising same. Exemplary such pharmaceutical containers include, without limitation, a syringe, vial, infusion bottle, ampoule, carpoule, a syringe equipped with a needle protection system, or a carpoule within an injection pen.

In a further aspect, a method of producing a mutant FGF-21 peptide conjugate described herein is presented, comprising the steps of:
(1) recombinantly producing the mutant FGF-21 peptide in an expression host; and
(2) enzymatically attaching to the mutant FGF-21 peptide of step (1) a PEG-glycosyl moiety, wherein the PEG has 20 kDa, thereby forming the mutant FGF-21 peptide conjugate. In a particular embodiment, the expression host is Escherichia coli. In another particular embodiment of the method, step (2) comprises a step (2a) of contacting the mutant FGF-21 peptide with a GalNAc donor and a GalNAc transferase under conditions suitable to transfer GalNAc from the GalNAc donor to the threonine at amino acid position 173 of SEQ ID NO: 2. In a still more particular embodiment, the GalNAc donor is UDP-GalNAc. In yet another particular embodiment, the GalNAc transferase is MBP-GalNAcT2. In another particular embodiment of the method, step (2) further comprises a step (2b) of contacting the product of step (1) or of step (2a), if present, with a 20 kDa PEG-Sia donor and a sialyltransferase under conditions suitable to transfer 20 kDa PEG-Sia from the 20 kDa PEG-Sia donor to the threonine residue at amino acid position 173 of SEQ ID NO: 2 or to the GalNAc attached to the threonine residue at amino acid position 173 of SEQ ID NO: 2 if step (2a) is present. In a further particular embodiment of the method, the 20 kDa PEG-Sia donor is 20 kDa PEG-Sia-CMP. In a still more particular embodiment of the method, the sialyltransferase is ST6GalNAc1. In an even more particular embodiment of the method, the 20 kDa PEG-Sia donor comprises the structure

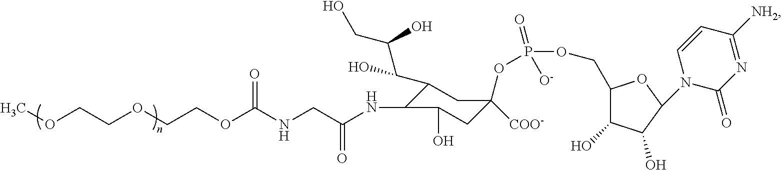

wherein n is an integer selected from 450 to 460.

In another particular embodiment of the method, the method further comprises a step (3), after step (1) and prior to step (2), of purifying the mutant FGF-21 peptide after recombinant production. The method may further comprise a step (4), after step (2), of purifying the mutant FGF-21 peptide conjugate formed in step (2). In a particular embodiment, step (3) comprises subjecting the mutant FGF-21 peptide and/or step (4) comprises subjecting the mutant FGF-21 peptide conjugate to a method selected from the group consisting of ion exchange chromatography, affinity chromatography, filtration and combinations thereof. The step of purifying may comprise one or more steps of ion exchange chromatography, particularly two steps of ion exchange chromatography. In a particular embodiment thereof, the ion exchange chromatography is an anion exchange chromatography, particularly a strong anion exchange chromatography. In a particular embodiment thereof, the anion exchange chromatography employs a member selected from the group consisting of a hydrophilic polyvinyl ether base matrix, polystyrene/divinyl benzene polymer matrix, trimethylammoniumethyl (TEAE), diethylaminoethanol (DEAE), agarose, a quaternary ammonium (Q) strong anion exchange chromatography and combinations thereof. In another particular embodiment thereof, step (3) comprises two anion exchange chromatography steps using a hydrophilic polyvinyl ether base matrix. In another particular embodiment thereof, step (4) comprises two quaternary ammonium (Q) strong anion exchange chromatography steps. In particular embodiment, arginine is added in step (2) and/or, if present, in step (3), particularly at least 400 mM arginine. In another particular embodiment, the method further comprises a step (5), after step (3) and prior to step (2), of endotoxin removal, wherein the product of step (3) is filtered using an endotoxin removal filter.

Also encompassed herein is a mutant FGF-21 peptide conjugate obtainable by any one of the methods described herein.

In another aspect, a method for promoting weight loss in a subject in need thereof who does not have at least one of diabetes type 2, non-alcoholic steatohepatitis (NASH), or metabolic syndrome is presented, comprising administering to the subject in need thereof an amount of a mutant FGF-21 peptide conjugate described herein or obtainable by a method described herein or a pharmaceutical composition comprising same. In a particular embodiment, the subject is a human subject. In a more particular embodiment, the administering reduces at least one of the total weight of the subject, body fat content of the subject, or BMI. As described herein, methods comprising administering to a subject an amount of a mutant FGF-21 peptide conjugate described herein or obtainable by a method described herein or a pharmaceutical composition comprising same may be used alone or in combination with other therapeutic regimens accepted for use promoting weight loss in a subject.

Also encompassed herein is any one of the mutant FGF-21 peptide conjugates described herein or a pharmaceutical composition comprising same for use in promoting weight loss in a subject in need thereof who does not have at least one of diabetes type 2, non-alcoholic steatohepatitis (NASH), or metabolic syndrome. In a particular embodiment, the subject is a human subject. In a more particular embodiment, the use reduces at least one of the total weight of the subject, body fat content of the subject, or BMI. As described herein, use of an amount of a mutant FGF-21 peptide conjugate described herein or obtainable by a method described herein or a pharmaceutical composition comprising same may be used alone or in combination with other active pharmaceutical agents or therapeutic interventions used for promoting weight loss in a subject.

In another aspect, use of a mutant FGF-21 peptide conjugate described herein in the preparation of a medicament for promoting weight loss in a subject in need thereof who does not have at least one of diabetes type 2, non-alcoholic steatohepatitis (NASH), or metabolic syndrome is presented. In a particular embodiment, the subject is a human subject. In a more particular embodiment, the use reduces at least one of the total weight of the subject, body fat content of the subject, or BMI.

In another aspect, a mutant Fibroblast Growth Factor-21 (FGF-21) peptide conjugate is presented comprising a mutant FGF-21 peptide comprising the amino acid sequence of SEQ ID NO: 2, a glycosyl moiety, wherein the glycosyl moiety comprises the structure -GalNAc-Sia-, and a 30 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 30 kDa PEG by a covalent bond between a second site of the glycosyl moiety and the 30 kDa PEG. In a particular embodiment, the 30 kDa PEG moiety is attached to the glycosyl moiety by a covalent bond to a linker, wherein the linker comprises at least one amino acid residue. Exemplary amino acids, include: polar, but neutral amino acids (e.g., serine, threonine, cysteine, tyrosine, asparagine, and glutamine) and non-polar amino acids with relatively simple side chains (e.g. glycine, alanine, valine, leucine). In a particular embodiment, the at least one amino acid residue is at least one glycine (Gly). In a still more particular embodiment, the mutant FGF-21 peptide conjugate comprises the structure -GalNAc-Sia-Gly-PEG(30 kDa). A mutant FGF-21 peptide conjugate described herein may comprise a 30 kDa PEG which is a linear or branched PEG. In a more particular embodiment, the 30 kDa PEG is a linear PEG. In a still more particular embodiment, the 30 kDa PEG is a 30 kDa methoxy-PEG.

All prior patents, publications, and test methods referenced herein are incorporated by reference in their entireties.

Variations, modifications and alterations to embodiments of the present disclosure described above will make themselves apparent to those skilled in the art. All such variations, modifications, alterations and the like are intended to fall within the spirit and scope of the present disclosure, limited solely by the appended claims.

While several embodiments of the present disclosure have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

Any feature or element that is positively identified in this description may also be specifically excluded as a feature or element of an embodiment of the present as defined in the claims.

### Examples

Mutant FGF21-GalNAc-SA-PEG-20 kDa (BIO89-100) reduces body weight and fat mass in naive CD-1 mice via an increase in energy expenditure despite an increase in food.

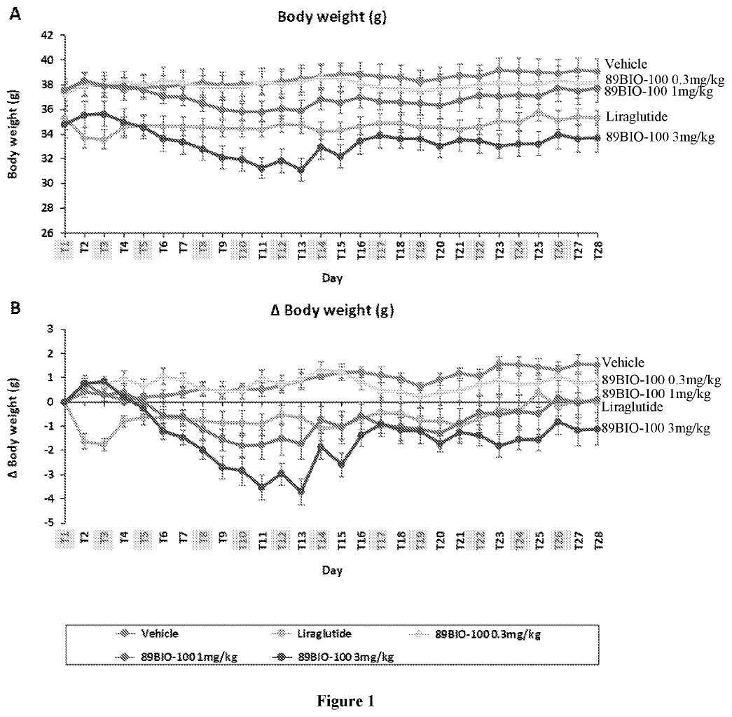

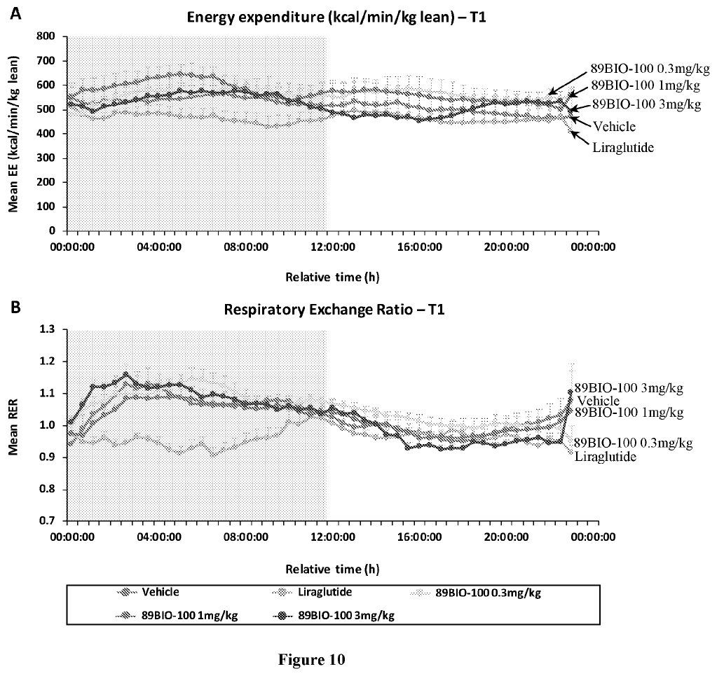

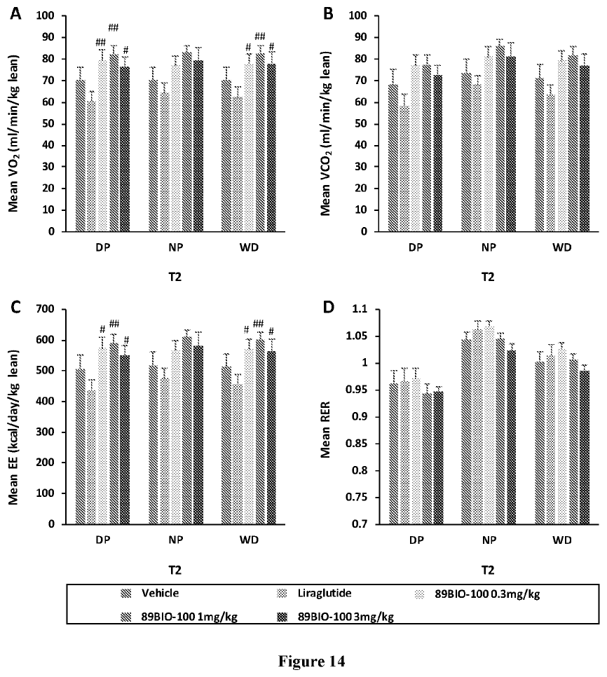

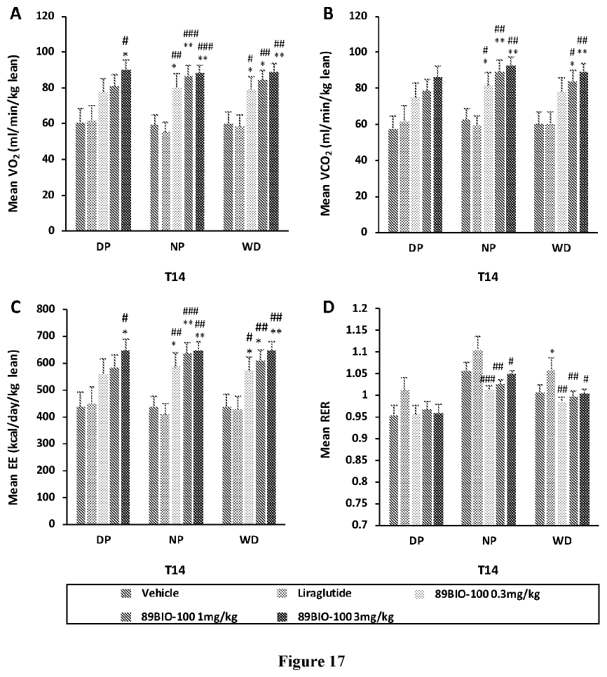

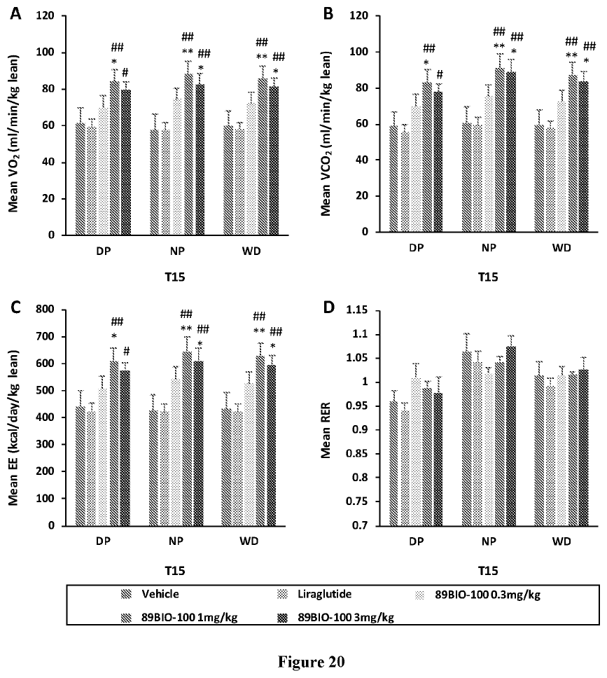

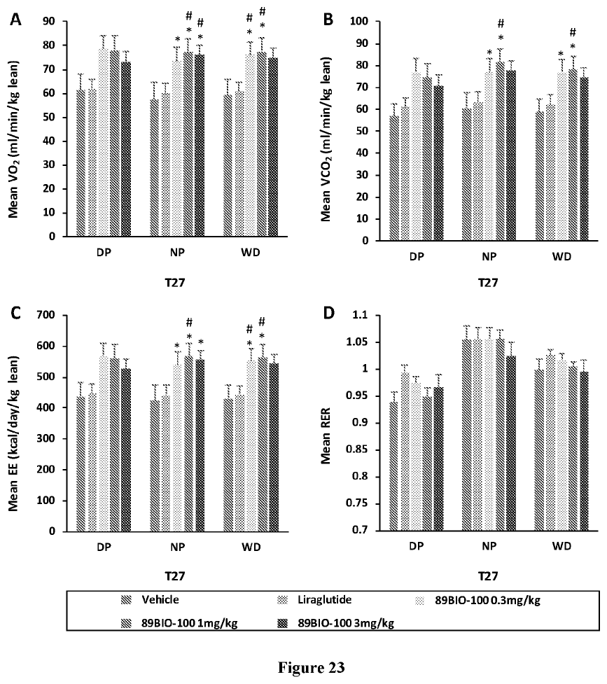

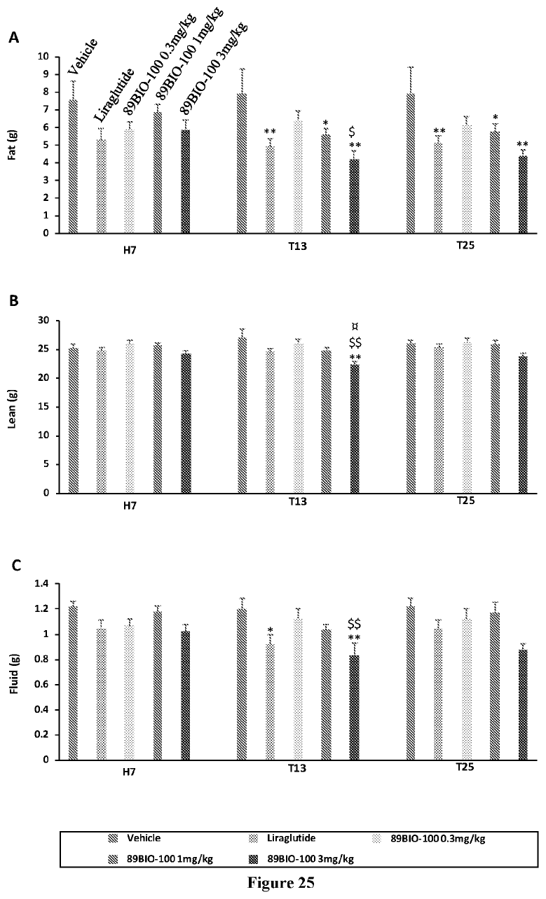

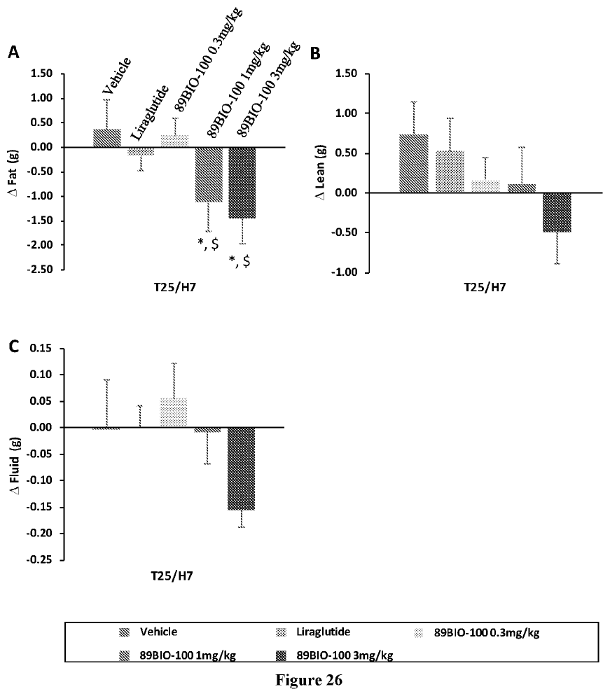

As detailed herein, the present inventors discovered that in CD-1 mice, CD-1 IGS mice are outbred mice derived from a group of outbred Swiss mice (a representative healthy mouse strain), administration of BIO89-100 reduces body weight (BW) despite increased food consumption (FC). To better understand these findings, the present inventors investigated the effect of BIO89-100 on energy expenditure and body composition. A dose-dependent decrease in BW (up to −13.8%) and increase in FC (up to +94%) were observed in BIO89-100-treated mice. See, for example, FIGS. 1A, and 2A-7F. Increased FC was related to an increase in mean meal number, with no change in mean meal size. Although the Basal Metabolic Rate (BMR) was not directly measured during the study, the energy expenditure modulations observed during the diurnal period (daytime which is the resting period for mice) suggests that there is a slight change in the basal energy expenditure. See, for example, FIGS. 10A-23D. Given the magnitude of the effects of BIO89-100 on energy expenditure, it seems unlikely that an increase in BMR alone could be the sole cause for increased energy expenditure. Thus, it was concluded that thermogenesis is increased under the effect of BIO89-100. BIO89-100-treated CD-1 mice had a marked decrease in fat mass without affecting lean masses and body fluid. See, for example, FIGS. 25A-26C. Liraglutide induced a significant decrease in BW when compared with controls resulting in a final 10% reduction, associated with a decrease in fat mass. However, liraglutide did not affect the energy expenditure or food consumption.

Accordingly, the present inventors have discovered that naive CD-1 mice treated with BIO89-100 had a dose-dependent reduction in BW and increased FC. These results indicate that BIO89-100-mediated body weight loss is due, at least in part, to an increase in energy expenditure, resulting in a marked decrease in fat mass, without affecting lean masses and body fluid. The results, therefore, demonstrate that BIO89-100 may be used to advantage to promote weight loss in normal, healthy subjects. In a particular embodiment, such subjects are selected as not being afflicted with diabetes (e.g., diabetes type 2), NASH, and/or metabolic syndrome. Accordingly, suitable subjects for treatment in accordance with methods described herein can be selected for having characteristics that fall outside of diagnostic criteria accepted with respect to diabetes (e.g., diabetes type 2), NASH, and/or metabolic syndrome.

Background: The CD-1 mouse is considered a representative healthy mouse strain. More particularly, Crl:CD1(ICR)) is an albino outbred strain of a mouse model that has frequently been used in toxicology and pharmacological research to evaluate potential toxicity of therapeutic agents. It is the most popular strain of outbred mouse. CD1 mice are albino and thus, have completely white fur. A notable feature of CD1 mice is their large genetic diversity, which is similar to that found within and between human populations.

Detailed Methods: Adult male CD-1 mice at 6 weeks of age at receipt (with an expected weight of 35-39 g) were purchased from Charles River (St Germain sur l'Arbresle, France). From receipt, mice were singly housed and allowed ad libitum access to water and to a control diet (pellet A04; SAFE, Villemoisson-sur-Orge, France), unless otherwise noted (see Section D.). Throughout the study, animals were maintained in an ambient temperature (22.0±1.0° C.) and humidity (40-50%) controlled room on a 12-h light/12-h dark cycle (9:00 PM: Light ON/9:00 AM Light OFF).

### Body Composition Analyzer

For body composition analyses, a minispec Analyzer (LF90II, Bruker, Germany) was used. The LF90II provides an accurate measure of the whole-body lean mass, fat mass and free fluid mass in living and vigil animals.

### Physiocage System (METABOpack™)

During the METABOpack™ sessions, animals were housed singly in the Physiocage System (Panlab/Harvard apparatus), which was composed of:
8 physiocages (LE1301)
8 Air tight lids for mouse (LE1303)
8 grid floors for mouse (LE1317)
8 Feeding bottles for mouse (LE1306)
2 Air supply and switching units for up to 4 home cages each (LE4004FL)
1 O2/CO2 analyzer (LE405)
1 acquisition software suite, METABOLISM (V3.0).

The Physiocage System notably allows recording the following parameters:
Food intake (1 measure/sec; g)
Water intake (1 measure/sec; ml)
Spontaneous locomotor activity (1 measure/sec; arbitrary unit)
VO₂ (1 measure/sec for 3 min, every 30 min; ml/min/kg lean)
VCO₂ (1 measure/sec for 3 min, every 30 min; ml/min/kg lean)

### Computer Systems and Software

Data acquisition during the METABOpack™ sessions was performed using the software suite METABOLISM V3.0 (Panlab/Harvard apparatus) on a computer system (Dell Vostro 3500 BTS, Windows 7 32 bits). All the other data were entered into an Excel file that was stored in a central computer server.

At receipt and until the end of the experiment, all animals fed the control diet (A04 pellet). Following the acclimation and habituation periods, forty-eight (48) mice were subcutaneously treated either with vehicle, liraglutide (as the benchmark) or 89BIO-100 (3 different doses tested).

### Vehicle

Denomination: BIO89-100 Vehicle.

Preparation: A solution stock (1 L) of Vehicle was prepared just before the first day of dosing and was used for the whole study. Briefly, 2.5 g of Tris HCl (16 mM), 3.27 g of NaCl (56 mM) and 51.35 g of sucrose (150 mM) were weighted and added to 1 L of distilled water. The solution was stirred until complete dissolution. Then, 200 μl of Polysorbate 20 (0.2 mg/ml) were added to the solution under stirring and the pH of the solution was adjusted to 7.5. Finally, the solution stock was stored at +4° C.

On each dosing day, an adequate volume of Vehicle was removed from +4° C. and allowed to warm to room temperature for at least 30 minutes before use.

Frequency of preparation: once for the whole study.

Storage: at 2-8° C. (refrigerator).

Liraglutide (benchmark)

Denomination: Liraglutide (Cayman Chemical Company, Ann Arbor, Mich., USA)

Preparation: Formulations were prepared on each dosing day by diluting the Liraglutide stock in a sodium chloride solution (0.9% NaCl) to obtain a final concentration of 0.04 mg/mL. On each dosing day and before the first dosing, one fresh aliquot of Liraglutide was removed from −20° C. and kept at room temperature for at least 30 minutes prior the administration. Before the second dosing of the day, another fresh aliquot of Liraglutide was used and prepared as mentioned above.

Frequency of preparation: twice a day of animal dosing.

Storage: −20° C. 89BIO-100 (3 doses: 0.3, 1 and 3 mg/kg) Denomination: BIO89-100.

Preparation: Formulations were prepared on each dosing day by diluting the BIO89-100 stock with the Vehicle Control at a concentration range of 0.03-0.3 mg/mL, to meet dose level requirements. The Test Item was thawed under ambient conditions (room temperature) and was divided to the required aliquots based on the number of dosing days. Briefly, the bottle was checked frequently (target: every 30 minutes) during the thawing process for ice. During the check and to optimize the thawing process, the bottle was gently swirled to mix the content without shaking aggressively or causing foaming. The thawing process was considered complete once no remaining evidence of ice was in the material. The thawing process would take up to 4 hours.

On each dosing day, once the aliquot was thawed, the material was kept on ice and maintained at 2 to 8° C. until the dilution process needed to obtain the desired concentrations. The dosing formulation preparation was performed on wet ice. The time at room temperature for the thawed Test Item was avoided, where possible, or was not exceeded 30 min. The dosing formulation after dilution was stored in a refrigerator set to maintain at 2 to 8° C., protected from light with aluminium foil. Prior to administration, the prepared dosing formulations were removed from wet ice and allowed to warm up to room temperature for at least 30 minutes before dosing and used within 4 hours after being placed at room temperature.

Frequency of preparation: every day of animal dosing.

Storage: at −20° C.

### Experimental Procedures

### Acclimation/Habituation Periods and Matching Procedure

For seven (7) days of Acclimation period from receipt (A1-A7), animals were acclimated to their new environment. Both body weight (BW) and food intake (FI) were measured on the same days, two (2) times (at A1 and A4), in order to ensure an optimal habituation to Biomeostasis' animal facilities.

On Habituation period, from habituation day 1 (H1) to habituation day 7 (H7), animals were daily habituated to subcutaneous administration. Both body weight (BW) and food intake (FI) were measured on the same days, two (2) times (at H1 and H4), in order to ensure an optimal habituation of the route administration.

On habituation day 4 (H4), mice were matched on the basis of their FI and BW values in order to form as many as homogenous experimental groups of eight (8) animals and labelled as followed in Table 3:

| | Dose | Vol- | | Dosing | Body | Food |
|---|---|---|---|---|---|---|
| | (mg/ | ume | Group | route/ | weight (g) | intake (g) |
| Groups | kg) | mL/kg | size | regimen | on H₄ | on H₄ |
| Vehicle | 0 | 10 | n = 10* | SC/3 per | 37.19 ± 0.79 | 7.79 ± 0.72 |
| | | | | week | | |
| Liraglutide | 0.2 | 5 | n = 8 | SC BID | 35.11 ± 1.15 | 9.08 ± 1.64 |
| 89BIO-100 | 0.3 | 10 | n = 11 | SC/3 per | 36.56 ± 0.82 | 9.02 ± 1.37 |
| | | | | week | | |
| 89BIO-100 | 1 | 10 | n = 11 | SC/3 per | 36.87 ± 0.55 | 12.23 ± 0.97 |
| | | | | week | | |
| 89BIO-100 | 3 | 10 | n = 8 | SC/3 per | 35.20 ± 1.13 | 8.32 ± 1.03 |
| | | | | week | | |

| | | |
|---|---|---|
| F = | 1.169 | 1.198 |
| P = | 0.3377 | 0.3254 |

### Body Composition

Body composition was measured using a minispec Analyzer (LF90II, Bruker, Germany) one (1) day before treatment start on habituation day 7 (H₇) and on treatment day 13 (T₁₃) and treatment day 25 (T₂₅) of Treatment days. Briefly, each mouse was placed in a red clear plastic tube sealed by a plunger. Then, the tube was inserted into the minispec Analyzer for measurement of body composition (˜ 2 min/measurement). Once the measurement completed, the mouse was removed from the plastic tube and immediately replaced into its cage. The tube and the plunger were washed and dried between each mouse. As a reminder, the Minispec analyser allows fast and precise measurements of fat tissue (g) (adipose tissue), lean tissue (g) (mainly muscles) and free fluid content (g). It should be noted that each measurement was performed just prior to a METABOpack™ session in order to normalize the respiratory exchanges-related parameters of each mouse by its lean mass (see METABOpack™ session section).

### Treatment Period

From T₁ to T₂₈, vehicle and 89BIO100 (at the 3 different doses) were administrated by subcutaneous (SC) injection three times a week, while the treatment with liraglutide was administrated by subcutaneous route, twice a day, every day. It was important to notice that the first dosing was done just before the dark cycle (at 9:00 AM) and the second dosing was done 8 hours after the first one (at 5:00 PM). Throughout the treatment period, FI and BW were daily measured.

Rationale for dose level selection From T₁ to T₂₈, 89BIO100 was administrated by subcutaneous route (SC) with dose levels of 0.3, 1 and 3 mg/kg. The 0.3 mg/kg (3 times/week) dose level was demonstrated to be effective in Diet-induced NASH model in mice while the 1 mg/kg (3 times/week) dose level was used in the 28-day GLP general toxicology study in CD-1 mice, allowing the investigation of body weight reduction.

Liraglutide dose of 0.2 mg/kg (SC route, bis-day) was selected because of its effectiveness in reducing body weight, food intake and increasing insulin sensitivity in obese and diabetic rodent models.

### Semi-Fasted Glycemia

On H₇, T₃, T₇ and T₂₈, blood glucose was measured from tail tip using a glucometer (Glucofix Premium, Menarini Diagnostic) on semi-fasted animals (4 hours of fasting).

### METABOpack™ Session

In the present project, a METABOpack™ session was consisted of a 12-hour period of acclimation followed by a 48-hour period of recording of the respiratory exchanges, FI, water intake (WI) and locomotor activity of each animal.

Animals were removed from their housing cages, weighed and then individually placed into the physiocages (home cages of the Physiocage system) for 12 hours, which were allowed the animals to acclimatize and accustom to this new housing condition.

Following this acclimation period, the parameters cited above were automatically and simultaneously recorded for 48 hours, except for body weight that was measured manually every day.

The METABOpack™ sessions were conducted at the start (T₁₋₂), the middle (T₁₄₋₁₅) and the end (T₂₆₋₂₇) of the treatment period.

It is important to remember that 8 mice per experimental groups were submitted to the METABOpack™ sessions. In this context, and considering that the Physiocage System was composed of only 8 physiocages, the study was split into 6 successive arms of 8 mice each (arm 1 to 6). Each arm was composed of 1-2 animals of each experimental group.

### METABOpack™ Recorded Parameters:

Cumulative food intake (g)

Cumulative water intake (ml)

Horizontal spontaneous activity & Rearing (arbitrary unit)

VO2 (ml/day/kg lean)

VCO2 (ml/day/kg lean)

### Analyses

### Meal Pattern

Meal pattern analysis was performed on high-resolution recordings of food intake obtained during the METABOpack™ sessions. The Physiocage system allowed recording food intake with an accuracy of 1 measure per second and a precision of 0.02 g. The physiocages were equipped with a system allowing to precisely collect the spillage of the animals, by the mean of a platform located under the food dispenser. This platform and the spillages it contains, were permanently weighted by the system and the values added to the total weight of the food dispenser. With this system, the progressive decrease in the food dispenser net weight only reflected the food consumption of the animals. The analysis was performed by plotting data every minute. Each weight variation of the feeding container (transition from one stable value to another) was considered as a “bout”, with each bout separated from each other bout by an “inter-bout interval”. A meal consisted of at least one bout, with each meal separated from each other meal by a “post-meal interval”. For the purpose of this study, a meal was defined as any feeding episode causing a change in food weight of 0.02 g and separated by at least 5 min from any subsequent episode. Based on this criterion, the following parameters were calculated for each experimental group:

mean first meal latency (min)

mean meal size (g)

mean meal frequency

mean meal duration (min)

mean post-meal interval (min)

mean Eating Rate (ER, g/min)

mean Satiety Ratio (SR, min/g)

### Indirect Calorimetry

The Physiocage System allows recording the O2 consumption (VO₂) and the CO2 production (VCO2) by indirect calorimetry. For each animal, both VO2 and VCO2 were recorded for 3 min every 30 minutes and the mean values were calculated by the system.

Energy Expenditure (EE) and Respiratory Exchange Ratio (RER) were calculated with formulas that are based on VO2 and VCO2 values:

EE (Kcal/day/Kg lean)=(3.815+(1.232×VCO2/VO2))×VO2×1.44

RER=VCO2/VO2

Analysis of these parameters was performed as follows: for each mouse, the mean values were calculated from the corresponding recordings for each nycthemeral phase (Diurnal Phase or DP, Nocturnal Phase or NP and Whole day or WD). The means per nycthemeral phase were then calculated for each group for statistical comparisons.

### Horizontal Spontaneous Activity and Rearing Behavior

Horizontal spontaneous activity and rearing behavior were recorded every second by the Physiocage System. The analysis was performed by plotting data every 30 minutes in order to obtain the kinetic of the behaviors. For each mouse, the sums of the activity and rearing events were calculated for each nycthemeral phase (Diurnal period or DP; Nocturnal period or NP and Whole Day or WD). The means per nycthemeral phase were then calculated for each group for statistical comparisons.

### Terminal Blood and Tissue Collection

On T₂₈, after 4 hours of fasting, a terminal sampling of blood and organs/tissues was performed on anaesthetized mice (by a mixture of Ketamine/Xylazine, 80/10 mg/kg). A large blood sample was collected by cardiac puncture, and the following organ/tissues were collected, weighed, snap-frozen in liquid nitrogen and stored at −80° C. for further analyses:

Fat depots: epididymal WAT, subcutaneous WAT, and interscapular BAT

Liver

Muscle (Gastrocnemius muscle)

Heart

The blood samples were collected into centrifuge tubes pre-filled with heparin. Plasma was separated by centrifugation (3000×g, 15 min, 4° C.), collected and stored at −80° C. for further analyses.

### Statistical Analyses

The whole data were represented as Mean±SEM. Statistical analyses were performed with the Statview 5.0.1 program (Statview software, Cary, N.C., USA). Data were analyzed by one-way ANOVA or repeated-measures ANOVA. For comparisons of the mean value of a given parameter between the experimental groups, data were analyzed using one-way ANOVA, except for body weight, body weight gain, food intake and cumulative food and water intakes where repeated-measures ANOVA was used. When both types of ANOVA analyses revealed a significant effect, a posthoc analysis was performed using a Fisher's PLSD test.

Analyses of the meal pattern, total spontaneous activity, rearing and respiratory exchanges and related parameters were performed as follows: for each mouse, the mean values were calculated from the corresponding recordings for each nychtemeral phase (Whole day or WD, Diurnal Period or DP, Nocturnal Period or NP). The means per nycthemeral phase were then calculated for each group for statistical comparisons.

### Impact of 89BIO-100 on Food Intake and Body Weight

### Impact of 89BIO-100 on Food Intake and Cumulative Food Intake

Apart from the METABOpack sessions, the food intake of the different experimental groups was daily measured when the animals were housed in their individual cages, between T₃-T₁₃ and T₁₆-T₂₅. Repeated measures ANOVA analyses did not reveal any significant difference in daily food intake and cumulative food intake between the experimental groups (P=0.0706 and P=0.0914, respectively).

General Methods: Naive CD-1 mice were treated subcutaneously (SC) for 4 weeks at ambient temperature with BIO89-100 at doses of 0, 0.3, 1.0 and 3.0 mg/kg (3qW×4 weeks) or with liraglutide at 0.2 mg/kg (BID×4 W; SC). BW and FC were measured daily throughout the treatment period. To investigate the impact of BIO89-100 on energy expenditure, the respiratory exchanges (VO₂ and VCO₂) were recorded during 3 distinct sessions of METABOpack™, a multiparametric approach allowing an automated and simultaneous recording of FC, water intake, respiratory exchanges and spontaneous activity. Meal pattern analysis was conducted on the high-resolution recordings of FC. The body composition (fat, lean and fluid masses) was analyzed 3 times during the study by the mean of Time-Domain Nuclear Magnetic Resonance (TD-NMR).

### Results:

Impact of 89BIO-100 on body weight and delta body weight The body weight of the different experimental groups was daily measured over the treatment period (between T₁ and T₂₈). As shown in FIG. 1A repeated measures using ANOVA analysis revealed a significant difference in body weight between the experimental groups (F=4.735, P=0.0030). As expected, post-hoc analyses showed a significant decrease in body weight of mice treated with the benchmark, Liraglutide when compared to the vehicle group over the treatment period, except on T₁, T₅, T₆, T₁₂, T₂₅ and T₂₈ while no difference was observed. Moreover, mice treated with the highest dose of 89BIO-100 (3 mg/kg) showed a lower body weight, when compared to the vehicle group, except on T₂, T₃ and T₄. On the other hand, mice treated with the lowest and intermediate dose of 89BIO-100 (0.3 and 1 mg/kg) did not show any difference in body weight when compared to the vehicle group.

Mice treated with the lowest dose of 89BIO-100 (0.3 mg/kg) exhibited a greater body weight over the treatment period (from T₂ to T₂₄ and on T₂₆), than those treated with Liraglutide, whereas the intermediate dose of 89BIO-100 (1 mg/kg) showed only a greater body weight at the beginning of the treatment period (from T₂ to T₅), when compared to the Liraglutide group. The body weight of mice treated with the highest dose of 89BIO-100 (3 mg/kg) exhibited a lower body weight during a few days in the middle of the treatment period (from T₁₁ to T₁₃) when compared to the Liraglutide group. Finally, treatment with the highest dose of 89BIO-100 (3 mg/kg), reduced the body weight of mice over the treatment period when compared to mice treated with the dose of 0.3 mg/kg and 1 mg/kg, respectively.

Regarding the delta body weight (g), and as shown in FIG. 1B, the repeated measures ANOVA analysis reveals a significant difference in delta body weight between experimental groups (F=5.648, P=0.0010). Post-hoc analyses showed a significant decrease in delta body weight of the Liraglutide group on T₂, T₃, T₁₄ to T₁₆ and T₂₁, when compared to the vehicle group. The lowest dose of 89BIO-100 (0.3 mg/kg) induced an increase in delta body weight when compared to the vehicle group only from T₃ to T₆. In contrast, mice treated with the intermediate and highest dose of 89BIO-100 (1 and 3 mg/kg) showed a marked decrease in delta body weight, when compared to the vehicle group, from T₈ to T₂₁, on T₂₃, T₂₄ and T₃ and from T₆ to T₂₈, respectively.

Mice treated with the lowest dose of 89BIO-100 (0.3 mg/kg) showed an increase in delta body weight than those treated with Liraglutide from T₂ to T₁₁ and from T₁₃ to T₁₆. Moreover, mice treated with the intermediate and highest dose of 89BIO-100 (1 and 3 mg/kg) showed an increase in delta body weight at the beginning of the treatment period (from T₂ to T₄), when compared to the Liraglutide group. However, mice treated with 89BIO-100 at the dose of 3 mg/kg showed a significant decrease in delta body weight from T₈ to T₁₃ and on T₂₅, when compared to the Liraglutide group.

Finally, mice treated with the 89BIO-100 1 mg/kg exhibited a significant decrease in delta body weight than those treated with 89BIO-100 0.3 mg/kg from T₆ to T₂₁. Mice treated with the highest dose at 3 mg/kg exhibited a marked decrease in delta body weight from T₅ to T₂₈, when compared to those treated with the dose of 0.3 mg/kg and also a significant decrease in delta body weight on T₁₁, T₁₂, T₁₃ and T₁₅, when compared to those treated with the dose of 1 mg/kg.

During the three (3) METABOpack sessions (T₁/T₂, T₁₄/T₁₅ and T₂₆/T₂₇), cumulative food intake and cumulative water intake were evaluated. Moreover, meal pattern analysis was performed on high-resolution recordings of food intake obtained during the METABOpack™ sessions, in order to investigate the impact of 89BIO-100 on the following parameters: first meal latency (min), mean meal size (g), mean meal frequency, mean meal duration (min), mean post-meal interval (min), mean eating rate (g/min) and mean satiety ratio (min/g).

Impact of 89BIO-100 on Cumulative Food Intake and Meal Pattern on T₁/T₂

On T₁

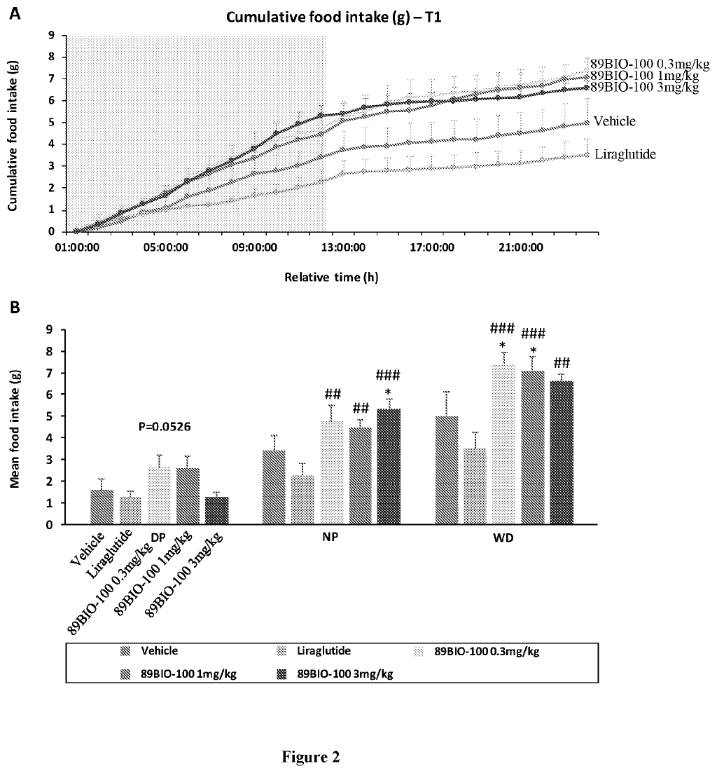

As shown in FIG. 2A, the repeated measures ANOVA analysis reveals a significant difference in the one hour-resolution recordings of the cumulative food intake between experimental groups on T₁ (F=6.001; P=0.0010). Surprisingly, post-hoc analyses did not reveal significant difference in cumulative food intake between the Liraglutide group and the Vehicle group. In contrast, post-hoc analyses showed a significant increase in cumulative food intake with the treatment 89BIO-100 at the three doses, when compared to the vehicle group. In details, when compared to the vehicle group, the cumulative food intake of mice treated with the dose of 0.3 mg/kg was increased at 5 h, 6 h, and from 15 h to 24 h, also the cumulative food intake of mice treated with the dose of 1 mg/kg was increased at 1 h, 3 h, and from 18 h to 24 h and finally the cumulative food intake of mice treated with the dose of 3 mg/kg was increased from 10 h to 12 h and from 14 h to 17 h.

Mice treated with the three doses of 89BIO-100 exhibited an increase in cumulative food intake from 5 h to 24 h, than those treated with the Liraglutide. Nevertheless, there was no significant difference between the three 89BIO-100 treated groups.

For reason of clarity, statistical differences in cumulative food intake between experimental groups on T₁ were presented as a form of tables (FIG. 2A).

Regarding the analyses of food intake on T₁ per nycthemeral and as shown in FIG. 2B, no significant difference was observed in mean food intake during DP (diurnal phase) of T₁ (close to the significance: p=0.0526).

During the NP (nocturnal phase) of T₁, the mean food intake of the highest dose of 89BIO-100 (3 mg/kg) was only higher than those of the vehicle group. During the WD (whole day), the mean food intake of the lowest and the intermediate dose of 89BIO-100 (0.3 and 1 mg/kg) was increased, when compared to the vehicle group.

Moreover, mice treated with 89BIO-100 (whatever the dose considered) showed an increase in mean food intake, when compared to the Liraglutide group during the NP and the WD.

No significant difference in mean food intake was observed between the three 89BIO-100 treated groups during both the NP and the WD.

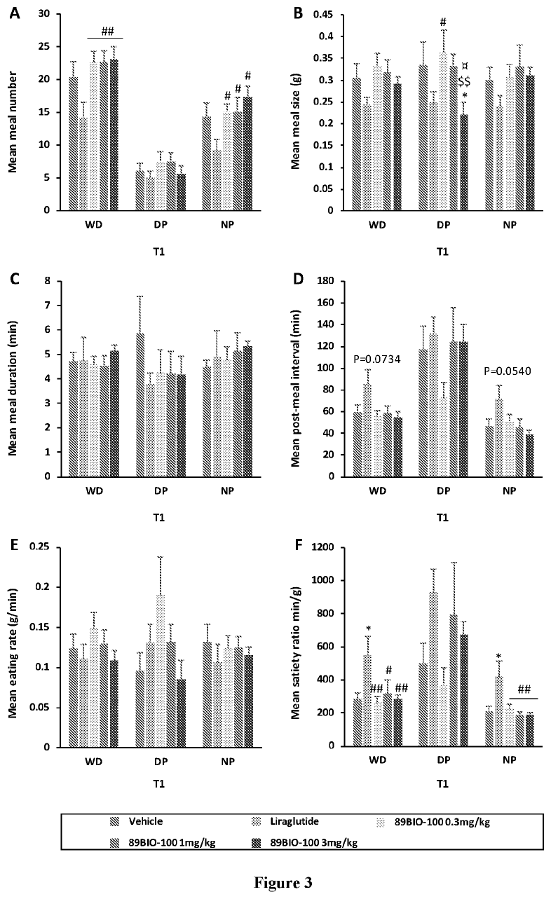

Regarding the meal pattern and as shown in FIGS. 3A-3F, meal pattern analysis on T₁ revealed that during the WD and NP, the mean satiety ratio of the Liraglutide group was increased, when compared to the vehicle group. During the DP, the mean meal size of 89BIO-100 3 mg/kg group was reduced, when compared to the vehicle group.

Moreover, meal pattern analysis on T₁ revealed that during the WD and NP, the three doses of 89BIO-100 increased the mean meal number and decreased the mean satiety ratio, when compared to the Liraglutide group. During the DP, the dose of 0.3 mg/kg increased the mean meal size, when compared to the Liraglutide group.

Finally, during the DP, the dose of 3 mg/kg decreased the mean meal size, when compared to the dose of 0.3 and 1 mg/kg, respectively.

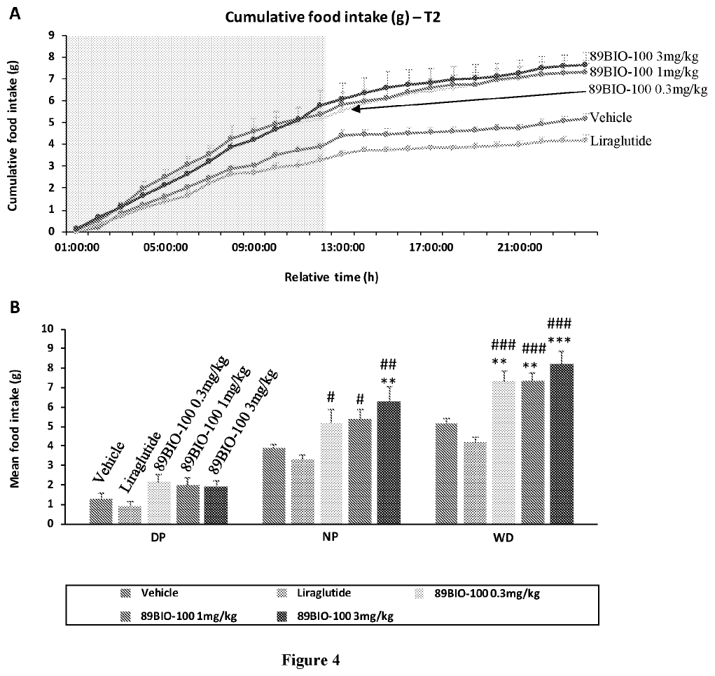

As shown in FIG. 4A, the repeated measurements using ANOVA analysis reveal a significant difference in the one-hour resolution recordings of the cumulative food intake between experimental groups at T₂ (F=6.772; p=0.0003). Post-hoc analyses did not reveal a significant difference in cumulative food intake between the Liraglutide group and the vehicle group. In contrast, post-hoc analyses showed an increase in cumulative food intake with the treatment 89BIO-100 at the dose of 0.3 and 1 mg from 13 h to 24 h, when compared to the vehicle group. With the dose of 3 mg/kg, the cumulative food intake was increased over 24 h, except at 4 h.

The three doses of 89BIO-100 increased the cumulative food intake on T₂, when compared to the Liraglutide group. More specifically, the cumulative food intake in mice treated with 89BIO-100 at the dose of 0.3 mg/kg was increased at 5 h, 6 h and from 10 h to 24 h, the cumulative food intake in mice treated with 89BIO-100 at the dose of 1 mg/kg was increased from 4 h to 24 h and the cumulative food intake in mice treated with 89BIO-100 at the dose of 3 mg/kg was increased over 24 h, except at 4 h, when compared to the Liraglutide group.

Finally, the cumulative food intake of mice treated with 89BIO-100 at the dose of 3 mg/kg was slightly increased at 1 h and 2 h only, when compared to mice treated with 89BIO-100 at the dose of 0.3 mg/kg. In contrast, no significant difference in cumulative food intake was observed between 89BIO-100 treated groups at the dose of 1 and 3 mg/kg and between the dose of 0.3 and 1 mg/kg.

Regarding the analyses of food intake on T₂ per nycthemeral and as shown in FIG. 4B, no significant difference was observed in mean food intake during the DP of T₂.

During the NP (nocturnal phase) of T₂, the mean food intake of the highest dose of 89BIO-100 (3 mg/kg) was only higher than those of the vehicle group. During the WD (whole day), the mean food intake of the three doses of 89BIO-100 was increased, when compared to the vehicle group.

Moreover, mice treated with 89BIO-100 (whatever the dose considered) showed an increase in mean food intake, when compared to the Liraglutide group during the NP and the WD.

No significant difference in mean food intake was observed between the three 89BIO-100 treated groups during the NP and the WD.

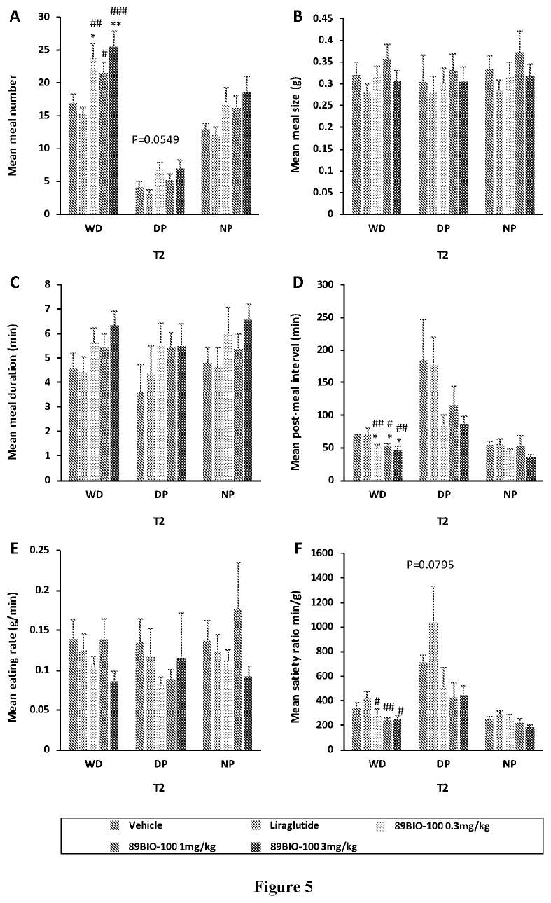

Regarding the meal pattern and as shown in FIGS. 5A-5F, meal pattern analysis on T₂ revealed that only during the WD, the mean meal number of the 89BIO-100 at the dose of 0.3 and 3 mg/kg was increased, when compared to the vehicle group. During the same nycthemeral phase, the mean post-meal interval of the three doses of 89BIO-100 was shorter than that of the vehicle group.

Moreover, meal pattern analysis on T₂ revealed that during the WD only, the three doses of 89BIO-100 increased the mean meal number and decreased the mean post-meal interval and satiety ratio, when compared to the Liraglutide group.

### Impact of 89BIO-100 on Cumulative Water Intake at T1/T2

On T1

Repeated measurements using ANOVA analysis did not reveal any significant difference in one-hour resolution recordings of the cumulative water intake between experimental groups on T₁ (F=2.41; p=0.0689, close to the significance).

Regarding the analyses of water intake on T₁ per nycthemeral, the mean water intake of the Liraglutide group was decreased during the NP and WD of T₁, when compared to the vehicle group. However, no significant difference was observed between the three doses of 89BIO-100 groups and vehicle group.

Moreover, mice treated with 89BIO-100 (whatever the dose considered) showed an increase in mean water intake, when compared to the Liraglutide group during the NP and the WD. The dose of 0.3 and 1 mg/kg also increased the mean water intake during the DP, when compared to the Liraglutide group.

During the DP, the mean water intake of the 89BIO-100 3 mg/kg group was decreased, when compared to the dose of 0.3 and 1 mg/kg, respectively.

Repeated measurements using ANOVA analysis revealed a significant difference in one-hour resolution recordings of the cumulative water intake between the experimental groups on T₂ (F=6.319; p=0.0005). Surprisingly, post-hoc analyses did not reveal a significant difference in water intake between the Liraglutide group and the vehicle group over 24 h. However, the treatment with 89BIO increased the cumulative water intake, when compared to the vehicle group. Indeed, the cumulative water intake in mice treated with 89BIO-100 0.3 mg/kg was increased at 5 h, 6 h, 7 h, 9 h, 10 h and from 12 h to 24 h, the cumulative water intake in mice treated with 89BIO-100 1 mg/kg was increased from 4 h to 24 h and the cumulative water intake in mice treated with 89BIO-100 3 mg/kg was increased from 2 h to 24 h.

Moreover, the three doses of 89BIO-100 increased the cumulative water intake, when compared to the Liraglutide group. More precisely, the cumulative water intake of mice treated with 89BIO-100 0.3 mg/kg was increased from 5 h to 24 h, the cumulative water intake of mice treated with 89BIO-100 1 mg/kg was increased from 4 h to 24 h and the cumulative water intake of mice treated with 89BIO-100 3 mg/kg was increased from 2 h to 24 h, when compared to the Liraglutide group.

Finally, no significant difference was observed in cumulative water intake on T₂ between the three 89BIO-100 treated groups.

Regarding the analyses of water intake on T₂ per nycthemeral, no significant difference in mean water intake was observed between the Liraglutide and vehicle groups, whatever the phase considered. However, the mean water intake was increased in mice treated with 89BIO-100 (whatever the dose considered) during the WD, and increased in mice treated with the dose of 0.3 and 1 mg/kg during the NP, when compared to the vehicle group.

Moreover, mice treated with 89BIO-100 (whatever the dose considered) showed an increase in mean water intake, when compared to the Liraglutide group, whatever the phase considered.

Impact of 89BIO-100 on Cumulative Food Intake and Meal Pattern at T₁₄/T₁₅

On T₁₄

Repeated measurements using ANOVA analysis did not reveal any significant difference in one-hour resolution recordings of the cumulative food intake between experimental groups at T₁₄ (F=0.987; p=0.4256).

Regarding the meal pattern, meal pattern analysis at T₁₄ did not reveal any significant difference in mean meal number, mean meal size, mean meal duration, mean post-meal interval, mean eating rate and mean satiety ratio between the experimental groups.

Meal pattern analysis of the first meal on T₁₄ revealed a decrease in mean post-meal interval of the Liraglutide group, when compared to the vehicle group. Mice treated with 89BIO-100 at the dose of 3 mg/kg showed a decrease in mean post-meal interval and mean satiety ratio, when compared to the vehicle group. Then, mice treated with 89BIO-100 at the dose of 0.3 mg/kg showed a reduction in mean post-meal interval, when compared to the Vehicle group.

Moreover, the treatment with 89BIO-100 at the dose of 1 mg/kg showed an increase in mean post-meal interval, when compared to the Liraglutide group.

Finally, the mean satiety ratio of 89BIO-100 1 mg/kg group was higher than that of 89BIO-100 0.3 mg/kg group. Mice treated at the dose of 3 mg/kg showed a reduction in mean post-meal interval and mean satiety ratio, when compared to those treated at the dose of 1 mg/kg.

On T₁₅

Repeated measurements using ANOVA analysis did not reveal any significant difference in one-hour resolution recordings of the cumulative food intake between experimental groups on T₁₅ (F=2.040; P=0.1115).

Regarding the analyses of food intake on T₁₅ per nycthemeral, the mean food intake during the WD was only increased with the treatment of 89BIO-100 (whatever the dose considered), when compared to the vehicle group.

Moreover, the mean food intake during the WD was only increased with the treatment of 89BIO-100 (whatever the dose considered), when compared to the Liraglutide group.

Meal pattern analysis of the first meal on T₁₅ revealed a decrease in mean first meal latency and in mean post-meal interval of the Liraglutide group, when compared to the vehicle group. Mice treated with 89BIO-100 at the dose of 3 mg/kg showed a decrease in mean post-meal interval, when compared to the vehicle group. Then, the mean first meal latency of mice treated with the three doses of 89BIO-100 was reduced when compared to the vehicle group.

Moreover, the treatment with 89BIO-100 at the dose of 1 mg/kg showed an increase in mean post-meal interval, when compared to the Liraglutide group.

Finally, the mean post-meal interval of mice treated with 89BIO-100 at the dose of 3 mg/kg was lower than that of mice treated with 89BIO-100 at the dose of 1 mg/kg.

Impact of 89BIO-100 on Cumulative Water Intake on T₁₄/T₁₅

On T₁₄

Repeated measurements using ANOVA analysis revealed a significant difference in one-hour resolution recordings of the cumulative water intake between the experimental groups on T₁₄ (F=6.425; P=0.0004). Post-hoc analyses did not reveal any significant difference in water intake between the Liraglutide group and the Vehicle group over 24 h. However, both doses of 1 and 3 mg/kg increased the cumulative water intake of mice from 2 h to 24 h, when compared to the vehicle group.

Moreover, both doses of 1 and 3 mg/kg increased the cumulative water intake of mice from 2 h to 24 h, when compared to the Liraglutide group.

Finally, the dose of 1 mg/kg increased the cumulative water intake of mice at 2 h and from 7 h to 24 h, when compared to the mice treated with the dose of 0.3 mg/kg while the dose of 3 mg/kg increased the cumulative water intake of mice from 3 h to 24 h, when compared to the dose of 0.3 mg/kg.

Regarding the analyses of water intake on T₁₄ per nycthemeral, no significant difference in mean water intake was observed between the Liraglutide and vehicle groups, whatever the phase considered. However, the mean water intake was increased in mice treated with 89BIO-100 at the dose of 1 and 3 mg/kg, when compared to the vehicle group, whatever the phase considered.

Moreover, mice treated with 89BIO-100 at the dose of 1 and 3 mg/kg showed an increase in mean water intake, when compared to the Liraglutide group, whatever the phase considered.

Finally, both doses of 1 and 3 mg/kg increased the mean water intake during the NP, when compared to the dose of 0.3 mg/kg. The dose of 3 mg/kg also increased the mean water intake of mice during the WD, when compared to the dose of 0.3 mg/kg.

On T₁₅

Repeated measurements using ANOVA analysis revealed a significant difference in one-hour resolution recordings of the cumulative water intake between the experimental groups on T₁₅ (F=5.500; P=0.0017). Post-hoc analyses did not reveal any significant difference in water intake between the Liraglutide group and the vehicle group over 24 h. However, both doses of 1 and 3 mg/kg increased the cumulative water intake of mice over 24 h, when compared to the vehicle group.

Moreover, both doses of 1 and 3 mg/kg increased the cumulative water intake of mice over 24 h, when compared to the Liraglutide group.

Finally, the dose of 1 mg/kg increased the cumulative water intake of mice from 2 h to 24 h, when compared to the dose of 0.3 mg/kg while the dose of 3 mg/kg increased the cumulative water intake of mice from 1 h to 15 h, when compared to the dose of 0.3 mg/kg. Regarding the analyses of water intake on T₁₄ per nycthemeral, no significant difference in mean water intake was observed between the Liraglutide and vehicle groups, whatever the phase considered. However, the mean water intake was increased in mice treated with 89BIO-100 at the dose of 1 and 3 mg/kg, when compared to the vehicle group, during the NP and the WD.

Moreover, mice treated with 89BIO-100 at the dose of 1 and 3 mg/kg showed an increase in mean water intake, when compared to the Liraglutide group, during the NP and the WD.

Finally, both doses of 1 and 3 mg/kg increased the mean water intake during the NP, when compared to the dose of 0.3 mg/kg. The dose of 1 mg/kg also increased the mean water intake of mice during the WD, when compared to the dose of 0.3 mg/kg.

Impact of 89BIO-100 on Cumulative Food Intake and Meal Pattern on T₂₆/T₂₇

On T₂₆

Repeated measurements using ANOVA analysis did not reveal any significant difference in one-hour resolution recordings of the cumulative food intake between experimental groups on T₂₆ (F=0.643; P=0.6351).

Considering the analyses of food intake on T₂₆ per nycthemeral, no significant difference was observed in mean food intake between experimental groups on T₂₆, whatever the time period considered (DP, NP and WD).

Regarding the meal pattern, meal pattern analysis on T₂₆ did not reveal any significant difference in mean meal number, mean meal size, mean meal duration, mean post-meal interval, mean eating rate and mean satiety ratio between the experimental groups.

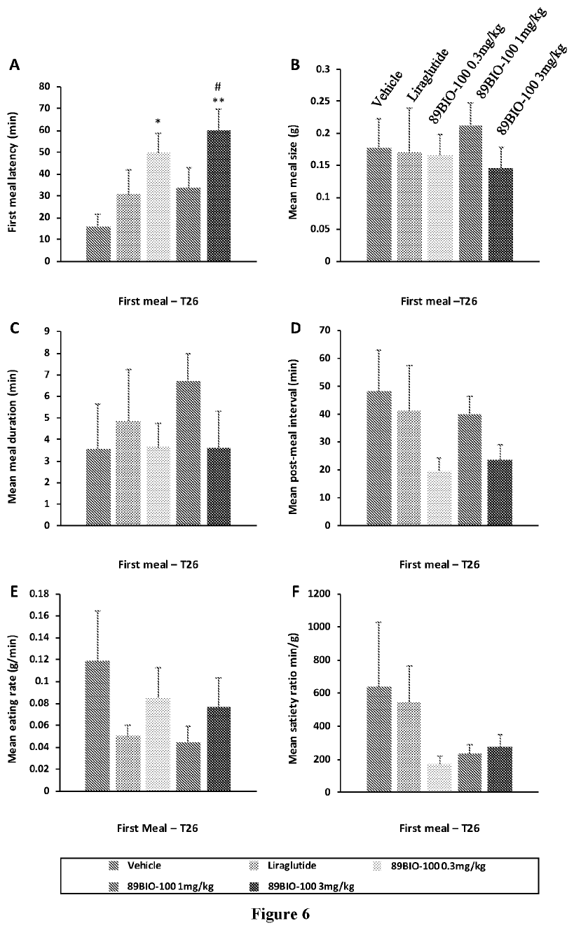

As shown in FIGS. 6A-6F, meal pattern analysis of the first meal on T₂₆ revealed an increase in mean first meal latency of both doses of 0.3 and 1 mg/kg-treated mice, when compared to the vehicle group.

Moreover, the treatment with 89BIO-100 at the dose of 3 mg/kg showed an increase in mean first meal latency in mice, when compared to the Liraglutide group.

On T₂₇

Repeated measurements using ANOVA analysis did not reveal any significant difference in one-hour resolution recordings of the cumulative food intake between the experimental groups on T₂₇ (F=0.362; P=0.8344).

Considering the analyses of food intake on T₂₇ per nycthemeral, no significant difference was observed in mean food intake between experimental groups on T₂₇, whatever the time period considered (DP, NP and WD).

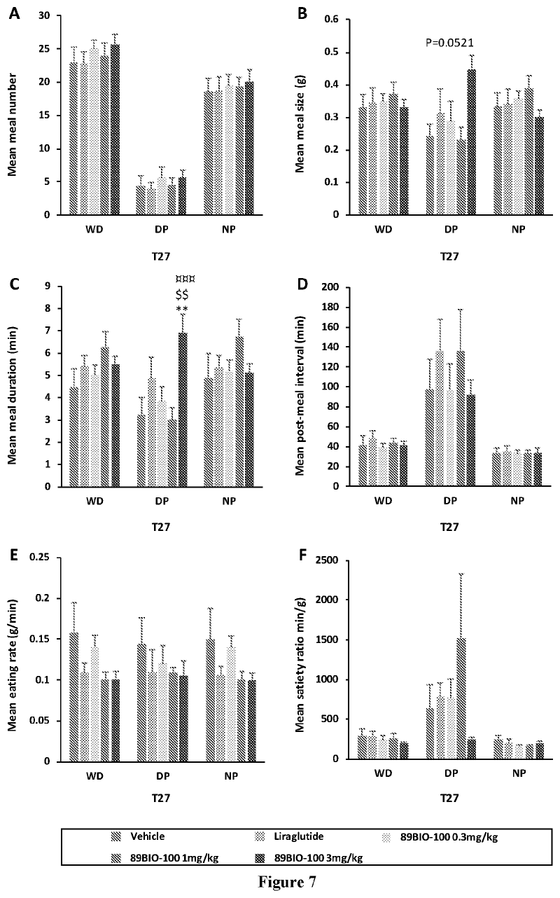

Regarding the meal pattern and as shown in FIGS. 7A-7F, meal pattern analysis on T₂₇ revealed a significant increase in mean meal duration at the dose of 3 mg/kg, when compared to the vehicle, 89BIO-100 at the dose of 0.3 and 1 mg/kg groups, respectively. Meal pattern analysis of the first meal on T₂₇ did not reveal any significant difference in mean first meal latency, mean meal size, mean meal duration, mean post-meal interval, meal eating rate and mean satiety ratio between the experimental groups.

Impact of 89BIO-100 on Cumulative Water Intake on T₂₆/T₂₇

On T₂₆

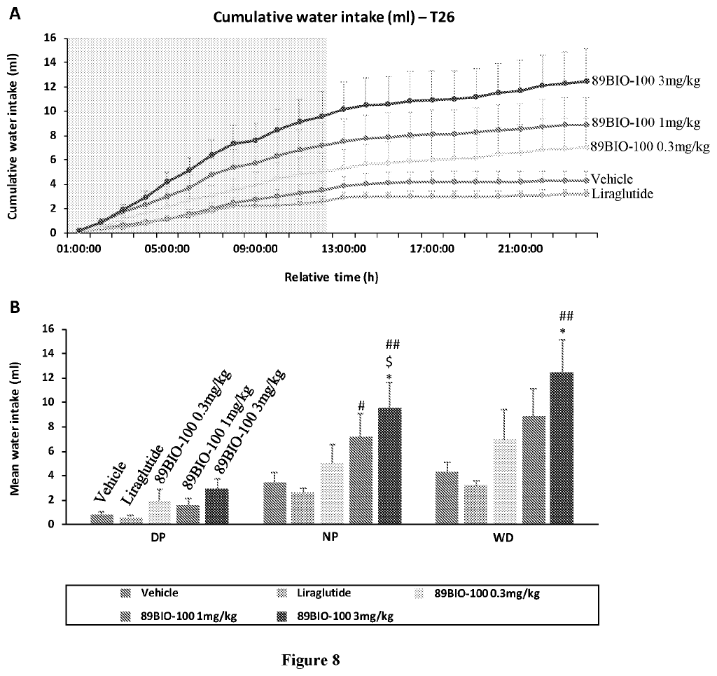

As shown in FIG. 8A, the repeated measurements using ANOVA analysis revealed a significant difference in one-hour resolution recordings of the cumulative water intake between experimental groups on T₂₆ (F=2.872; P=0.0350). Post-hoc analyses did not reveal any significant difference in water intake between the Liraglutide group and the vehicle group over 24 h. However, the treatment 89BIO-100 at the dose of 1 mg/kg increased the cumulative water intake of mice only at 3 h and 4 h, when compared to the vehicle group while the dose of 3 mg/kg increased the cumulative water intake from 3 h to 24 h, when compared to the vehicle group.

Moreover, the treatment 89BIO-100 at the dose of 1 mg/kg increased the cumulative water intake of mice at 3 h, 4 h, and from 6 h to 21 h, when compared to the Liraglutide group while the dose of 3 mg/kg increased the cumulative water intake of mice from 3 h to 24 h, when compared to the Liraglutide group.

Finally, the dose of 3 mg/kg increased the cumulative water intake of mice from 5 h to 16 h, when compared to the dose of 0.3 mg/kg.

Regarding the analyses of water intake on T₂₆ per nycthemeral and as shown in FIG. 8B, no significant difference in mean water intake was observed between the Liraglutide and vehicle groups, whatever the phase considered. However, the mean water intake was increased in mice treated with 89BIO-100 at the dose of 3 mg/kg, when compared to the vehicle group, during the NP and the WD.

Moreover, mice treated with 89BIO-100 at the dose of 1 and 3 mg/kg showed an increase in mean water intake during the NP, while mice treated with 89BIO-100 at the dose of 3 mg/kg showed also an increase in mean water intake during the WD, when compared to the Liraglutide group.

Finally, the dose of 3 mg/kg increased the mean water intake during the NP, when compared to the dose of 0.3 mg/kg.

On T₂₇

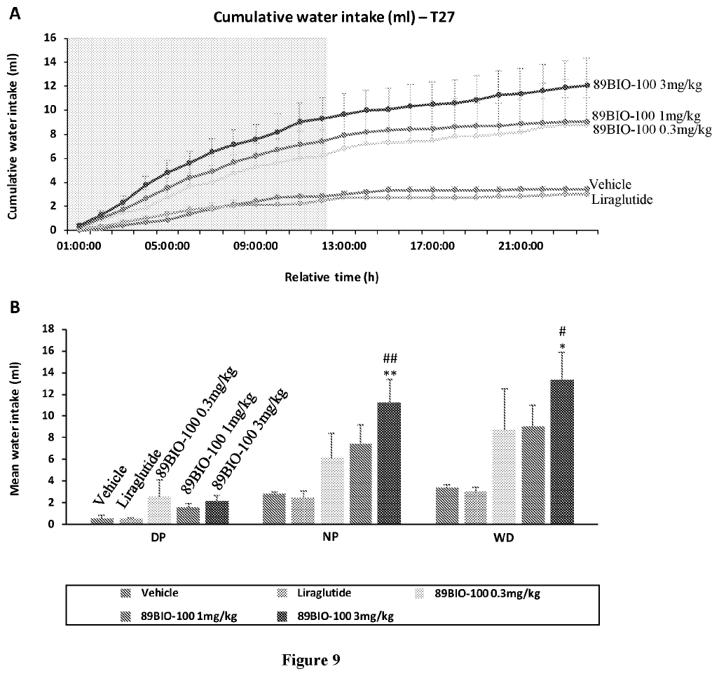

As shown in FIG. 9A, the repeated measurements using ANOVA analysis revealed a significant difference in one-hour resolution recordings of the cumulative water intake between the experimental groups on T₂₇ (F=3.031; P=0.0284). Post-hoc analyses did not reveal any significant difference in water intake between the Liraglutide group and the vehicle group over 24 h. However, the treatment 89BIO-100 at the dose of 1 mg/kg increased the cumulative water intake of mice from 2 h to 5 h, while the dose of 3 mg/kg increased the cumulative water intake of mice over 24 h, when compared to the Vehicle group.

Moreover, the treatment 89BIO-100 at the dose of 3 mg/kg increased the cumulative water intake of mice over 24 h, when compared to the Liraglutide group.

Finally, the dose of 3 mg/kg increased the cumulative water intake of mice at 1 h and from 3 h to 8 h, when compared to the dose of 0.3 and at 3 h, 4 h, 5 h and 7 h, when compared to the dose of 1 mg/kg.

Regarding the analyses of water intake on T₂₇ per nycthemeral and as shown in FIG. 9B, no significant difference in mean water intake was observed between the Liraglutide and vehicle groups, whatever the phase considered. However, the mean water intake was increased in mice treated with 89BIO-100 at the dose of 3 mg/kg during the NP and the WD, when compared to the vehicle and Liraglutide groups, respectively.

Impact of 89BIO-100 on respiratory exchanges and related parameters During the three (3) METABOpack sessions (T₁/T₂, T₁₄/T₁₅ and T₂₆/T₂₇), respiratory exchanges, i.e. oxygen consumption (VO₂) and carbon dioxide production (VCO₂) were evaluated in order to assess of the impact of 89BIO-100 on energy expenditure (EE) and respiratory exchange ratio (RER) in CD-1 mice at ambient temperature.

Impact of 89BIO-100 on Respiratory Exchanges and Related Parameters on T₁/T₂

On T₁

Regarding the analyses of respiratory exchanges on T₁ per nycthemeral, no significant difference in VO₂, and EE was observed between the experimental groups on T₁, whatever the time period considered.

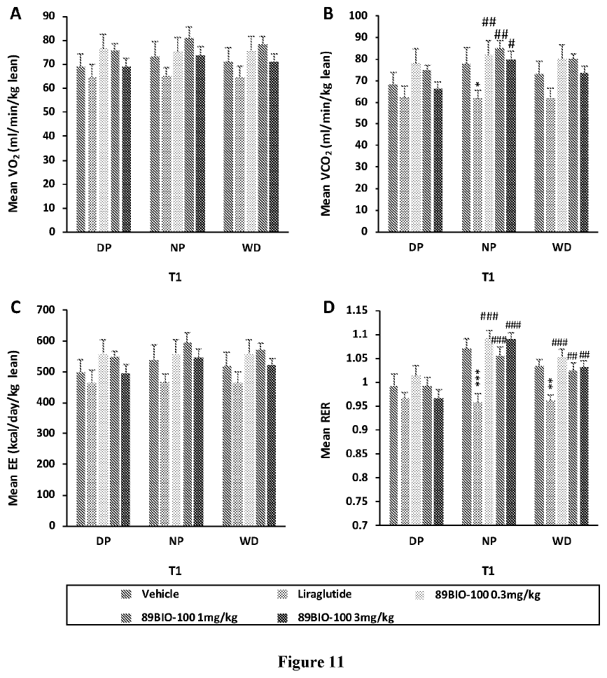

Regarding the VCO₂ and as shown FIG. 11B, the VCO₂ of mice treated with Liraglutide was decreased during the NP only, when compared to the vehicle group. No significant difference in VCO₂ was observed between the treatment with 89BIO-100 (whatever the dose considered), when compared to the vehicle group.

Moreover, mice treated with 89BIO-100 (whatever the dose considered) showed an increase in VCO₂ during the NP, when compared to the Liraglutide group.

Finally, no significant difference in VCO₂ was observed between all the 89BIO-100 treated groups.

Regarding the RER and as shown in FIGS. 10B and 11D, the RER of mice treated with Liraglutide was decreased during the NP and the WD, when compared to the vehicle group. No significant difference in RER was observed between the treatment with 89BIO-100 (whatever the dose considered), when compared to the vehicle group.

Moreover, mice treated with 89BIO-100 (whatever the dose considered) showed an increase in RER during the NP and the WD, when compared to the Liraglutide group.

Finally, no significant difference in RER was observed between the 89BIO-100 treated groups.

On T₂

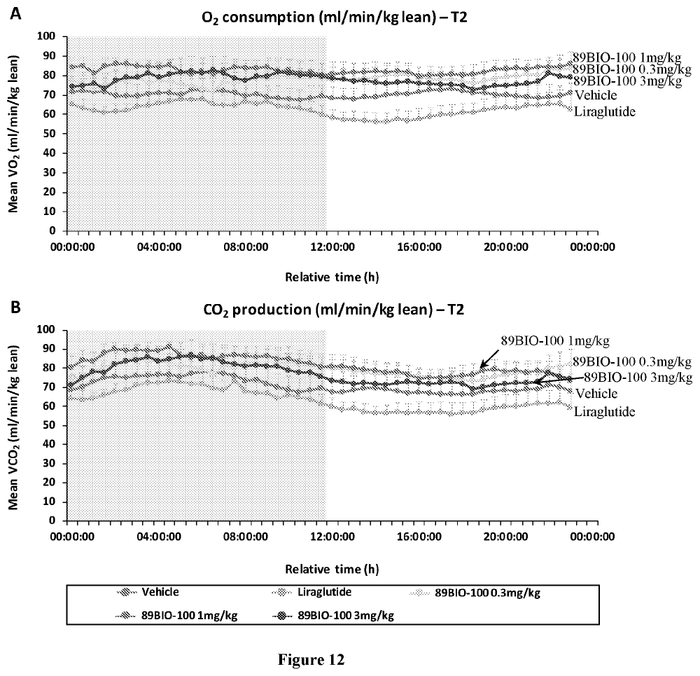

Regarding the analyses of respiratory exchanges on T₂ per nycthemeral and as shown in FIGS. 12B/14B and 13B/14D, no significant difference in VCO₂, and RER was observed between the experimental groups on T₂, whatever the time period considered.

Regarding the VO₂ and as shown in FIGS. 12A/14A, no significant difference in VO₂ was observed between the Liraglutide and the vehicle groups. No significant difference in VO₂ was observed between mice treated with 89BIO-100 (whatever the dose considered) and mice treated with the Vehicle.

Moreover, mice treated with 89BIO-100 (whatever the dose considered) showed an increase in VO₂ during the DP and the WD, when compared to the Liraglutide group.

Finally, no significant difference in VO₂ was observed between the three 89BIO-100 treated groups.

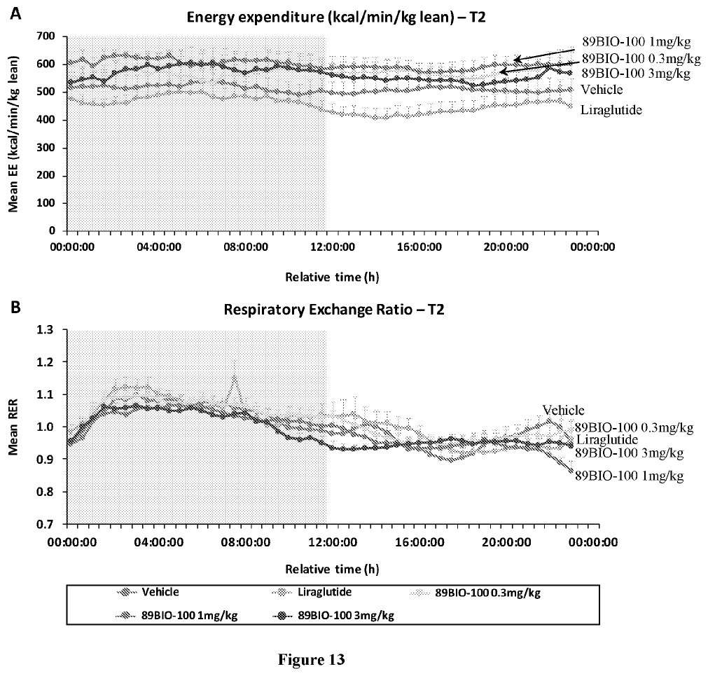

Regarding the EE and as shown in FIGS. 13A/14C, no significant difference in EE was observed between the Liraglutide and the vehicle groups. No significant difference in EE was observed between mice treated with 89BIO-100 (whatever the dose considered) and mice treated with the Vehicle.

Moreover, mice treated with 89BIO-100 (whatever the dose considered) showed an increase in EE during the DP and the WD, when compared to the Liraglutide group.

Finally, no significant difference in EE was observed between the 89BIO-100 treated groups themselves.

Impact of 89BIO-100 on Respiratory Exchanges and Related Parameters on T₁₄/T₁₅

On T₁₄

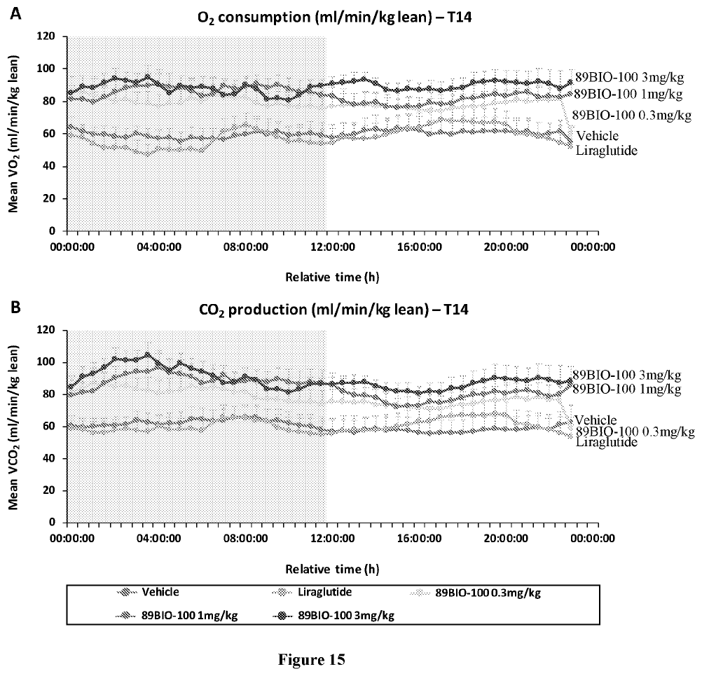

Regarding the analyses of respiratory exchanges on T₁₄ per nycthemeral and as shown in FIGS. 15A/17A, no significant difference in VO₂ was observed between the Liraglutide and the Vehicle groups. However, the VO₂ of mice treated with 89BIO-100 (whatever the dose considered) was increased during the NP and the WD, when compared to the vehicle group. In addition, the VO₂ of mice treated at the dose of 3 mg was also increased during the DP, when compared to the vehicle group.

Moreover, mice treated with 89BIO-100 (whatever the dose considered) showed an increase in VO₂ during the NP and the WD, when compared to the Liraglutide group.

The VO₂ of mice treated at the dose of 3 mg was also increased during the DP, when compared to the Liraglutide group.

Finally, no significant difference in VO₂ was observed between the three 89BIO-100 treated groups.

Regarding the VCO₂ and as shown in FIGS. 15B/17B, no significant difference in VCO₂ was observed between the Liraglutide and the vehicle groups. The VCO₂ of mice treated with 89BIO-100 (whatever the dose considered) was increased during the NP, when compared to the vehicle group. In addition, the VCO₂ of mice treated at the dose of 1 and 3 mg were also increased during the WD, when compared to the vehicle group.

Moreover, mice treated with 89BIO-100 (whatever the dose considered) showed an increase in VCO₂ during the NP, when compared to the Liraglutide group. In addition, the VCO₂ of mice treated at the dose of 1 and 3 mg were also increased during the WD, when compared to the Liraglutide group.

Finally, no significant difference in VCO₂ was observed between the three 89BIO-100 treated groups.

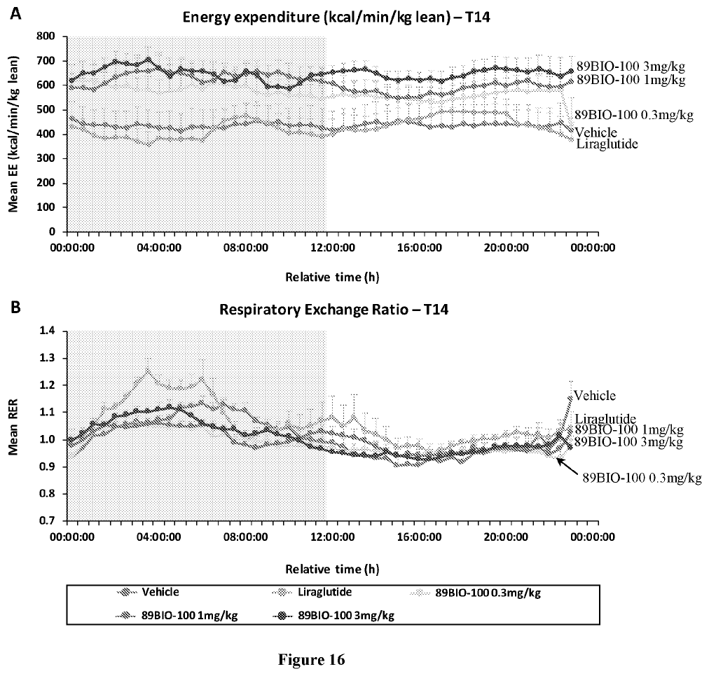

Regarding the EE and as shown in FIGS. 16A/17C, no significant difference in EE was observed between the Liraglutide and the vehicle groups. However, the EE of mice treated with 89BIO-100 (whatever the dose considered) was increased during the NP and the WD, when compared to the vehicle group. In addition, the EE of mice treated at the dose of 3 mg was also increased during the DP, when compared to the Vehicle group.

Moreover, mice treated with 89BIO-100 (whatever the dose considered) showed an increase in EE during the NP and the WD, when compared to the Liraglutide group. In addition, the EE of mice treated at the dose of 3 mg was also increased during the DP, when compared to the Liraglutide group.

Finally, no significant difference in VO₂ was observed between the three 89BIO-100 treated groups.

Regarding the RER and as shown in FIGS. 16B/17D, the RER of Liraglutide treated mice was increased during the WD, when compared to the Vehicle group. No significant difference in RER was observed between mice treated with 89BIO-100 (whatever the dose considered) and mice treated with the vehicle.

Moreover, mice treated with 89BIO-100 (whatever the dose considered) showed a decrease in RER during the NP and the WD, when compared to the Liraglutide group.

Finally, no significant difference in EE was observed between the three 89BIO-100 treated groups.

On T₁₅

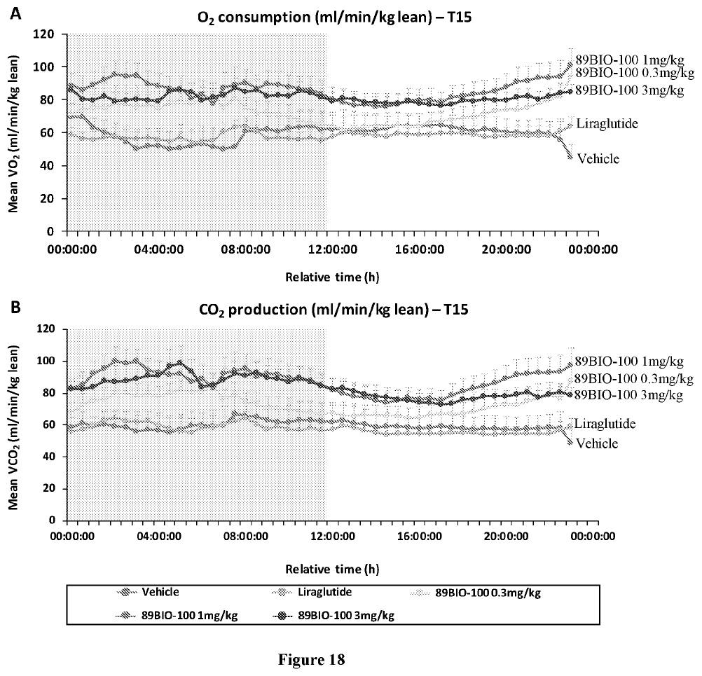

Regarding the analyses of respiratory exchanges on T₁₅ per nycthemeral and as shown in FIGS. 18A/20A, no significant difference in VO₂ was observed between the Liraglutide and the vehicle groups. However, the VO₂ of mice treated with 89BIO-100 at the dose of 1 and 3 mg/kg was increased during the NP and the WD, when compared to the vehicle group. In addition, the VO₂ of mice treated at the dose of 1 mg was also increased during the DP, when compared to the vehicle group.

Moreover, mice treated with 89BIO-100 at the dose of 1 and 3 mg/kg showed an increase in VO₂, when compared to the Liraglutide group and whatever the phase considered.

Finally, no significant difference in VO₂ was observed between the three 89BIO-100 treated groups.

Regarding the VCO₂ and as shown in FIGS. 18B/20B, no significant difference in VCO₂ was observed between the Liraglutide and the vehicle groups. However, the VCO₂ of mice treated with 89BIO-100 at the dose of 1 and 3 mg/kg was increased during the NP and the WD, when compared to the vehicle group. In addition, the VCO₂ of mice treated at the dose of 1 mg was also increased during the DP, when compared to the Vehicle group.

Moreover, mice treated with 89BIO-100 at the dose of 1 and 3 mg/kg showed an increase in VCO₂, when compared to the Liraglutide group and whatever the phase considered.

Finally, no significant difference in VCO₂ was observed between the three 89BIO-100 treated groups.

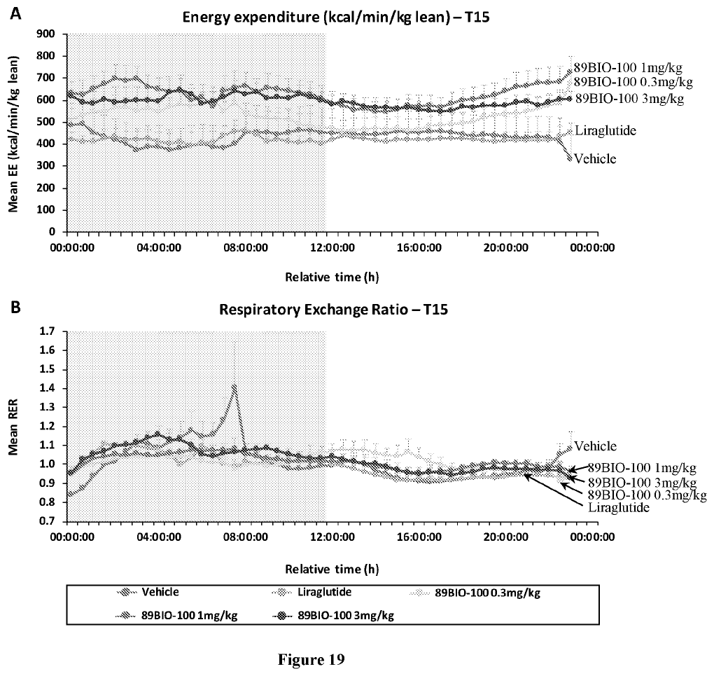

Regarding the EE and as shown in FIGS. 19A/20C, no significant difference in EE was observed between the Liraglutide and the vehicle groups. However, the EE of mice treated with 89BIO-100 at the dose of 1 and 3 mg/kg was increased during the NP and the WD, when compared to the vehicle group. In addition, the EE of mice treated at the dose of 1 mg was also increased during the DP, when compared to the Vehicle group.

Moreover, mice treated with 89BIO-100 at the dose of 1 and 3 mg/kg showed an increase in EE, when compared to the Liraglutide group and whatever the phase considered.

Finally, no significant difference in EE was observed between the three 89BIO-100 treated groups.

Regarding the RER and as shown in FIGS. 19B/20D, no significant difference in RER was observed between the experimental groups.

Impact of 89BIO-100 on respiratory exchanges and related parameters on T₂₆/T₂₇

On T₂₆

Considering the analyses of respiratory exchanges on T₂₆ per nycthemeral, no significant difference in VO₂, VCO₂, EE and RER was observed between the experimental groups.

On T₂₇

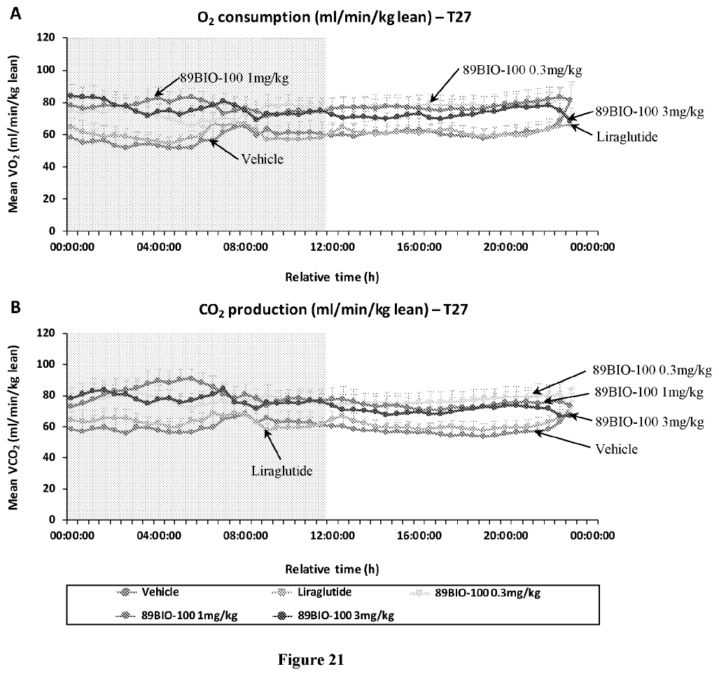

Considering the analyses of respiratory exchanges on T₂₇ per nycthemeral and as shown in FIGS. 21A/23A, no significant difference in VO₂ was observed between the Liraglutide and the vehicle groups. However, the VO₂ of mice treated with 89BIO-100 (whatever the dose considered) was increased during the NP, when compared to the vehicle group. In addition, the VO₂ of mice treated at the dose of 0.3 and 1 mg was also increased during the WD, when compared to the Vehicle group.

Moreover, mice treated with 89BIO-100 at the dose of 1 and 3 mg/kg showed an increase in VO₂ during the NP, when compared to the Liraglutide group. In addition, mice treated with 89BIO-100 at the dose of 0.3 and 1 mg/kg showed an increase in VO₂ during the WD, when compared to the Liraglutide group.

Finally, no significant difference in VO₂ was observed between the three 89BIO-100 treated groups.

Regarding the VCO₂ and as shown in FIGS. 21B/23B, no significant difference in VCO₂ was observed between the Liraglutide and the vehicle groups. However, the VCO₂ of mice treated with 89BIO-100 at the dose of 0.3 and 1 mg/kg was increased during the NP and the WD, when compared to the vehicle group.

Moreover, mice treated with 89BIO-100 at the dose of 1 showed an increase in VCO₂ during the NP and the WD, when compared to the Liraglutide group.

Finally, no significant difference in VCO₂ was observed between the three 89BIO-100 treated groups.

Regarding the EE and as shown in FIGS. 19A/20C, no significant difference in EE was observed between the Liraglutide and the vehicle groups. However, the EE of mice treated with 89BIO-100 (whatever the dose considered) was increased during the NP, when compared to the vehicle group. In addition, the EE of mice treated at the dose of 0.3 and 1 mg was also increased during the WD, when compared to the Vehicle group.

Moreover, mice treated with 89BIO-100 at the dose of 1 showed an increase in EE during the NP, when compared to the Liraglutide group. In addition, the EE of mice treated at the dose of 0.3 and 1 mg was also increased during the WD, when compared to the Liraglutide group.

Finally, no significant difference in EE was observed between the three 89BIO-100 treated groups.

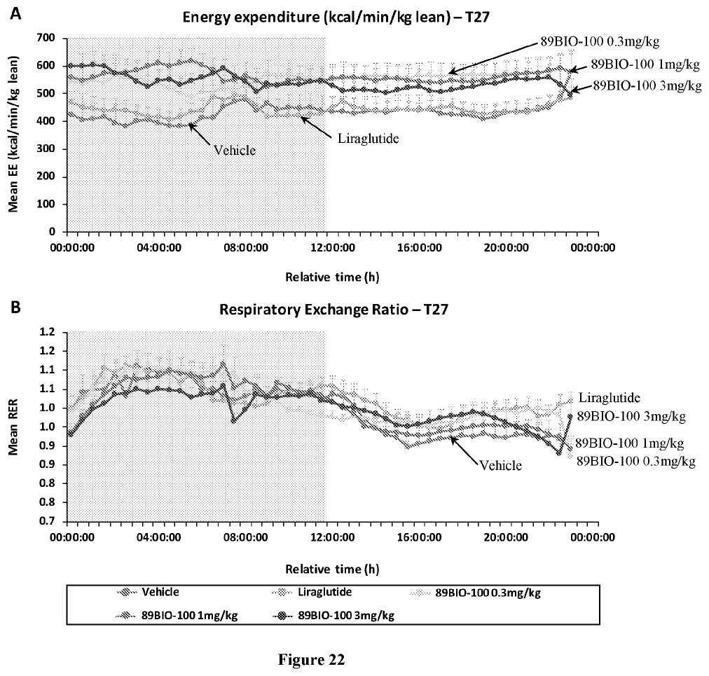

Regarding the RER and as shown in FIGS. 22B/23D, no significant difference in RER was observed between the experimental groups.

### Impact of 89BIO-100 on Total Spontaneous Activity and Rearing

During the three (3) METABOpack sessions (T₁/T₂, T₁₄/T₁₅ and T₂₆/T₂₇), the total spontaneous activity and the rearing of mice were also recorded. Analyses during the diurnal phase (DP), the nocturnal phase (NP) and the whole day (WD) were performed and the obtained results are presented below.

Impact of 89BIO-100 on Total Spontaneous Activity and Rearing on T₁/T₂

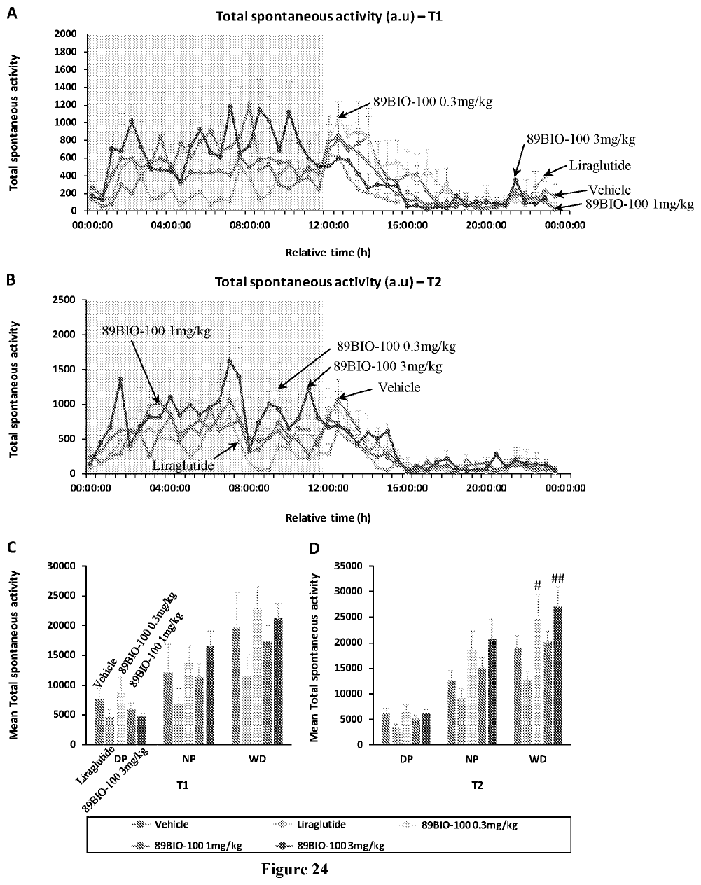

As shown in FIGS. 24A and 24C, there was no statistical difference in total spontaneous activity between the experimental groups, whatever the phase considered on T₁.

As shown in FIGS. 24B and 24D, during the DP and NP of T₂, there was no significant difference between the experimental group.

However, during the WD of T₂, mice treated with 89BIO-100 at the dose of 0.3 and 3 mg/kg exhibited a higher total spontaneous activity than those treated with Liraglutide.

There was no statistical difference in total number of rearing events between the experimental groups, whatever the phase considered on T₁ and T₂.

Impact of 89BIO-100 on Total Spontaneous Activity and Rearing on T₁₄/T₁₅

There was no statistical difference in total spontaneous activity between the experimental groups, whatever the phase considered on T₁₄ and T₁₅.

There was no statistical difference in total number of rearing events between the experimental groups, whatever the phase considered on T₁₄ and T₁₅.

Impact of 89BIO-100 on Total Spontaneous Activity and Rearing on T₂₆/T₂₇

There was no statistical difference in total spontaneous activity between the experimental groups, whatever the phase considered on T₂₆ and T₂₇.

There was no statistical difference in total number of rearing events between the experimental groups, whatever the phase considered on T₂₆ and T₂₇.

### Impact of 89BIO-100 on Body Composition

Body composition were measured three times during the entire study: one (1) day before treatment start (H₇), on T₁₃ and T₂₅.

As shown in FIG. 25A, on H7, just before the treatment, there was no significant difference in fat mass (g) between the experimental groups.

However, on T₁₃, the fat mass (g) of mice treated with Liraglutide was significantly decreased, when compared to the vehicle group. In addition, mice treated with 89BIO-100 at both doses of 1 and 3 mg/kg showed a reduction in fat mass (g), when compared to the vehicle group. No significant difference in fat mass (g) was observed between the three doses of 89BIO-100 and the Liraglutide group.

Moreover, the fat mass (g) of mice treated with 89BIO-100 at the dose of 3 mg/kg was significantly decreased, when compared to mice treated with the dose of 0.3 mg/kg.

Finally, on T₂₅, the fat mass (g) of mice treated with Liraglutide was significantly decreased, when compared to the vehicle group. In addition, mice treated with 89BIO-100 at both doses of 1 and 3 mg/kg showed a reduction in fat mass (g), when compared to the vehicle group. No significant difference in fat mass (g) as observed between the three doses of 89BIO-100 and the Liraglutide group.

The delta body composition between the final (T₂₅) and the initial (H₇) measurement was also calculated. As shown in FIG. 26A, the delta fat mass (g) of mice treated with 89BIO-100 at both doses of 1 and 3 mg/kg was lower, than that of mice treated with vehicle and Liraglutide, respectively.

As shown in FIG. 25B, on H₇, just before the treatment, there was no significant difference in lean mass (g) between the experimental groups.

On T₁₃, there was no significant difference in lean mass (g) between the Liraglutide and vehicle groups. However, the lean mass (g) of mice treated with 89BIO-100 at the dose of 3 mg/kg was significantly decreased, when compared to the vehicle group.

Moreover, mice treated with 89BIO-100 at the dose of 3 mg/kg showed a reduction in lean mass (g), when compared to both doses of 0.3 and 1 mg/kg. No significant difference in lean mass (g) was observed between the three doses of 89BIO-100 and the Liraglutide group.

Moreover, the fat mass (g) of mice treated with 89BIO-100 at the dose of 3 mg/kg was significantly decreased, when compared to mice treated with the dose of 0.3 mg/kg.

Finally, on T₂₅, there was no significant difference in lean mass (g) between the experimental groups.

The delta body composition between the final (T₂₅) and the initial (H₇) measurement was also calculated. As shown in FIG. 26B, no significant difference in the delta lean mass (g) among the experimental groups.

As shown in FIG. 25C, at H₇, just before the treatment, there was no significant difference in fluid mass (g) between the experimental groups.

However, at T₁₃, the fluid mass (g) of mice treated with Liraglutide was decreased, when compared to the Vehicle group. In addition, the fluid mass (g) of mice treated with 89BIO-100 at the dose of 3 mg/kg was significantly decreased, when compared to the Vehicle group.

No significant difference in fluid mass (g) between the three doses of 89BIO-100 and the Liraglutide group.

Moreover, the fluid mass (g) of mice treated with 89BIO-100 at the dose of 3 mg/kg was significantly decreased, when compared to mice treated with the dose of 0.3 mg/kg.

Finally, on T₂₅, there was no significant difference in fluid mass (g) between the experimental groups.

The delta body composition between the final (T₂₅) and the initial (H₇) measurement was also calculated. As shown in FIG. 26C, no significant difference in the delta fluid mass was observed among the experimental groups.

### Impact of 89BIO-100 on Semi-Fasted Blood Glucose

The blood glucose levels of the different groups were measured at different times: prior the treatment (H₇) and during the treatment period (T₃, T₁₆ and T₂₅) after four (4) hours of fasting.

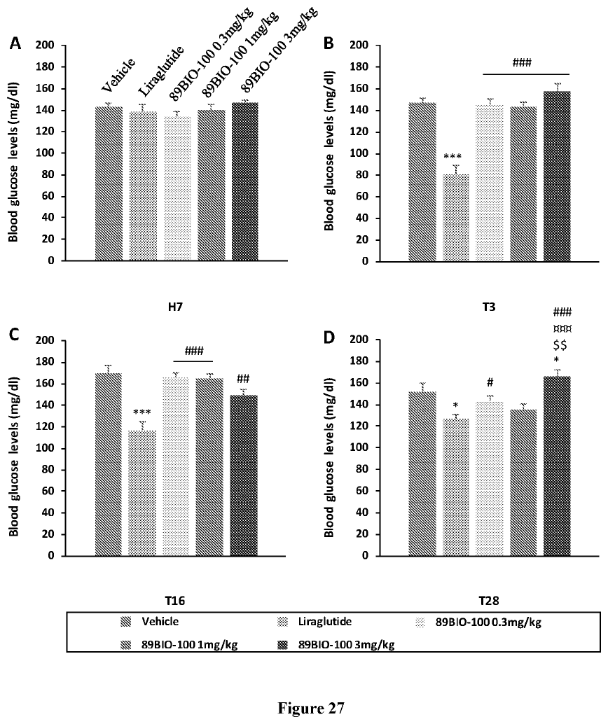

As shown in FIG. 27A, the baseline of blood glucose levels in the different groups was similar prior to any treatment (H₇).

As shown in FIGS. 27A-27D, mice treated with Liraglutide showed a marked decrease in blood glucose levels on T₃, when compared to the vehicle group. In addition, the decrease in blood glucose levels observed in mice treated with Liraglutide was maintained on T₁₆ and T₂₈, when compared to the vehicle group. The treatment with 89BIO-100 (whatever the dose considered) did not alter the blood glucose levels on T₃ and T₁₆, when compared to the vehicle group. However, the dose of 3 mg/kg induced a significant increase in blood glucose levels on T₂₈, when compared to the vehicle group. Moreover, mice treated with 89BIO-100 (whatever the dose considered) showed higher blood glucose levels than those treated with Liraglutide, on T₃ and T₁₆. On T₂₉, only the dose 0.3 and 3 mg/kg increased the blood glucose levels of mice when compared to the Liraglutide group.

Finally, there was no significant difference in blood glucose levels between 89BIO-treated groups themselves on T₃ and T₁₆, except on T₂₈, where mice treated with the dose of 3 mg/kg exhibited higher blood glucose levels than those treated with 0.3 and 1 mg/kg.

### Impact of 89BIO-100 on Organs Weights

On T₂₈, after four (4) hours of fasting, a terminal sampling of blood and organ/tissue were performed on mice at ambient temperature. The following organs/tissues were collected and weighed: liver, gastrocnemius muscle (left side), heart, interscapular brown adipose tissue (iBAT), epididymal white adipose tissue (eWAT; both side) and subcutaneous white adipose tissue (sWAT; both side).

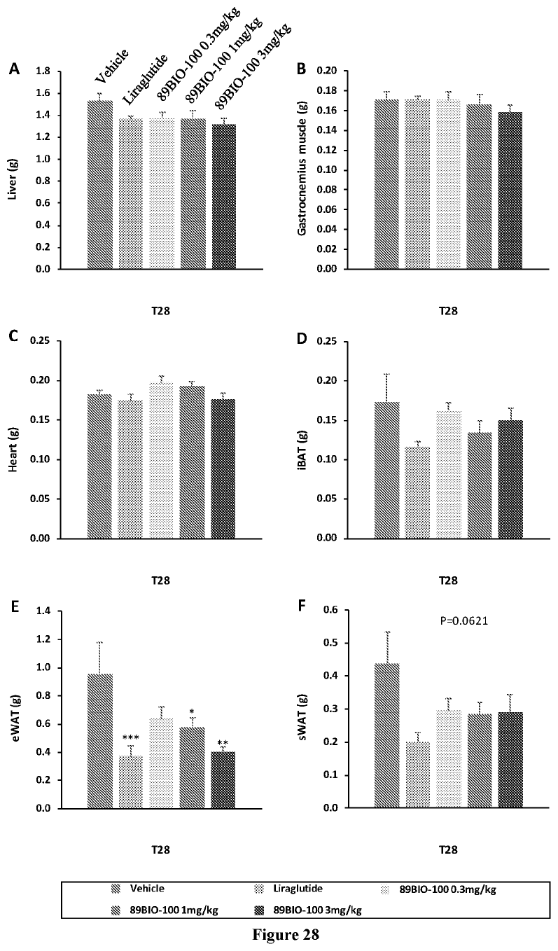

As shown in FIGS. 28A-28F, there was no significant difference in liver, gastrocnemius muscle, heart, iBAT and sWAT weights among the groups. However, the weight of eWAT in mice treated with Liraglutide was decreased, when compared to the vehicle group. Moreover, the treatment with 89BIO-100 at both doses of 1 and 3 mg/kg induced a significant decrease in eWAT weight, when compared to the vehicle group. However, there was no significant difference in eWAT weight among the three 89BIO-100 treated groups.

### Summary

The primary objective of the present project was to investigate the impact of 89BIO-100 on energy expenditure and body weight reduction in CD-1 mice at ambient temperature. To achieve this aim, 6 week-old CD-1 mice were housed at ambient temperature (22.0±1.0° C.) and treated either with Vehicle (3 times/week; sc), Liraglutide at the dose of 0.2 mg/kg (BID; sc; a dose known to be effective in reducing body weight, food intake and increasing insulin sensitivity in obese and diabetic rodent models) or 89BIO-100 at 3 doses (3 times/week; sc). The lowest dose of 89BIO-100 was 0.3 mg/kg, a dose considered to be effective in Diet-induced NASH model in mice. The intermediate dose was 1 mg/kg, a dose used in the 28-day GLP general toxicology study in CD-1 mice and finally, the highest dose chosen for the study was 3 mg/kg.

The body weight and the food intake were daily measured throughout the treatment period. In order to investigate the impact of 89BIO-100 on energy expenditure, the respiratory exchanges (VO₂ and VCO₂) on mice were recorded 3 times (three (3) sessions of METABOpack™): one session at the start of treatment (T₁/T₂), another one in the middle of the treatment period (T₁₄/T₁₅), and the last one at the end of the treatment period (T₂₆/T₂₇). The platform allowed us to also investigate the impact of 89BIO-100 on food intake, meal pattern, water intake and activity in CD-1 mice thanks to the high-resolution recordings of food intake, water intake, total spontaneous activity and rearing. Moreover, a body composition (fat, lean and fluid masses) was performed 3 times during the study. The semi-fasted blood glucose levels were measured 4 times throughout the study and finally, at the end of the study following by a terminal sampling (blood and organ/tissues).

Results showed that mice treated with 89BIO-100 at both 1 and 3 mg/kg exhibited a lower body weight in dose-dependent manner throughout the treatment period. Despite a high heterogeneity within the experimental groups, it appeared that the food intake of mice treated with 89BIO-100 was increased (significantly increased on T₁, T₂ and T₁₅). The increased food intake was associated with an increase in mean meal number without any modulation of mean meal size or satiety ratio. In summarize, even though the CD-1 mice treated with 89BIO-100 at ambient temperature were hyperphagic, they lost weight when compared to the Vehicle group.

Interestingly, results obtained during the different METABOpack sessions brought some explanations on the reduction of body weight. Indeed, during the second (T₁₄/T₁₅) and the third session (T₂₇) of METABOpack, mice treated with 89BIO-100 exhibited an increase in energy expenditure, when compared to the Vehicle group. Thus, the increase in energy expenditure observed in 89BIO-100 treated mice could explain the reduction of body weight of these mice. As a reminder, the energy expenditure is composed of three (3) parameters: basal metabolism, total spontaneous activity and thermogenesis. The increase in energy expenditure could be explained by an increase in one or more parameters. The current study did not give us information on the basal metabolism in these mice. Moreover, results did not show any significant difference in total spontaneous activity among experimental groups, which mean that the increase in energy expenditure observed in 89BIO-100 treated mice could not be explained by an increase in total spontaneous activity. The third parameter, thermogenesis, could explain the increase of energy expenditure in 89BIO-100 mice. To investigate further, it would be interesting to analyze the expression of some thermogenic markers such as UCP-1 (Uncoupling Protein 1), PGC1alpha (Peroxisome proliferator-activated receptor-gamma coactivator 1 alpha), CIDEA (cell death-inducing DNA fragmentation factor alpha-like effector A) in the interscapular brown adipose tissue (iBAT), the major site of thermogenesis, but also in subcutaneous white adipose tissue (sWAT), where the “browning” could occur (UCP-1, PGC1alpha, CIDEA and TBX-1: T-box protein1).

Furthermore, the reduction of body weight observed in 89BIO-100 treated mice was associated with a marked decrease in fat content (g). Indeed, the epididymal white adipose tissue (eWAT) weight was strongly reduced in 89BIO-100 treated mice. It appeared that this tissue could be a key target for 89BIO-100 action. To investigate further, it would be interesting to find out if some genes involved in lipolysis such as HSL (Hormone sensitive lipase), ATGL (adipose triglyceride lipase) and MGL (Monoacylglycerollipase) or in lipogenesis such as FAS (fatty acid synthase) could play a key role in enhancing lipolysis and/or decreasing lipogenesis in eWAT and sWAT.

Finally, results showed any difference in semi-fasted blood glucose between 89BIO-100-treated mice and vehicle-treated mice. In contrast to Liraglutide, 89BIO-100 did not have a hypoglycemic action on wild-type CD-1 mice.

Conclusion: Naive CD-1 mice treated with BIO89-100 had a dose-dependent reduction in BW and increased FC. These results indicate that BIO89-100-mediated body weight loss is due, at least in part, to an increase in energy expenditure, resulting in a marked decrease in fat mass, including the eWAT weight, without affecting lean masses and body fluid.

All publications mentioned herein are hereby incorporated by reference in their entireties. While the foregoing disclosure has been described in some detail for purposes of clarity and understanding, it will be appreciated by one skilled in the art from a reading of the disclosure that various changes in form and detail can be made without departing from the true scope of the disclosure in the appended claims.

Specific examples of methods have been described herein for purposes of illustration. These are only examples. The technology provided herein can be applied to systems other than the example systems described above. Many alterations, modifications, additions, omissions, and permutations are possible within the practice of this disclosure. This disclosure includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

The embodiments of the disclosure described above are intended to be exemplary only. Those skilled in this art will understand that various modifications of detail may be made to these embodiments, all of which come within the scope of the invention.

1. A method, comprising:
administering once a week or once every two weeks to a subject in need thereof a pharmaceutical composition comprising from 0.08 mg/kg to 1 mg/kg of a mutant Fibroblast Growth Factor-21 (FGF-21) peptide conjugate and a pharmaceutically acceptable carrier,
wherein the subject is in need of reduction of total body weight, reduction of body fat content, reduction of body mass index (BMI), or combinations thereof,
wherein the mutant FGF-21 peptide conjugate comprises:
i) a mutant FGF-21 peptide comprising an amino acid sequence of SEQ ID NO: 2,
ii) a glycosyl moiety, and
iii) a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG,
wherein administration of the pharmaceutical composition results in at least one of: reduction of total body weight, reduction of body fat content, reduction of BMI of the subject or combination thereof.

2. (canceled)

3. The method of claim 1, wherein administration of the pharmaceutical composition increases of thermogenesis, decreases in fat mass without affecting lean masses, decreases in fat mass without affecting body fluid or combinations thereof.

4. The method of claim 1, wherein the subject is a human subject.

5. The method of claim 1, wherein the subject is not afflicted with diabetes, NASH, and/or metabolic syndrome.

6. The method of claim 1, wherein the subject has a BMI ranging from 25 to less than 30.

7. The method of claim 1, wherein the subject has a BMI of less than 25.

8. The method of claim 1, wherein the subject has an HbA1C level within normal range of from 4% to 5.6%.

9. The method of claim 1, wherein the subject has a BMI of 30 or greater, and does not have diabetes, NASH, or metabolic syndrome.

10. The method of claim 1, wherein the mutant FGF-21 peptide conjugate exhibit a half life of about 80 hours or greater.

11. The method of claim 1, wherein the pharmaceutical composition is administered in combination with a weight loss therapeutic agent.

12. The method of claim 1, wherein the pharmaceutical composition is administered sub-subcutaneously.

13. The method of claim 1, wherein the glycosyl moiety comprises at least one of an N-acetylgalactosamine (GalNAc) residue, a galactose (Gal) residue, a sialic acid (Sia) residue, a 5-amine analogue of a Sia residue, a mannose (Man) residue, mannosamine, a glucose (Glc) residue, an N-acetylglucosamine (GlcNAc) residue, a fucose residue, a xylose residue, or a combination thereof.

14. (canceled)

15. (canceled)

16. The method of claim 13, wherein the at least one Sia residue is N-acetyl-neuraminic acid (2-keto-5-acetamido-3,5-dideoxy-D-glycero-D-galactononulopyranos-1-onic acid (Neu5Ac), N-glycolylneuraminic acid (Neu5Gc), 2-keto-3-deoxy-nonulosonic acid (KDN), or a 9-substituted sialic acid.

17. The method of claim 16, wherein the 9-substituted sialic acid is 9-O-lactyl-Neu5Ac, 9-O-acetyl-Neu5Ac, 9-deoxy-9-fluoro-Neu5Ac, or 9-azido-9-deoxy-Neu5Ac.

18. The method of claim 1, wherein the glycosyl moiety comprises the structure -GalNAc-Sia-.

19. The method of claim 1, wherein the 20 kDa PEG moiety is attached to the glycosyl moiety by a covalent bond to a linker, wherein the linker comprises at least one amino acid residue.

20. The method of claim 19, wherein the at least one amino acid residue is a glycine (Gly).

21. The method of claim 1, wherein the mutant FGF-21 comprises the structure -GalNAc-Sia-Gly-PEG (20 kDa).

22. The method of claim 1, wherein the mutant FGF-21 comprises the structure: 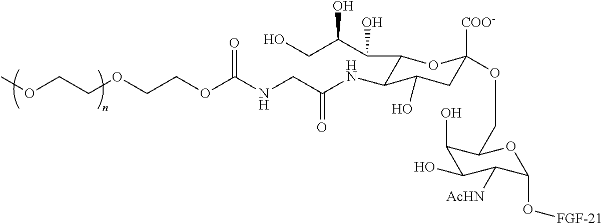
wherein n is an integer selected from 450 to 460.

23. The method of claim 1, wherein the 20 kDa PEG is a linear or branched PEG.

24. The method of claim 1, wherein the 20 kDa PEG is a 20 kDa methoxy-PEG.
